(19) United States
(12) Patent Application Publication
(10) Pub. No.: US 2007/0072813 A1
(43) Pub. Date: Mar. 29, 2007

(54) Methods for preventing and treating metabolic disorders and new pyrazole-O-glycoside derivatives

(76) Inventors: Frank Himmelsbach, Mittelbiberach (DE); Roland Maier, Biberach (DE); Peter Eickelmann, Mittelbiberach (DE); Leo Thomas, Biberach (DE); Edward Leon Barsoumian, Toyonaka (JP); Klaus Dugi, Dresden (DE); Sabine Pinnetti, Biberach (DE); Regine Ritter, Wiesbaden (DE); Ruediger Streicher, Biberach (DE); Koichi Fujita, Yokohama (JP); Toshihiro Hatanaka, Saiwai-ku (JP); Nozomu Ishida, Yokohama (JP); Katsumi Maezono, Kamakura (JP); Koji Ohsumi, Saiwai-ku (JP)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/495,332

(22) Filed: Jul. 28, 2006

(30) Foreign Application Priority Data
Jul. 28, 2005 (EP) 05 016 390

(51) Int. Cl. A61K 31/7052; C07H 17/02

(52) U.S. Cl. 514/23; 536/17.4

(57) ABSTRACT

The invention relates to methods for preventing or treating metabolic disorders, for improving glycemic control, for preventing progression from impaired glucose tolerance, insulin resistance and/or from metabolic syndrome to type 2 diabetes mellitus, for preventing or treating of complications of diabetes mellitus, for reducing the weight, for preventing or treating the degeneration of pancreatic beta cells, for treating hyperinsulinemia and insulin resistance and diabetes type 1, in patients in need thereof by administering a pharmaceutical composition comprising a pyrazole-O-glycoside as defined in claim 1, or a prodrug thereof, or a pharmaceutically acceptable salt thereof.

This application claims priority to EP 05 016 390.6, filed Jul. 28, 2005, the contents of which are incorporated herein.

### TECHNICAL FIELD OF THE INVENTION

The invention relates to methods
for preventing, slowing progression of, delaying, or treating a metabolic disorder;
for improving glycemic control and/or for reducing of fasting plasma glucose, of postprandial plasma glucose and/or of glycosylated hemoglobin HbA1c;
for preventing, slowing, delaying or reversing progression from impaired glucose tolerance, insulin resistance and/or from metabolic syndrome to type 2 diabetes mellitus;
for preventing, slowing progression of, delaying or treating of a condition or disorder selected from the group consisting of complications of diabetes mellitus;
for reducing the weight or preventing an increase of the weight or facilitating a reduction of the weight;
for preventing or treating the degeneration of pancreatic beta cells and/or for improving and/or restoring the functionality of pancreatic beta cells and/or restoring the functionality of pancreatic insulin secretion;
maintaining and/or improving the insulin sensitivity and/or for treating or preventing hyperinsulinemia and/or insulin resistance,
   in patients in need thereof by administering a pharmaceutical composition comprising a pyrazole-O-glucoside derivative selected from the group of compounds (1) to (29) as defined hereinafter, or a prodrug thereof, or a pharmaceutically acceptable salt thereof. In addition the present invention relates to the use of a pyrazole-O-glucoside derivative according to this invention for preparing a pharmaceutical composition and to such medicaments and pharmaceutical compositions.

Furthermore the present invention relates to new pyrazole-O-glucoside derivatives as defined hereinafter, or prodrugs thereof, or pharmaceutically acceptable salts thereof.

The present invention also relates to pharmaceutical compositions comprising at least one of the pyrazole-O-glucoside derivatives as defined hereinafter, or prodrugs thereof, or pharmaceutically acceptable salts thereof.

### BACKGROUND OF THE INVENTION

The European Patent application EP 1 338 603 A1 describes novel pyrazole-O-glycoside derivatives. The pyrazole-O-glycoside derivatives are proposed as inducers of urinary sugar excretion and as medicaments in the treatment of diabetes.

Renal filtration and reuptake of glucose contributes, among other mechanisms, to the steady state plasma glucose concentration and can therefore serve as an antidiabetic target. Reuptake of filtered glucose across epithelial cells of the kidney proceeds via sodium-dependent glucose cotransporters (SGLTs) located in the brush-border membranes in the proximal tubuli along the sodium gradient ⁽¹⁾. There are at least 3 SGLT isoforms that differ in their expression pattern as well as in their physico-chemical properties ⁽²⁾. SGLT2 is exclusively expressed in the kidney⁽³⁾, whereas SGLT1 is expressed additionally in other tissues like intestine, colon, skeletal and cardiac muscle ^{(4;5)}. SGLT3 has been found to be a glucose sensor in interstitial cells of the intestine without any transport function ⁽⁶⁾. Potentially, other related, but not yet characterized genes, may contribute further to renal glucose reuptake ^{(7,8,9)}. Under normoglycemia, glucose is completely reabsorbed by SGLTs in the kidney, whereas the reuptake capacity of the kidney is saturated at glucose concentrations higher than 10 mM, resulting in glucosuria (“diabetes mellitus”). This threshold concentration can be decreased by SGLT2-inhibition. It has been shown in experiments with the SGLT inhibitor phlorizin that SGLT-inhibition will partially inhibit the reuptake of glucose from the glomerular filtrate into the blood leading to a decrease in blood glucose concentrations and to glucosuria ^{(10;11)}.
(1) Wright, E. M. (2001) Am. J. Renal Physiol. 280, F10-F18;
(2) Wright, E. M. et al. (2004) Pflugers Arch. 447(5):510-8;
(3) You, G. et al. (1995) J. Biol. Chem. 270 (49) 29365-29371;
(4) Pajor A M, Wright E M (1992) J Biol. Chem. 267(6):3557-3560;
(5) Zhou, L. et al. (2003) J. Cell. Biochem. 90:339-346;
(6) Diez-Sampedro, A. et al. (2003) Proc. Natl. Acad. Sci. USA 100(20), 11753-11758;
(7) Tabatabai, N. M. (2003) Kidney Int. 64,1320-1330;
(8) Curtis, R. A. J. (2003) US Patent Appl. 2003/0054453;
(9) Bruss, M. and Bonisch, H. (2001) Cloning and functional characterization of a new human sugar transporter in kidney (Genbank Acc. No. AJ305237);
(10) Rossetti, L. Et al. (987) J. Clin. Invest. 79, 1510-1515;
(11) Gouvea, W. L. (1989) Kidney Int. 35(4):1041-1048.

Type 2 diabetes is an increasingly prevalent disease that due to a high frequency of complications leads to a significant reduction of life expectancy. Because of diabetes-associated microvascular complications, type 2 diabetes is currently the most frequent cause of adult-onset loss of vision, renal failure, and amputations in the industrialized world. In addition, the presence of type 2 diabetes is associated with a two to five fold increase in cardiovascular disease risk.

After long duration of disease, most patients with type 2 diabetes will eventually fail on oral therapy and become insulin dependent with the necessity for daily injections and multiple daily glucose measurements.

The UKPDS (United Kingdom Prospective Diabetes Study) demonstrated that intensive treatment with metformin, sulfonylureas or insulin resulted in only a limited improvement of glycemic control (difference in HbA1c ˜0.9%). In addition, even in patients within the intensive treatment arm glycemic control deteriorated significantly over time and this was attributed to deterioration of β-cell function. Importantly, intensive treatment was not associated with a significant reduction in macrovascular complications, i.e. cardiovascular events.

Therefore there is an unmet medical need for drugs with a good efficacy with regard to glycemic control, with regard to disease-modifying properties and with regard to reduction of cardiovascular morbidity and mortality while at the same time showing an improved safety profile.

### Aim of the Present Invention

The aim of the present invention is to provide a method for preventing, slowing progression of, delaying or treating a metabolic disorder.

A further aim of the present invention is to provide a method for improving glycemic control in a patient in need thereof.

Another aim of the present invention is to provide a method for preventing, slowing or delaying progression from impaired glucose tolerance, insulin resistance and/or metabolic syndrome to type 2 diabetes mellitus.

Yet another aim of the present invention is to provide a method for preventing, slowing progression of, delaying or treating of a condition or disorder from the group consisting of complications of diabetes mellitus.

A further aim of the present invention is to provide a method for reducing the weight or preventing an increase of the weight in a patient in need thereof.

Further aims of the present invention relate to new uses of pyrazole-O-glucoside derivatives according to this invention, including prodrugs and pharmaceutically acceptable salts thereof.

Another aim of the present invention is to provide new pyrazole-O-glucoside derivatives and new prodrugs of pyrazole-O-glucoside derivatives thereof which have a good to very good inhibitory effect on the sodium-dependent glucose cotransporter SGLT, in particular SGLT2, in vitro and/or in vivo and/or have good to very good pharmacological and/or pharmacokinetic and/or physicochemical properties.

Further aims of the present invention become apparent to the one skilled in the art by description hereinbefore and in the following and by the examples.

### SUMMARY OF THE INVENTION

Within the scope of the present invention it has now surprisingly been found that a pyrazole-O-glucoside derivative selected from the group of compounds (1) to (29), or prodrugs thereof, or pharmaceutically acceptable salts thereof, as defined hereinafter can advantageously be used in preventing, slowing progression of, delaying or treating a metabolic disorder, in particular in improving glycemic control in patients. This opens up new therapeutic possibilities in the treatment and prevention of type 2 diabetes mellitus, overweight, obesity, complications of diabetes mellitus and of neighboring disease states.

Therefore in a first aspect the present invention provides a method for preventing, slowing progression of, delaying or treating a metabolic disorder selected from the group consisting of type 1 diabetes mellitus, type 2 diabetes mellitus, impaired glucose tolerance, hyperglycemia, postprandial hyperglycemia, overweight, obesity, including class I obesity, class II obesity, class III obesity, visceral obesity and abdominal obesity, and metabolic syndrome in a patient in need thereof characterized in that a pharmaceutical composition comprising a pyrazole-O-glucoside derivative selected from the group of compounds (1) to (29) consisting of
(1) 4-(2,3-difluoro-4-methoxy-benzyl)-1-isopropyl-5-methyl-3-β-D-glucopyranos-1-yloxy-1H-pyrazole;
(2) 4-(2,5-difluoro-4-methoxy-benzyl)-1-isopropyl-5-methyl-3-β-D-glucopyranos-1-yloxy-1H-pyrazole;
(3) 4-(2,6-difluoro-4-methoxy-benzyl)-1-isopropyl-5-methyl-3-β-D-glucopyranos-1-yloxy-1H-pyrazole;
(4) 4-(3,5-difluoro-4-methoxy-benzyl)-1-isopropyl-5-methyl-3-β-D-glucopyranos-1-yloxy-1H-pyrazole;
(5) 1-cyclobutyl-4-(3-fluoro-4-methyl-benzyl)-5-methyl-3-β-D-glucopyranos-1-yloxy-1H-pyrazole;
(6) 1-cyclopropylmethyl-4-(3-fluoro-4-methyl-benzyl)-5-methyl-3-β-D-glucopyranos-1-yloxy-1H-pyrazole;
(7) 1-cyclobutyl-4-(2-fluoro-4-methoxy-benzyl)-5-methyl-3-β-D-glucopyranos-1-yloxy-1H-pyrazole;
(8) 4-(3-chloro-4-methoxy-benzyl)-1-isopropyl-5-methyl-3-β-D-glucopyranos-1-yloxy-1H-pyrazole;
(9) 4-(2-chloro-4-methoxy-benzyl)-1-isopropyl-5-methyl-3-β-D-glucopyranos-1-yloxy-1H-pyrazole;
(10) 4-(4-bromo-3-fluoro-benzyl)-1-isopropyl-5-methyl-3-β-D-glucopyranos-1-yloxy-1H-pyrazole;
(11) 4-(2,3-difluoro-4-methyl-benzyl)-1-isopropyl-5-methyl-3-β-D-glucopyranos-1-yloxy-1H-pyrazole;
(12) 4-(2-fluoro-4-methyl-benzyl)-1-isopropyl-5-methyl-3-β-D-glucopyranos-1-yloxy-1H-pyrazole;
(13) 4-(3-fluoro-4-ethoxy-benzyl)-1-isopropyl-5-methyl-3-β-D-glucopyranos-1-yloxy-1H-pyrazole;
(14) 4-(4-ethinyl-benzyl)-1-isopropyl-5-methyl-3-β-D-glucopyranos-1-yloxy-1H-pyrazole;
(15) 4-(3-fluoro-4-isopropoxy-benzyl)-1-isopropyl-5-methyl-3-β-D-glucopyranos-1-yloxy-1H-pyrazole;
(16) 4-(2-fluoro-4-methoxy-benzyl)-1-isopropyl-5-methyl-3-β-D-glucopyranos-1-yloxy-1H-pyrazole;
(17) 4-(2-fluoro-4-methoxy-benzyl)-1-isopropyl-5-trifluoromethyl-3-β-D-glucopyranos-1-yloxy-1H-pyrazole;
(18) 4-(4-bromo-2-fluoro-benzyl)-1-isopropyl-5-methyl-3-β-D-glucopyranos-1-yloxy-1H-pyrazole;
(19) 4-(2-fluoro-4-isopropoxy-benzyl)-1-isopropyl-5-methyl-3-β-D-glucopyranos-1-yloxy-1H-pyrazole;
(20) 4-(2-fluoro-4-ethoxy-benzyl)-1-isopropyl-5-methyl-3-β-D-glucopyranos-1-yloxy-1H-pyrazole;
(21) 4-(4-ethyl-benzyl)-1-isopropyl-5-trifluoromethyl-3-β-D-glucopyranos-1-yloxy-1H-pyrazole;
(22) 4-(4-bromo-benzyl)-1-isopropyl-5-trifluoromethyl-3-β-D-glucopyranos-1-yloxy-1H-pyrazole;
(23) 4-(4-ethyl-benzyl)-1-cyclobutyl-5-trifluoromethyl-3-β-D-glucopyranos-1-yloxy-1H-pyrazole;
(24) 4-(4-ethyl-benzyl)-1-(2-fluoro-1-fluoromethyl-ethyl)-5-trifluoromethyl-3-β-D-glucopyranos-1-yloxy-1H-pyrazole;
(25) 4-(3-fluoro-4-methoxy-benzyl)-1-isopropyl-5-trifluoromethyl-3-β-D-glucopyranos-1-yloxy-1H-pyrazole;
(26) 4-(3-fluoro-4-methyl-benzyl)-1-isopropyl-5-methyl-3-β-D-glucopyranos-1-yloxy-1H-pyrazole;
(27) 4-(2,3-difluoro-4-isopropoxy-benzyl)-1-isopropyl-5-methyl-3-β-D-glucopyranos-1-yloxy-1H-pyrazole;
(28) 4-(3-fluoro-4-methoxy-benzyl)-1-isopropyl-5-methyl-3-β-D-glucopyranos-1-yloxy-1H-pyrazole;
(29) 4-(4-ethyl-benzyl)-1-isopropyl-5-methyl-3-β-D-glucopyranos-1-yloxy-1H-pyrazole;
   or a prodrug thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl, phenyl-(C₁₋₃-alkyl)-carbonyl, phenyloxycarbonyl and phenyl-(C₁₋₃-alkyl)-oxycarbonyl, or a pharmaceutically acceptable salt thereof;
is administered.

According to another aspect of the invention there is provided a method for improving glycemic control and/or for reducing of fasting plasma glucose, of postprandial plasma glucose and/or of glycosylated hemoglobin HbA1c in a patient in need thereof characterized in that a pharmaceutical composition comprising a pyrazole-O-glucoside derivative selected from the group of compounds (1) to (29), or a prodrug thereof, or a pharmaceutically acceptable salt thereof, defined as hereinbefore and hereinafter is administered.

As by the use of a compound according to this invention an improvement of the glycemic control in patients in need thereof is obtainable, also those conditions and/or diseases related to or caused by an increased blood glucose level may be treated.

Therefore in another aspect the invention provides a method for preventing, slowing progression of, delaying or treating of a condition or disorder selected from the group consisting of complications of diabetes mellitus such as cataracts and micro- and macrovascular diseases, such as nephropathy, retinopathy, neuropathy, tissue ischaemia, arteriosclerosis, myocardial infarction, stroke and peripheral arterial occlusive disease, in a patient in need thereof characterized in that a pharmaceutical composition comprising a pyrazole-O-glucoside derivative selected from the group of compounds (1) to (29), or a prodrug thereof, or a pharmaceutically acceptable salt thereof, defined as hereinbefore and hereinafter is administered. The term “tissue ischaemia” particularly comprises diabetic macroangiopathy, diabetic microangiopathy, impaired wound healing and diabetic ulcer.

The compounds according to this invention may also have valuable disease-modifying properties with respect to diseases or conditions related to impaired glucose tolerance, insulin resistance and/or metabolic syndrome.

Therefore in another aspect of the present invention there is provided a method for preventing, slowing, delaying or reversing progression from impaired glucose tolerance, insulin resistance and/or from metabolic syndrome to type 2 diabetes mellitus in a patient in need thereof characterized in that a pharmaceutical composition comprising a pyrazole-O-glucoside derivative selected from the group of compounds (1) to (29), or a prodrug thereof, or a pharmaceutically acceptable salt thereof, defined as hereinbefore and hereinafter is administered.

By the administration of a compound according to this invention excessive blood glucose levels are not converted to insoluble storage forms, like fat, but excreted through the urine of the patient. Therefore no gain in weight or even a reduction of the weight is the result.

Following this another aspect of the present invention provides a method for reducing the weight or preventing an increase of the weight or facilitating a reduction of the weight in a patient in need thereof characterized in that a pharmaceutical composition comprising a pyrazole-O-glucoside derivative selected from the group of compounds (1) to (29), or a prodrug thereof, or a pharmaceutically acceptable salt thereof, defined as hereinbefore and hereinafter is administered.

The pharmacological effect of the compounds according to this invention is independent of insulin. Therefore an improvement of the glycemic control is possible without an additional strain on the pancreatic beta cells. By an administration of a compound according to this invention a beta-cell degeneration and a decline of beta-cell functionality such as for example apoptosis or necrosis of pancreatic beta cells can be delayed or prevented. Furthermore the functionality of pancreatic cells can be improved or restored, and the number and size of pancreatic beta cells increased. It may be shown that the differentiation status and hyperplasia of pancreatic beta-cells disturbed by hyperglycemia can be normalized by treatment with a compound according to this invention.

Therefore another aspect of the present invention provides a method for preventing, slowing, delaying or treating the degeneration of pancreatic beta cells and/or the decline of the functionality of pancreatic beta cells and/or for improving and/or restoring the functionality of pancreatic beta cells and/or restoring the functionality of pancreatic insulin secretion in a patient in need thereof characterized in that a pharmaceutical composition comprising a pyrazole-O-glucoside derivative selected from the group of compounds (1) to (29), or a prodrug thereof, or a pharmaceutically acceptable salt thereof, defined as hereinbefore and hereinafter is administered.

As a result thereof another aspect of the present invention provides a method for maintaining and/or improving the insulin sensitivity and/or for treating or preventing hyperinsulinemia and/or insulin resistance in a patient in need thereof characterized in that a pharmaceutical composition comprising a pyrazole-O-glucoside derivative selected from the group of compounds (1) to (29), or a prodrug thereof, or a pharmaceutically acceptable salt thereof, defined as hereinbefore and hereinafter is administered.

Other aspects of the present invention relate to the use of a pyrazole-O-glucoside derivative selected from the group of compounds (1) to (29), or a prodrug thereof, or a pharmaceutically acceptable salt thereof, defined as hereinbefore and hereinafter in the treatment or prophylaxis of diseases or conditions as described hereinbefore and hereinafter.

Further aspects of the present invention relate to the use of a pyrazole-O-glucoside derivative selected from the group of compounds (1) to (29), or a prodrug thereof, or a pharmaceutically acceptable salt thereof, defined as hereinbefore and hereinafter for the manufacture of a medicament for a therapeutic method as described hereinbefore and hereinafter.

Furthermore another aspect of the present invention relates to a medicament or pharmaceutical composition comprising a therapeutically or prophylactically effective amount of a pyrazole-O-glucoside derivative selected from the group of compounds (1) to (29), or a prodrug thereof, or a pharmaceutically acceptable salt thereof, defined as hereinbefore and hereinafter for the treatment or prophylaxis of diseases or conditions as described hereinbefore and hereinafter.

Another aspect of the present invention relates to novel pyrazole-O-glucoside derivatives selected from the group consisting of:
(1) 4-(2,3-difluoro-4-methoxy-benzyl)-1-isopropyl-5-methyl-3-β-D-glucopyranos-1-yloxy-1H-pyrazole;
(2) 4-(2,5-difluoro-4-methoxy-benzyl)-1-isopropyl-5-methyl-3-β-D-glucopyranos-1-yloxy-1H-pyrazole;
(3) 4-(2,6-difluoro-4-methoxy-benzyl)-1-isopropyl-5-methyl-3-β-D-glucopyranos-1-yloxy-1H-pyrazole;
(4) 4-(3,5-difluoro-4-methoxy-benzyl)-1-isopropyl-5-methyl-3-β-D-glucopyranos-1-yloxy-1H-pyrazole;
(5) 1-cyclobutyl-4-(3-fluoro-4-methyl-benzyl)-5-methyl-3-β-D-glucopyranos-1-yloxy-1H-pyrazole;
(6) 1-cyclopropylmethyl-4-(3-fluoro-4-methyl-benzyl)-5-methyl-3-β-D-glucopyranos-1-yloxy-1H-pyrazole;
(7) 1-cyclobutyl-4-(2-fluoro-4-methoxy-benzyl)-5-methyl-3-β-D-glucopyranos-1-yloxy-1H-pyrazole;
(8) 4-(3-chloro-4-methoxy-benzyl)-1-isopropyl-5-methyl-3-β-D-glucopyranos-1-yloxy-1H-pyrazole;
(9) 4-(2-chloro-4-methoxy-benzyl)-1-isopropyl-5-methyl-3-β-D-glucopyranos-1-yloxy-1H-pyrazole;
(10) 4-(4-bromo-3-fluoro-benzyl)-1-isopropyl-5-methyl-3-β-D-glucopyranos-1-yloxy-1H-pyrazole;
(11) 4-(2,3-difluoro-4-methyl-benzyl)-1-isopropyl-5-methyl-3-β-D-glucopyranos-1-yloxy-1H-pyrazole;
(12) 4-(2-fluoro-4-methyl-benzyl)-1-isopropyl-5-methyl-3-β-D-glucopyranos-1-yloxy-1H-pyrazole;
(13) 4-(3-fluoro-4-ethoxy-benzyl)-1-isopropyl-5-methyl-3-β-D-glucopyranos-1-yloxy-1H-pyrazole;
(14) 4-(4-ethinyl-benzyl)-1-isopropyl-5-methyl-3-β-D-glucopyranos-1-yloxy-1H-pyrazole;
(15) 4-(3-fluoro-4-isopropoxy-benzyl)-1-isopropyl-5-methyl-3-β-D-glucopyranos-1-yloxy-1H-pyrazole;
(17) 4-(2-fluoro-4-methoxy-benzyl)-1-isopropyl-5-trifluoromethyl-3-β-D-glucopyranos-1-yloxy-1H-pyrazole;
(18) 4-(4-bromo-2-fluoro-benzyl)-1-isopropyl-5-methyl-3-β-D-glucopyranos-1-yloxy-1H-pyrazole;
(19) 4-(2-fluoro-4-isopropoxy-benzyl)-1-isopropyl-5-methyl-3-β-D-glucopyranos-1-yloxy-1H-pyrazole;
(20) 4-(2-fluoro-4-ethoxy-benzyl)-1-isopropyl-5-methyl-3-β-D-glucopyranos-1-yloxy-1H-pyrazole;
   or a prodrug thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl, phenyl-(C₁₋₃-alkyl)-carbonyl, phenyloxycarbonyl and phenyl-(C₁₋₃-alkyl)-oxycarbonyl, or a pharmaceutically acceptable salt thereof;

Yet another aspect of the present invention relates to novel prodrugs of pyrazole-O-glucoside derivatives selected from the group consisting of:
(46) 4-(3-Fluoro-4-methyl-benzyl)-1-isopropyl-5-methyl-3-(6-O-ethoxycarbonyl-β-D-glucopyranos-1-yloxy)-1H-pyrazole;
(47) 4-(2-Fluoro-4-methoxy-benzyl)-1-isopropyl-5-methyl-3-(6-O-ethoxycarbonyl-β-D-glucopyranos-1-yloxy)-1H-pyrazole;
(48) 4-(2-Fluoro-4-methoxy-benzyl)-1-isopropyl-5-methyl-3-(6-O-isobutyloxycarbonyl-β-D-glucopyranos-1-yloxy)-1H-pyrazole;
(49) 4-(2-Fluoro-4-methoxy-benzyl)-1-isopropyl-5-methyl-3-(6-O-hex-1-yloxycarbonyl-β-D-glucopyranos-1-yloxy)-1H-pyrazole;
(50) 4-(2-Fluoro-4-methoxy-benzyl)-1-isopropyl-5-methyl-3-(6-O-phenoxycarbonyl-β-D-glucopyranos-1-yloxy)-1H-pyrazole;
(51) 4-(2-Fluoro-4-methoxy-benzyl)-1-isopropyl-5-methyl-3-(6-O-benzyloxycarbonyl-β-D-glucopyranos-1-yloxy)-1H-pyrazole;
(52) 4-(2-Fluoro-4-methoxy-benzyl)-1-isopropyl-5-methyl-3-(6-O-acetyl-β-D-glucopyranos-1-yloxy)-1H-pyrazole;
(53) 4-(2-Fluoro-4-methoxy-benzyl)-1-isopropyl-5-methyl-3-(6-O-propylcarbonyl-β-D-glucopyranos-1-yloxy)-1H-pyrazole;
(54) 4-(2-Fluoro-4-methoxy-benzyl)-1-isopropyl-5-methyl-3-(6-O-isopropylcarbonyl-β-D-glucopyranos-1-yloxy)-1H-pyrazole;
(55) 4-(2-Fluoro-4-methoxy-benzyl)-1-isopropyl-5-methyl-3-(6-O-benzylcarbonyl-β-D-glucopyranos-1-yloxy)-1H-pyrazole;
(56) 4-(4-ethyl-benzyl)-1-isopropyl-5-trifluoromethyl-3-(6-O-ethoxycarbonyl-β-D-glucopyranos-1-yloxy)-1H-pyrazole;
(57) 4-(4-bromo-benzyl)-1-isopropyl-5-trifluoromethyl-3-(6-O-ethoxycarbonyl-β-D-glucopyranos-1-yloxy)-1H-pyrazole;
(58) 4-(4-ethyl-benzyl)-1-cyclobutyl-5-trifluoromethyl-3-(6-O-ethoxycarbonyl-β-D-glucopyranos-1-yloxy)-1H-pyrazole;
(59) 4-(4-ethyl-benzyl)-1-(2-fluoro-1-fluoromethyl-ethyl)-5-trifluoromethyl-3-(6-O-ethoxycarbonyl-β-D-glucopyranos-1-yloxy)-1H-pyrazole;
(60) 4-(3-fluoro-4-methoxy-benzyl)-1-isopropyl-5-trifluoromethyl-3-(6-O-ethoxycarbonyl-β-D-glucopyranos-1-yloxy)-1H-pyrazole;
(61) 4-(4-isopropoxy-benzyl)-1-isopropyl-5-methyl-3-(6-O-ethoxycarbonyl-β-D-glucopyranos-1-yloxy)-1H-pyrazole;
(62) 4-(3-fluoro-4-methoxy-benzyl)-1-isopropyl-5-methyl-3-(6-O-ethoxycarbonyl-β-D-glucopyranos-1-yloxy)-1H-pyrazole;
(63) 4-(4-ethyl-benzyl)-1-isopropyl-5-methyl-3-(6-O-ethoxycarbonyl-β-D-glucopyranos-1-yloxy)-1H-pyrazole;
   or pharmaceutically acceptable salts thereof.

A further aspect of the present invention relates to pharmaceutical compositions comprising at least one pyrazole-O-glucoside derivative according to this invention, or a pharmaceutically acceptable salt thereof.

### DEFINITIONS

The term “body mass index” or “BMI” of a human patient is defined as the weight in kilograms divided by the square of the height in meters, such that BMI has units of kg/m².

The term “overweight” is defined as the condition wherein the individual has a BMI greater than or 25 kg/M² and less than 30 kg/M². The terms “overweight” and “pre-obese” are used interchangeably.

The term “obesity” is defined as the condition wherein the individual has a BMI equal to or greater than 30 kg/M². According to a WHO definition the term obesity may be categorized as follows: the term “class I obesity” is the condition wherein the BMI is equal to or greater than 30 kg/m² but lower than 35 kg/m²; the term “class 11 obesity” is the condition wherein the BMI is equal to or greater than 35 kg/M² but lower than 40 kg/M²; the term “class III obesity” is the condition wherein the BMI is equal to or greater than 40 kg/M².

The term “visceral obesity” is defined as the condition wherein a waist-to-hip ratio of greater than or equal to 1.0 in men and 0.8 in women is measured. It defines the risk for insulin resistance and the development of pre-diabetes.

The term “abdominal obesity” is usually defined as the condition wherein the waist circumference is >40 inches or 102 cm in men, and is >35 inches or 94 cm in women. With regard to a Japanese ethnicity or Japanese patients abdominal obesity may be defined as waist circumference ≧85 cm in men and ≧90 cm in women (see e.g. investigating committee for the diagnosis of metabolic syndrome in Japan).

The term “euglycemia” is defined as the condition in which a subject has a fasting blood glucose concentration within the normal range, greater than 70 mg/dL (3.89 mmol/L) and less than 110 mg/dL (6.11 mmol/L). The word “fasting” has the usual meaning as a medical term.

The term “hyperglycemia” is defined as the condition in which a subject has a fasting blood glucose concentration above the normal range, greater than 110 mg/dL (6.11 mmol/L). The word “fasting” has the usual meaning as a medical term.

The term “postprandial hyperglycemia” is defined as the condition in which a subject has a 2 hour postprandial blood glucose or serum glucose concentration greater than 200 mg/dL (11.11 mmol/L).

The term “impaired glucose tolerance” or “IGT”, is defined as the condition in which a subject has a fasting blood glucose concentration or fasting serum glucose concentration greater than 110 mg/dL and less than 126 mg/dl (7.00 mmol/L), or a 2 hour postprandial blood glucose or serum glucose concentration greater than 140 mg/dl (7.78 mmol/L) and less than 200 mg/dL (11.11 mmol/L). The term impaired glucose tolerance is also intended to apply to the condition of impaired fasting glucose. The abnormal glucose tolerance, i.e. the 2 hour postprandial blood glucose or serum glucose concentration can be measured as the blood sugar level in mg of glucose per dL of plasma 2 hours after taking 75 g of glucose after a fast.

The term “hyperinsulinemia” is defined as the condition in which a subject with insulin resistance, with or without euglycemia, in which the fasting or postprandial serum or plasma insulin concentration is elevated above that of normal, lean individuals without insulin resistance, having a waist-to-hip ration <1.0 (for men) or <0.8 (for women).

The terms “insulin-sensitizing”, “insulin resistance-improving” or “insulin resistance-lowering” are synonymous and used interchangeably.

The term “insulin resistance” is defined as a state in which circulating insulin levels in excess of the normal response to a glucose load are required to maintain the euglycemic state (Ford E S, et al. JAMA. (2002) 287:356-9). A method of determining insulin resistance is the euglycaemic-hyperinsulinaemic clamp test. The ratio of insulin to glucose is determined within the scope of a combined insulin-glucose infusion technique. There is found to be insulin resistance if the glucose absorption is below the 25th percentile of the background population investigated (WHO definition). Rather less laborious than the clamp test are so called minimal models in which, during an intravenous glucose tolerance test, the insulin and glucose concentrations in the blood are measured at fixed time intervals and from these the insulin resistance is calculated. In this method it is not possible to distinguish between hepatic and peripheral insulin resistance.

Furthermore insulin resistance, the response of a patient with insulin resistance to therapy, insulin sensitivity and hyperinsulinemia may be quantified by assessing the “homeostasis model assessment to insulin resistance (HOMA-IR)” score, a reliable indicator of insulin resistance (Katsuki A, et al. Diabetes Care 2001; 24: 362-5). Further reference is made to methods for the determination of the HOMA-index for insulin sensitivity (Matthews et al., Diabetologia 1985, 28: 412-19), of the ratio of intact proinsulin to insulin (Forst et al., Diabetes 2003, 52(Suppl. 1): A459) and to an euglycemic clamp study. In addition, plasma adiponectin levels can be monitored as a potential surrogate of insulin sensitivity. The estimate of insulin resistance by the homeostasis assessment model (HOMA)-IR score is calculated with the formula (Galvin P, et al. Diabet Med 1992; 9:921-8):
HOMA-IR 32 [fasting serum insulin (μU/mL)]×[fasting plasma glucose(mmol/L)/22.5]

As a rule, other parameters are used in everyday clinical practice to assess insulin resistance. Preferably, the patient's triglyceride concentration is used, for example, as increased triglyceride levels correlate significantly with the presence of insulin resistance.

Patients with a predisposition for the development of IGT or type 2 diabetes are those having euglycemia with hyperinsulinemia and are by definition, insulin resistant. A typical patient with insulin resistance is usually overweight or obese. If insulin resistance can be detected this is a particularly strong indication of the presence of prediabetes. Thus, it may be that in order to maintain glucose homoeostasis a person needs 2-3 times as much insulin as another person, without this having any direct pathological significance.

The methods to investigate the function of pancreatic beta-cells are similar to the above methods with regard to insulin sensitivity, hyperinsulinemia or insulin resistance: An improvement of the beta-cell function can be measured for example by determining a HOMA-index for beta-cell function (Matthews et al., Diabetologia 1985, 28: 412-19), the ratio of intact proinsulin to insulin (Forst et al., Diabetes 2003, 52(Suppl. 1): A459), the insulin/C-peptide secretion after an oral glucose tolerance test or a meal tolerance test, or by employing a hyperglycemic clamp study and/or minimal modeling after a frequently sampled intravenous glucose tolerance test (Stumvoll et al., Eur J Clin Invest 2001, 31: 380-81).

The term “pre-diabetes” is the condition wherein an individual is pre-disposed to the development of type 2 diabetes. Pre-diabetes extends the definition of impaired glucose tolerance to include individuals with a fasting blood glucose within the high normal range ≧100 mg/dL (J. B. Meigs, et al. Diabetes 2003; 52:1475-1484) and fasting hyperinsulinemia (elevated plasma insulin concentration). The scientific and medical basis for identifying pre-diabetes as a serious health threat is laid out in a Position Statement entitled “The Prevention or Delay of Type 2 Diabetes” issued jointly by the American Diabetes Association and the National Institute of Diabetes and Digestive and Kidney Diseases (Diabetes Care 2002; 25:742-749).

Individuals likely to have insulin resistance are those who have two or more of the following attributes: 1) overweight or obese, 2) high blood pressure, 3) hyperlipidemia, 4) one or more 1^{st} degree relative with a diagnosis of IGT or type 2 diabetes. Insulin resistance can be confirmed in these individuals by calculating HOMA-IR score. For the purpose of this invention, insulin resistance is defined as the clinical condition in which an individual has a HOMA-IR score >4.0 or a HOMA-IR score above the upper limit of normal as defined for the laboratory performing the glucose and insulin assays.

The term “type 2 diabetes” is defined as the condition in which a subject has a fasting blood glucose or serum glucose concentration greater than 125 mg/dL (6.94 mmol/L). The measurement of blood glucose values is a standard procedure in routine medical analysis. If a glucose tolerance test is carried out, the blood sugar level of a diabetic will be in excess of 200 mg of glucose per dL of plasma 2 hours after 75 g of glucose have been taken on an empty stomach. In a glucose tolerance test 75 g of glucose are administered orally to the patient being tested after 10-12 hours of fasting and the blood sugar level is recorded immediately before taking the glucose and 1 and 2 hours after taking it. In a healthy subject the blood sugar level before taking the glucose will be between 60 and 110 mg per dL of plasma, less than 200 mg per dL 1 hour after taking the glucose and less than 140 mg per dL after 2 hours. If after 2 hours the value is between 140 and 200 mg this is regarded as abnormal glucose tolerance.

The term “late stage type 2 diabetes mellitus” includes patients with a secondary drug failure, indication for insulin therapy and progression to micro- and macrovascular complications e.g. diabetic nephropathy, coronary heart disease (CHD).

The term “HbA1c” refers to the product of a non-enzymatic glycation of the haemoglobin B chain. Its determination is well known to one skilled in the art. In monitoring the treatment of diabetes mellitus the HbA1c value is of exceptional importance. As its production depends essentially on the blood sugar level and the life of the erythrocytes, the HbA1c in the sense of a “blood sugar memory” reflects the average blood sugar levels of the preceding 4-6 weeks. Diabetic patients whose HbA1c value is consistently well adjusted by intensive diabetes treatment (i.e. <6.5% of the total haemoglobin in the sample), are significantly better protected against diabetic microangiopathy. For example metformin on its own achieves an average improvement in the HbA1c value in the diabetic of the order of 1.0-1.5 %. This reduction of the HbA1C value is not sufficient in all diabetics to achieve the desired target range of <6.5% and preferably <6% HbA1c.

The “metabolic syndrome”, also called “syndrome X” (when used in the context of a metabolic disorder), also called the “dysmetabolic syndrome” is a syndrome complex with the cardinal feature being insulin resistance (Laaksonen D E, et al. Am J Epidemiol 2002; 156:1070-7). According to the ATP III/NCEP guidelines (Executive Summary of the Third Report of the National Cholesterol Education Program (NCEP) Expert Panel on Detection, Evaluation, and Treatment of High Blood Cholesterol in Adults (Adult Treatment Panel III) JAMA: Joumal of the American Medical Association (2001) 285:2486-2497), diagnosis of the metabolic syndrome is made when three or more of the following risk factors are present:
1. Abdominal obesity, defined as waist circumference >40 inches or 102 cm in men, and >35 inches or 94 cm in women; or with regard to a Japanese ethnicity or Japanese patients defined as waist circumference ≧85 cm in men and ≧90 cm in women;
2. Triglycerides: ≧150 mg/dL
3. HDL-cholesterol <40 mg/dL in men
4. Blood pressure ≧130/85 mm Hg (SBP ≧130 or DBP ≧85)
5. Fasting blood glucose ≧110 mg/dL

The NCEP definitions have been validated (Laaksonen D E, et al. Am J Epidemiol. (2002) 156:1070-7). Triglycerides and HDL cholesterol in the blood can also be determined by standard methods in medical analysis and are described for example in Thomas L (Editor): “Labor und Diagnose”, TH-Books Verlagsgesellschaft mbH, Frankfurt/Main, 2000.

According to a commonly used definition hypertension is diagnosed if the systolic blood pressure (SBP) exceeds a value of 140 mm Hg and diastolic blood pressure (DBP) exceeds a value of 90 mm Hg. If a patient is suffering from manifest diabetes it is currently recommended that the systolic blood pressure be reduced to a level below 130 mm Hg and the diastolic blood pressure be lowered to below 80 mm Hg.

The terms “prophylactically treating” and “preventing” are used interchangeably.

### DETAILED DESCRIPTION

The aspects according to the present invention, in particular the methods and uses, refer to pyrazole-O-glucoside derivatives selected from the group of compounds (1) to (29) as defined hereinbefore and hereinafter, or prodrugs thereof, or pharmaceutically acceptable salts thereof.

Preferably all hydroxyl groups are not substituted or only the hydroxyl group connected to the carbon atom at the 6^{th} position of the β-D-glucopyranosyl group is substituted as defined. Preferred substituents are selected from among (C₁₋₃-alkyl)carbonyl, (C₁₋₆-alkyl)oxycarbonyl, phenyloxycarbonyl, benzyloxycarbonyl and benzylcarbonyl. Even more preferred substituents are selected from among acetyl, methoxycarbonyl and ethoxycarbonyl, in particular ethoxycarbonyl.

Preferred prodrugs are selected from the group consisting of
(30a) 4-(2,3-difluoro-4-methoxy-benzyl)-1-isopropyl-5-methyl-3-(6-O-methoxycarbonyl-β-D-glucopyranos-1-yloxy)-1H-pyrazole;
(30b) 4-(2,3-difluoro-4-methoxy-benzyl)-1-isopropyl-5-methyl-3-(6-O-ethoxycarbonyl-β-D-glucopyranos-1-yloxy)-1H-pyrazole;
(31a) 4-(3-fluoro-4-ethoxy-benzyl)-1-isopropyl-5-methyl-3-(6-O-methoxycarbonyl-β-D-glucopyranos-1-yloxy)-1H-pyrazole;
(31b) 4-(3-fluoro-4-ethoxy-benzyl)-1-isopropyl-5-methyl-3-(6-O-ethoxycarbonyl-β-D-glucopyranos-1-yloxy)-1H-pyrazole;
(32a) 4-(3-fluoro-4-isopropoxy-benzyl)-1-isopropyl-5-methyl-3-(6-O-methoxycarbonyl-β-D-glucopyranos-1-yloxy)-1H-pyrazole;
(32b) 4-(3-fluoro-4-isopropoxy-benzyl)-1-isopropyl-5-methyl-3-(6-O-ethoxycarbonyl-β-D-glucopyranos-1-yloxy)-1H-pyrazole;
(33a) 4-(2,5-difluoro-4-methoxy-benzyl)-1-isopropyl-5-methyl-3-(6-O-methoxycarbonyl-β-D-glucopyranos-1-yloxy)-1H-pyrazole;
(33b) 4-(2,5-difluoro-4-methoxy-benzyl)-1-isopropyl-5-methyl-3-(6-O-ethoxycarbonyl-β-D-glucopyranos-1-yloxy)-1H-pyrazole;
(34a) 4-(2-fluoro-4-isopropoxy-benzyl)-1-isopropyl-5-methyl-3-(6-O-methoxycarbonyl-β-D-glucopyranos-1-yloxy)-1H-pyrazole;
(34b) 4-(2-fluoro-4-isopropoxy-benzyl)-1-isopropyl-5-methyl-3-(6-O-ethoxycarbonyl-β-D-glucopyranos-1-yloxy)-1H-pyrazole;
(35a) 4-(2-fluoro-4-ethoxy-benzyl)-1-isopropyl-5-methyl-3-(6-O-methoxycarbonyl-β-D-glucopyranos-1-yloxy)-1H-pyrazole;
(35b) 4-(2-fluoro-4-ethoxy-benzyl)-1-isopropyl-5-methyl-3-(6-O-ethoxycarbonyl-β-D-glucopyranos-1-yloxy)-1H-pyrazole;
(36a) 4-(2-fluoro-4-methoxy-benzyl)-1-isopropyl-5-trifluoromethyl-3-(6-O-methoxycarbonyl-β-D-glucopyranos-1-yloxy)-1H-pyrazole;
(36b) 4-(2-fluoro-4-methoxy-benzyl)-1-isopropyl-5-trifluoromethyl-3-(6-O-ethoxycarbonyl-β-D-glucopyranos-1-yloxy)-1H-pyrazole;
(37a) 4-(2,6-difluoro-4-methoxy-benzyl)-1-isopropyl-5-methyl-3-(6-O-methoxycarbonyl-β-D-glucopyranos-1-yloxy)-1H-pyrazole;
(37b) 4-(2,6-difluoro-4-methoxy-benzyl)-1-isopropyl-5-methyl-3-(6-O-ethoxycarbonyl-β-D-glucopyranos-1-yloxy)-1H-pyrazole;
(38a) 4-(3,5-difluoro-4-methoxy-benzyl)-1-isopropyl-5-methyl-3-(6-O-methoxycarbonyl-β-D-glucopyranos-1-yloxy)-1H-pyrazole;
(38b) 4-(3,5-difluoro-4-methoxy-benzyl)-1-isopropyl-5-methyl-3-(6-O-ethoxycarbonyl-β-D-glucopyranos-1-yloxy)-1H-pyrazole;
(39a) 1-cyclobutyl-4-(3-fluoro-4-methyl-benzyl)-5-methyl-3-(6-O-methoxycarbonyl-β-D-glucopyranos-1-yloxy)-1H-pyrazole;
(39b) 1-cyclobutyl-4-(3-fluoro-4-methyl-benzyl)-5-methyl-3-(6-O-ethoxycarbonyl-β-D-glucopyranos-1-yloxy)-1H-pyrazole;
(40a) 1-cyclopropylmethyl-4-(3-fluoro-4-methyl-benzyl)-5-methyl-3-(6-methoxycarbonyl-β-D-glucopyranos-1-yloxy)-1H-pyrazole;
(40b) 1-cyclopropylmethyl-4-(3-fluoro-4-methyl-benzyl)-5-methyl-3-(6-ethoxycarbonyl-β-D-glucopyranos-1-yloxy)-1H-pyrazole;
(41a) 1-cyclobutyl-4-(2-fluoro-4-methoxy-benzyl)-5-methyl-3-(6-O-methoxycarbonyl-β-D-glucopyranos-1-yloxy)-1H-pyrazole;
(41b) 1-cyclobutyl-4-(2-fluoro-4-methoxy-benzyl)-5-methyl-3-(6-O-ethoxycarbonyl-β-D-glucopyranos-1-yloxy)-1H-pyrazole;
(42a) 4-(4-bromo-3-fluoro-benzyl)-1-isopropyl-5-methyl-3-(6-O-methoxycarbonyl-β-D-glucopyranos-1-yloxy)-1H-pyrazole;
(42b) 4-(4-bromo-3-fluoro-benzyl)-1-isopropyl-5-methyl-3-(6-O-ethoxycarbonyl-β-D-glucopyranos-1-yloxy)-1H-pyrazole;
(43a) 4-(2,3-difluoro-4-methyl-benzyl)-1-isopropyl-5-methyl-3-(6-O-methoxycarbonyl-β-D-glucopyranos-1-yloxy)-1H-pyrazole;
(43b) 4-(2,3-difluoro-4-methyl-benzyl)-1-isopropyl-5-methyl-3-(6-O-ethoxycarbonyl-β-D-glucopyranos-1-yloxy)-1H-pyrazole;
(44a) 4-(4-bromo-2-fluoro-benzyl)-1-isopropyl-5-methyl-3-(6-O-methoxycarbonyl-β-D-glucopyranos-1-yloxy)-1H-pyrazole;
(44b) 4-(4-bromo-2-fluoro-benzyl)-1-isopropyl-5-methyl-3-(6-O-ethoxycarbonyl-β-D-glucopyranos-1-yloxy)-1H-pyrazole;
(45a) 4-(2-fluoro-4-methyl-benzyl)-1-isopropyl-5-methyl-3-(6-O-methoxycarbonyl-β-D-glucopyranos-1-yloxy)-1H-pyrazole;
(45b) 4-(2-fluoro-4-methyl-benzyl)-1-isopropyl-5-methyl-3-(6-O-ethoxycarbonyl-β-D-glucopyranos-1-yloxy)-1H-pyrazole;
   or pharmaceutically acceptable salts thereof.

In addition further preferred prodrugs are selected from the group consisting of the compounds (46) to (63), or pharmaceutically acceptable salts thereof, as defined hereinbefore and hereinafter.

Yet further preferred prodrugs are selected from the group consisting of the compounds (64) to (73)
(64) 4-(3-fluoro-4-methoxy-benzyl)-1-isopropyl-5-methyl-3-(6-O-methoxycarbonyl-β-D-glucopyranos-1-yloxy)-1H-pyrazole;
(65) 4-(4-ethyl-benzyl)-1-isopropyl-5-trifluoromethyl-3-(6-O-methoxycarbonyl-β-D-glucopyranos-1-yloxy)-1H-pyrazole;
(66) 4-(4-bromo-benzyl)-1-isopropyl-5-trifluoromethyl-3-(6-O-methoxycarbonyl-β-D-glucopyranos-1-yloxy)-1H-pyrazole;
(67) 4-(4-ethyl-benzyl)-1-cyclobutyl-5-trifluoromethyl-3-(6-O-methoxycarbonyl-β-D-glucopyranos-1-yloxy)-1H-pyrazole;
(68) 4-(4-ethyl-benzyl)-1-(2-fluoro-1-fluoromethyl-ethyl)-5-trifluoromethyl-3-(6-O-methoxycarbonyl-β-D-glucopyranos-1-yloxy)-1H-pyrazole;
(69) 4-(3-fluoro-4-methoxy-benzyl)-1-isopropyl-5-trifluoromethyl-3-(6-O-methoxycarbonyl-β-D-glucopyranos-1-yloxy)-1H-pyrazole;
(70) 4-(3-fluoro-4-methyl-benzyl)-1-isopropyl-5-methyl-3-(6-O-methoxycarbonyl-β-D-glucopyranos-1-yloxy)-1H-pyrazole;
(71) 4-(4-isopropoxy-benzyl)-1-isopropyl-5-methyl-3-(6-O-methoxycarbonyl-β-D-glucopyranos-1-yloxy)-1H-pyrazole;
(72) 4-(2-fluoro-4-methoxy-benzyl)-1-isopropyl-5-methyl-3-(6-O-methoxycarbonyl-β-D-glucopyranos-1-yloxy)-1H-pyrazole;
(73) 4-(4-ethyl-benzyl)-1-isopropyl-5-methyl-3-(6-O-methoxycarbonyl-β-D-glucopyranos-1-yloxy)-1H-pyrazole;
   or a pharmaceutically acceptable salt thereof.

According to a first preferred embodiment the aspects according to the present invention, in particular the methods and uses, refer to
(1) 4-(2,3-difluoro-4-methoxy-benzyl)-1-isopropyl-5-methyl-3-β-D-glucopyranos-1-yloxy-1H-pyrazole;
   or a prodrug thereof wherein the hydroxyl group connected to the carbon atom at the 6^{th} position of the β-D-glucopyranosyl group is substituted with a substituent selected from among (C₁₋₃-alkyl)carbonyl, (C₁₋₆-alkyl)oxycarbonyl, phenyloxycarbonyl, benzyloxycarbonyl and benzylcarbonyl, in particular selected from among acetyl, methoxycarbonyl and ethoxycarbonyl; for example compound (30a) and (30b).

According to a second preferred embodiment the aspects according to the present invention, in particular the methods and uses, refer to
(11) 4-(2,3-difluoro-4-methyl-benzyl)-1-isopropyl-5-methyl-3-β-D-glucopyranos-1-yloxy-1H-pyrazole;
   or a prodrug thereof wherein the hydroxyl group connected to the carbon atom at the 6^{th} position of the β-D-glucopyranosyl group is substituted with a substituent selected from among (C₁₋₃-alkyl)carbonyl, (C₁₋₆-alkyl)oxycarbonyl, phenyloxycarbonyl, benzyloxycarbonyl and benzylcarbonyl, in particular selected from among acetyl, methoxycarbonyl and ethoxycarbonyl; for example compound (43a) and (43b).

According to a third preferred embodiment the aspects according to the present invention, in particular the methods and uses, refer to
(12) 4-(2-fluoro-4-methyl-benzyl)-1-isopropyl-5-methyl-3-β-D-glucopyranos-1-yloxy-1H-pyrazole;
   or a prodrug thereof wherein the hydroxyl group connected to the carbon atom at the 6^{th} position of the β-D-glucopyranosyl group is substituted with a substituent selected from among (C₁₋₃-alkyl)carbonyl, (C₁₋₆-alkyl)oxycarbonyl, phenyloxycarbonyl, benzyloxycarbonyl and benzylcarbonyl, in particular selected from among acetyl, methoxycarbonyl and ethoxycarbonyl; for example compound (45a) and (45b).

According to a fourth preferred embodiment the aspects according to the present invention, in particular the methods and uses, refer to
(16) 4-(2-fluoro-4-methoxy-benzyl)-1-isopropyl-5-methyl-3-β-D-glucopyranos-1-yloxy-1H-pyrazole;
   or a prodrug thereof wherein the hydroxyl group connected to the carbon atom at the 6^{th} position of the β-D-glucopyranosyl group is substituted with a substituent selected from among (C₁₋₃-alkyl)carbonyl, (C₁₋₆-alkyl)oxycarbonyl, phenyloxycarbonyl, benzyloxycarbonyl and benzylcarbonyl, in particular selected from among acetyl, methoxycarbonyl and ethoxycarbonyl; for example compound (47) and (72).

According to a fifth preferred embodiment the aspects according to the present invention, in particular the methods and uses, refer to
(20) 4-(2-fluoro-4-ethoxy-benzyl)-1-isopropyl-5-methyl-3-β-D-glucopyranos-1-yloxy-1H-pyrazole;
   or a prodrug thereof wherein the hydroxyl group connected to the carbon atom at the 6^{th} position of the β-D-glucopyranosyl group is substituted with a substituent selected from among (C₁₋₃-alkyl)carbonyl, (C₁₋₆-alkyl)oxycarbonyl, phenyloxycarbonyl, benzyloxycarbonyl and benzylcarbonyl, in particular selected from among acetyl, methoxycarbonyl and ethoxycarbonyl; for example compound (35a) and (35b).

According to a sixth preferred embodiment the aspects according to the present invention, in particular the methods and uses, refer to
(26) 4-(3-fluoro-4-methyl-benzyl)-1-isopropyl-5-methyl-3-(3-β-D-glucopyranos-1-yloxy-1H-pyrazole;
   or a prodrug thereof wherein the hydroxyl group connected to the carbon atom at the 6^{th} position of the β-D-glucopyranosyl group is substituted with a substituent selected from among (C₁₋₃-alkyl)carbonyl, (C₁₋₆-alkyl)oxycarbonyl, phenyloxycarbonyl, benzyloxycarbonyl and benzylcarbonyl, in particular selected from among acetyl, methoxycarbonyl and ethoxycarbonyl; for example compound (46) and (70).

According to a seventh preferred embodiment the aspects according to the present invention, in particular the methods and uses, refer to
(28) 4-(3-fluoro-4-methoxy-benzyl)-1-isopropyl-5-methyl-3-β-D-glucopyranos-1-yloxy-1H-pyrazole;
   or a prodrug thereof wherein the hydroxyl group connected to the carbon atom at the 6^{th} position of the β-D-glucopyranosyl group is substituted with a substituent selected from among (C₁₋₃-alkyl)carbonyl, (C₁₋₆-alkyl)oxycarbonyl, phenyloxycarbonyl, benzyloxycarbonyl and benzylcarbonyl, in particular selected from among acetyl, methoxycarbonyl and ethoxycarbonyl; for example compound (62) and (64).

When this invention refers to patients requiring treatment or prevention, it relates primarily to treatment and prevention in humans, but the active substance may also be used accordingly in veterinary medicine on mammals.

Within the scope of the present invention the pharmaceutical composition comprising a pyrazole-O-glucoside derivative selected from the group of compounds (1) to (29), or a prodrug thereof, or a pharmaceutically acceptable salt thereof, is preferably administered orally. Other forms of administration are possible and described hereinafter. Furthermore the treatment and/or prophylaxis, in the following called therapy, according to this invention is preferably a monotherapy, i.e. during the time of the therapy preferably no other antidiabetic drug other than the compound according to this invention is given to the patient.

As described hereinbefore by the administration of a pyrazole-O-glucoside derivative according to this invention, or a prodrug or pharmaceutically acceptable salt thereof, excessive blood glucose is excreted through the urine of the patient, so that no gain in weight or even a reduction of the weight may result. Therefore a treatment or prophylaxis according to this invention is advantageously suitable in those patients in need of such treatment or prophylaxis who are diagnosed of one or more of the conditions selected from the group consisting of overweight, class I obesity, class II obesity, class III obesity, visceral obesity and abdominal obesity or for those individuals in which a weight increase is contraindicated.

It was found that a pyrazole-O-glucoside derivative according to this invention, or a prodrug or pharmaceutically acceptable salt thereof, exhibits a very good efficacy with regard to glycemic control, in particular in view of a reduction of fasting plasma glucose, postprandial plasma glucose and/or glycosylated hemoglobin (HbA1c). By administering a pyrazole-O-glucoside derivative according to this invention, or a prodrug or pharmaceutically acceptable salt thereof, a reduction of HbA1c equal to or greater than preferably 0.5%, even more preferably equal to or greater than 1.0% can be achieved and the reduction is particularly in the range from 1.0% to 1.5%.

Furthermore the method according to this invention is advantageously applicable in those patients who show one, two or more of the following conditions:
(a) a fasting blood glucose or serum glucose concentration greater than 110 mg/dL, in particular greater than 125 mg/dL;
(b) a postprandial plasma glucose equal to or greater than 140 mg/dL;
(c) an HbA1c value equal to or greater than 6.5%, in particular equal to or greater than 8.0%.

The present invention also discloses the use of a pharmaceutical composition for improving glycemic control in patients having type 2 diabetes or showing first signs of prediabetes. Thus, the invention also includes diabetes prevention. If therefore a pyrazole-O-glucoside derivative according to this invention, or a prodrug or pharmaceutically acceptable salt thereof, is used immediately to improve the glycemic control as soon as one of the above-mentioned signs of prediabetes is present, the onset of manifest type 2 diabetes mellitus can be delayed or prevented.

Furthermore the pyrazole-O-glucoside derivative according to this invention, or a prodrug or pharmaceutically acceptable salt thereof, is particularly suitable in the treatment of patients with insulin dependency, i.e. in patients who are treated or otherwise would be treated or need treatment with an insulin or a derivative of insulin or a substitute of insulin or a formulation comprising an insulin or a derivative or substitute thereof. These patients include patients with diabetes type 2 and patients with diabetes type 1.

It can be found that by using a pyrazole-O-glucoside derivative according to this invention, or a prodrug or pharmaceutically acceptable salt thereof, an improvement of the glycemic control can be achieved even in those patients who have insufficient glycemic control in particular despite treatment with one or more antidiabetic drugs, for example despite maximal tolerated dose of oral monotherapy with either metformin or an antidiabetic of the class of sulphonylureas. A maximal tolerated dose with regard to metformin is for example 850 mg three times a day or any equivalent thereof. In the scope of the present invention the term “insufficient glycemic control” means a condition wherein patients show HbA1c values above 6.5%, in particular above 8%.

Therefore according to a preferred embodiment of the present invention there is provided a method for improving glycemic control and/or for reducing of fasting plasma glucose, of postprandial plasma glucose and/or of glycosylated hemoglobin HbA1c in a patient in need thereof who is diagnosed with impaired glucose tolerance, with insulin resistance, with metabolic syndrome and/or with type 2 or type 1 diabetes mellitus characterized in that a pharmaceutical composition comprising a pyrazole-O-glucoside derivative according to this invention, or a prodrug or pharmaceutically acceptable salt thereof, is administered.

It was found that the lowering of the blood glucose level by the administration of a pyrazole-O-glucoside derivative according to this invention, or a prodrug or pharmaceutically acceptable salt thereof, is insulin-independent. Therefore a pyrazole-O-glucoside derivative according to this invention, or a prodrug or pharmaceutically acceptable salt thereof, is particularly suitable in the treatment of patients who are diagnosed having one or more of the following conditions
insulin resistance,
hyperinsulinemia,
pre-diabetes,
type 2 diabetes mellitus, particular having a late stage type 2 diabetes mellitus,
type 1 diabetes mellitus.

Furthermore a pyrazole-O-glucoside derivative according to this invention, or a prodrug or pharmaceutically acceptable salt thereof, is particularly suitable in the treatment of patients who are diagnosed having one or more of the following conditions
(a) obesity (including class I, II and/or III obesity), visceral obesity and/or abdominal obesity,
(b) triglyceride blood level ≧150 mg/dL,
(c) HDL-cholesterol blood level <40 mg/dL in female patients and <50 mg/dL in male patients,
(d) a systolic blood pressure ≧130 mm Hg and a diastolic blood pressure ≧85 mm Hg,
(e) a fasting blood glucose level ≧110 mg/dL.

It is assumed that patients diagnosed with impaired glucose tolerance, with insulin resistance and/or with metabolic syndrome suffer from an increased risk of developing a cardiovascular disease, such as for example myocardial infarction, coronary heart disease, heart insufficiency, thromboembolic events. A glycemic control according to this invention may result in a reduction of the cardiovascular risks.

Pyrazole-O-glucoside derivatives according to this invention, or prodrugs or pharmaceutically acceptable salts thereof, exhibit a good safety profile. Therefore a treatment or prophylaxis according to this invention is advantageous possible in those patients for which the treatment with other antidiabetic drugs, such as for example metformin, is contraindicated and/or who have an intolerance against such drugs at therapeutic doses. In particular a treatment or prophylaxis according to this invention is advantageous possible in those patients showing or having an increased risk for one or more of the following disorders: renal insufficiency or diseases, cardiac diseases, cardiac failure, hepatic diseases, pulmonal diseases, catabolytic states and/or danger of lactate acidosis, or female patients being pregnant or during lactation.

Furthermore it could be found that the administration of a pyrazole-O-glucoside derivative according to this invention, or a prodrug or pharmaceutically acceptable salt thereof, results in no or in a low risk of hypoglycemia. Therefore a treatment or prophylaxis according to this invention is also advantageously possible in those patients showing or having an increased risk for hypoglycemia.

Pyrazole-O-glucoside derivatives according to this invention, or prodrugs or pharmaceutically acceptable salts thereof, are particularly suitable in the long term treatment or prophylaxis of the diseases and/or conditions as described hereinbefore and hereinafter, in particular in the long term glycemic control in patients with type 2 diabetes mellitus.

The term “long term” as used hereinbefore and hereinafter indicates a treatment of or administration in a patient within a period of time longer than 12 weeks, preferably longer than 25 weeks, even more preferably longer than 1 year.

Therefore a particularly preferred embodiment of the present invention provides a method for oral therapy, preferably oral monotherapy, for improvement, especially long term improvement, of glycemic control in patients with type 2 diabetes mellitus, especially in patients with late stage type 2 diabetes mellitus, in particular in patients additionally diagnosed of overweight, obesity (including class I, class II and/or class III obesity), visceral obesity and/or abdominal obesity.

It will be appreciated that the amount of the pyrazole-O-glucoside derivative according to this invention, or the prodrug or pharmaceutically acceptable salt thereof, to be administered to the patient and required for use in treatment or prophylaxis according to the present invention will vary with the route of administration, the nature and severity of the condition for which treatment or prophylaxis is required, the age, weight and condition of the patient, concomitant medication and will be ultimately at the discretion of the attendant physician. In general however the pyrazole-O-glucoside derivative according to this invention, or the prodrug or pharmaceutically acceptable salt thereof, is included in the pharmaceutical composition or dosage form in an amount sufficient to improve glycemic control in the patient to be treated.

The pharmaceutical composition to be administered to the patient according to a method as described hereinbefore and hereinafter preferably comprises an amount in the range from 1 mg to 1000 mg, even more preferably from 10 to 500 mg, most preferably from 50 to 500 mg of a pyrazole-O-glucoside derivative according to this invention, or a prodrug or pharmaceutically acceptable salt thereof, per day with respect to an adult patient. The above specified amounts are especially preferred for oral administration. An example of a suitable pharmaceutical composition according to this invention is a tablet for oral administration comprising 200 mg of 4-(2-fluoro-4-methoxy-benzyl)-1-isopropyl-5-methyl-3-β-D-glucopyranos-1-yloxy-1H-pyrazole.

The desired dose of the pharmaceutical composition according to this invention may conveniently be presented in a single dose once daily or as divided dose administered at appropriate intervals, for example as two, three or more doses per day.

The pharmaceutical composition may be formulated for oral, rectal, nasal, topical (including buccal and sublingual), transdermal, vaginal or parenteral (including intramuscular, sub-cutaneous and intravenous) administration in liquid or solid form or in a form suitable for administration by inhalation or insufflation. The formulations may, where appropriate, be conveniently presented in discrete dosage units and may be prepared by any of the methods well known in the art of pharmacy. All methods include the step of bringing into association the active compound with liquid carriers or finely divided solid carriers or both and then, if necessary, shaping the product into the desired formulation.

The pharmaceutical composition may be formulated in the form of tablets, granules, fine granules, powders, capsules, caplets, soft capsules, pills, oral solutions, syrups, dry syrups, chewable tablets, troches, effervescent tablets, drops, suspension, fast dissolving tablets, oral fast-dispersing tablets, etc.

The pharmaceutical composition preferably comprises one or more pharmaceutical acceptable carriers which must be “acceptable” in the sense of being compatible with the other ingredients of the formulation and not deleterious to the recipient thereof.

Pharmaceutical compositions suitable for oral administration may conveniently be presented as discrete units such as capsules, including soft gelatin capsules, cachets or tablets each containing a predetermined amount of the active ingredient; as a powder or granules; as a solution, a suspension or as an emulsion, for example as syrups, elixirs or self-emulsifying delivery systems (SEDDS). The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The pharmaceutical composition according to the invention may also be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use.

Pharmaceutical compositions suitable for rectal administration wherein the carrier is a solid are most preferably presented as unit dose suppositories. Suitable carriers include cocoa butter and other materials commonly used in the art, and the suppositories may be conveniently formed by admixture of the active compound(s) with the softened or melted carrier(s) followed by chilling and shaping in moulds.

Examples of pharmaceutically acceptable carriers are known to the one skilled in the art.

Methods for the manufacture of pyrazole-O-glucoside derivatives according to this invention and of prodrugs thereof are known to the one skilled in the art. Advantageously the compounds according to this invention can be prepared using synthetic methods as described in the literature, in particular as described in the EP 1 338 603 A1, EP 1 389 621 A1, WO 04/014932, WO 04/018491, WO 04/019958, WO 04/031203, WO 04/050122 and WO 03/020737. Preferred methods for the synthesis of the compounds according to this invention are described in the examples.

When the compounds according to this invention can form salts thereof, the salts should be pharmaceutically acceptable. Pharmaceutically acceptable salts include such as salts of inorganic acid like hydrochloric acid, sulfuric acid and phosphoric acid; salts of organic carboxylic acid like oxalic acid, acetic acid, citric acid, malic acid, benzoic acid, maleic acid, fumaric acid, tartaric acid, succinic acid and glutamic acid and salts of organic sulfonic acid like methanesulfonic acid and p-toluenesulfonic acid. The salts can be formed by combining the compounds of this invention and an acid in the appropriate amount and ratio in a solvent and decomposer. They can be also obtained by the cation or anion exchange from the form of other salts.

The compounds according to this invention include solvates such as hydrates and alcohol adducts.

The biological properties of the new compounds may be investigated as it is described for example in EP 1 338 603 A1, in particular with regard to the inhibiting activity on renal brush border membrane glucose uptake and to the activity on rat's sugar urine excretion. Furthermore the following tests may be applied:

The ability of the substances to inhibit the SGLT-2 activity may be demonstrated in a test set-up in which a CHO-K1 cell line (ATCC No. CCL 61) or alternatively an HEK293 cell line (ATCC No. CRL-1573), which is stably transfected with an expression vector pZeoSV (Invitrogen, EMBL accession number L36849), which contains the cDNA for the coding sequence of the human sodium glucose cotransporter 2 (Genbank Acc. No.NM_{—}003041) (CHO-hSGLT2 or HEK-hSGLT2). These cell lines transport ¹⁴C-labelled alpha-methyl-glucopyranoside (¹⁴C-AMG, Amersham) into the interior of the cell in sodium-dependent manner.

The SGLT-2 assay is carried out as follows:

CHO-hSGLT2 cells are cultivated in Ham's F12 Medium (BioWhittaker) with 10% foetal calf serum and 250 μg/ml zeocin (Invitrogen), and HEK293-hSGLT2 cells are cultivated in DMEM medium with 10% foetal calf serum and 250 μg/ml zeocin (Invitrogen).

The cells are detached from the culture flasks by washing twice with PBS and subsequently treating with trypsin/EDTA. After the addition of cell culture medium the cells are centrifuged, resuspended in culture medium and counted in a Casy cell counter. Then 40,000 cells per well are seeded into a white, 96-well plate coated with poly-D-lysine and incubated overnight at 37° C., 5% CO₂. The cells are washed twice with 250 μl of assay buffer (Hanks Balanced Salt Solution, 137 mM NaCl, 5.4 mM KCl, 2.8 mM CaCl₂, 1.2 mM MgSO₄ and 10 mM HEPES (pH 7.4), 50 μg/ml of gentamycin). 250 μl of assay buffer and 5 μl of test compound are then added to each well and the plate is incubated for a further 15 minutes in the incubator. 5 μl of 10% DMSO are used as the negative control. The reaction is started by adding 5 μl of ¹⁴C-AMG (0.05 μCi) to each well. After 2 hours incubation at 37° C., 5% CO₂, the cells are washed again with 250 μl of PBS (20° C.) and then lysed by the addition of 25 μl of 0.1 N NaOH (5 min. at 37° C.). 200 μl of MicroScint20 (Packard) are added to each well and incubation is continued for a further 20 min at 37° C. After this incubation the radioactivity of the ¹⁴C-AMG absorbed is measured in a Topcount (Packard) using a ¹⁴C scintillation program.

To determine the selectivity with respect to human SGLT1 an analogous test is set up in which the cDNA for hSGLT1 (Genbank Acc. No. NM000343) instead of hSGLT2 cDNA is expressed in CHO-K1 or HEK293 cells.

In the foregoing and following text, H atoms of hydroxyl groups are not explicitly shown in every case in structural formulae. The Examples that follow are intended to illustrate the present invention without restricting it.

### EXAMPLES

The following abbreviations are used above and hereinafter:

| | |
|---|---|
| Bn | benzyl |
| Bu | butyl |
| DCM | dichloromethane |
| DMF | dimethylformamide |
| Et | ethyl |
| EtOAc | ethyl acetate |
| iPr | iso-propyl |
| i. vac. | in vacuo |
| Me | methyl |
| Ph | phenyl |
| RT | ambient temperature (approx. 20° C.) |
| THF | tetrahydrofuran |

Preparation of Starting Materials:

### Example I

### 2-Fluoro-4-hydroxy-benzaldehyde

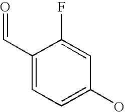

To a −70° C. solution of 2-fluoro-4-methoxy-benzaldehyde (19.1 g, 120 mmol) in CH₂Cl₂ (100 mL) was added boron tribromide in CH₂Cl₂ (1 M, 160 mL, 160 mmol). After stirring the reaction solution at −68° C. for 45 min the cooling bath was removed, and the solution was further stirred at room temperature over night. The reaction solution was poured into ice water and stirred for 30 min. The formed precipitate was separated, washed with CH₂Cl₂, and dissolved in EtOAc. The resultant EtOAc phase was washed with water and dried over MgSO₄. After evaporation of the solvent the residue was washed with little CH₂Cl₂ and dried in vacuo to give the product as a beige solid.

Yield: 14.5 g (86%) ESI-MS: m/z=139 [M−H]⁻

### Example II

### 4-Benzyloxy-3-fluoro-benzaldehyde

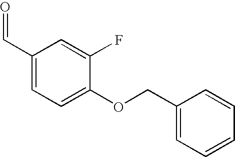

To a suspension of 4-hydroxy-3-fluoro-benzaldehyde (10.0 g, 70 mmol) and potassium carbonate (10.2 g, 74 mmol) in DMF (60 mL) was added dropwise benzyl bromide (8.7 mL, 74 mmol). The mixture was stirred at ambient temperature for 48 h and subsequently quenched with ice water. The mixture was further diluted with water, and the precipitate was separated by filtration. The precipitate was washed with water and dissolved in ethyl acetate. The organic solution was washed with brine, dried over sodium sulfate, and the solvent was removed in vacuo.

Yield: 16.0 g (99%) ESI-MS: m/z=231 [M+H]⁺

In an analogous manner the following compounds can be obtained:

### (1) 4-Benzyloxy-2-fluoro-benzaldehyde

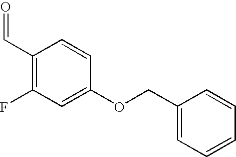

ESI-MS: m/z=253 [M+Na]⁺

### (2) 2-Chloro-4-methoxy-1-methyl-benzene

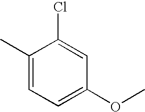

The procedure above was followed except for benzyl bromide methyl iodide was employed as the electrophile.

ESI-MS: m/z=156/158 [M]⁺ (chlorine)

### Example III

### 2,5-Difluoro-4-methoxy-benzaldehyde

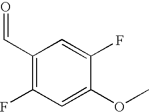

To a −65° C. solution of 1-bromo-2,5-difluoro-4-methoxy-benzene (25.0 g, 0.11 mol) in THF (150 mL) and Et₂O (250 mL) under Ar was added dropwise n-BuLi in hexane (1.6 M, 70 mL, 0.11 mol). After stirring the solution at −65° C. for 45 min, DMF (10 mL, 0.13 mol) was added slowly. The solution was warmed up in the cooling bath to room temperature over night and then diluted with Et₂O (500 mL). The resultant organic solution was washed with brine, dried over MgSO₄, and the solvent was removed in vacuo. The residue was recrystallized from iPr₂O to give the product as yellow crystals.

Yield: 6.7 g (35%) R_{f} 0.63 (silica gel, petrol ether/EtOAc 1:1)

In an analogous manner the following compounds can be obtained:

### (1) 2,6-Difluoro-4-methoxy-benzaldehyde

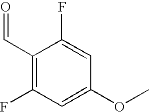

ESI-MS: m/z=173 [M+H]⁺

### (2) 3,5-Difluoro-4-methoxy-benzoic acid

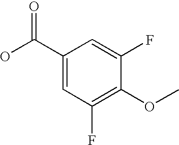

The procedure above was followed except for the quenching of the aryllithium compound with crushed dry ice (CO₂) instead of DMF.

ESI-MS: m/z=187 [M−H]⁻

### Example IV

### (4-Benzyloxy-3-fluoro-phenyl)-methanol

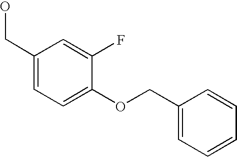

To a suspension of sodium borohydride (3.4 g, 90 mmol) in THF (60 mL) was added a solution of 4-benzyloxy-3-fluoro-benzaldehyde (16.1 g, 70 mmol) in THF (60 mL). After stirring at ambient temperature over night, the reaction mixture was quenched by the addition of ice water. The mixture was acidified with aqueous HCl (4 M) and extracted with Et₂O. The combined organic phases were washed with aqueous NaHCO₃ solution and dried over sodium sulfate. After removal of the solvent, the product was yielded.

Yield: 16.2 g (100%) ESI-MS: m/z=215 [M−OH]⁺

In an analogous manner the following compounds can be prepared:

### (1) (2,5-Difluoro-4-methoxy-phenyl)-methanol

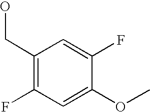

ESI-MS: m/z=215 [M−OH]⁺

### (2) (4-Benzyloxy-2-fluoro-phenyl)-methanol

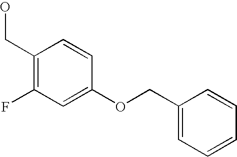

ESI-MS: m/z=232 [M]⁺

### (3) (2-Fluoro-4-methoxy-phenyl)-methanol

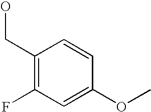

ESI-MS: m/z=139 [M−OH]⁺

### (4) (2,6-Difluoro-4-methoxy-phenyl)-methanol

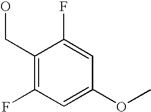

ESI-MS: m/z=157 [M−OH+H]⁺

### Example V

### (3,5-Difluoro-4-methoxy-phenyl)-methanol

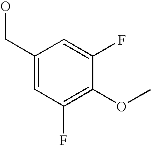

To a 20° C. suspension of lithium aluminumhydride (0.57 g, 15 mmol) in THF (50 mL) and toluene (30 mL) was added a solution of 3,5-difluoro-4-methoxy-benzoic acid (2.9 g, 15 mmol) in THF (20 mL). After stirring the reaction mixture at ambient temperature over night, ice water was added, and the resultant solution was acidified with 2 N sulfuric acid. The organic layer was separated and the aqueous extracted with EtOAc. The combined organic phases were washed with aqueous NaHCO₃ solution and brine and dried over MgSO₄. After removal of the solvent, the residue was purified by chromatography on silica gel (petrol ether/EtOAc 2:1).

Yield: 1.6 g (60%) R_{f} 0.7 (silica gel, petrol ether/EtOAc 1:1)

### Example VI

### 1-Benzyloxy-4-bromomethyl-2-fluoro-benzene

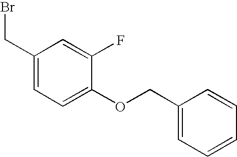

To an ice-cold solution of (4-benzyloxy-3-fluoro-phenyl)-methanol (16.7 g, 72 mmol) in diethylether (130 mL) was added phosphorous tribromide (2.8 mL, 30 mmol) at a rate such that the solution temperature did not exceed 8° C. After stirring at room temperature for 2 h, the reaction mixture was cooled in an ice-bath and quenched by the addition of ice water, ethyl acetate, and Et₂O. The organic layer was separated and washed with aqueous NaHCO₃ solution and brine. The product was yielded after evaporation of the solvent.

Yield: 20.5 g (97%) ESI-MS: m/z=294/296 [M]⁺ (bromine)

In an analogous manner the following compounds were prepared:

### (1) 1-Bromomethyl-2,5-difluoro-4-methoxy-benzene

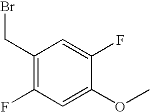

ESI-MS: m/z=236/238 [M]⁺ (bromine)

### (2) 4-Benzyloxy-1-bromomethyl-2-fluoro-benzene

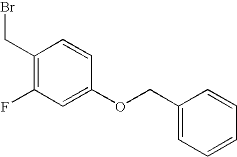

ESI-MS: m/z=294/296 [M]⁺ (bromine)

### (3) 1-Bromomethyl-2-fluoro-4-methoxy-benzene

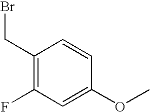

R_{f} 0.8 (silica gel, petrol ether/EtOAc 1:1)

### (4) 2-Bromomethyl-1,3-difluoro-5-methoxy-benzene

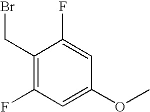

ESI-MS: m/z=236/238 [M]⁺ (bromine)

### (5) 5-Bromomethyl-1,3-difluoro-2-methoxy-benzene

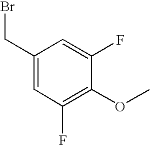

ESI-MS: m/z=236/238 [M]⁺ (bromine)

### Example VII

### 2,3-Difluoro-1-methoxy-4-methyl-benzene

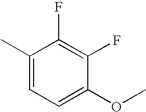

To a 20° C. solution of sodium hydroxide (14.4 g, 0.36 mol) and 2,3-difluoro-4-methyl-phenol (50.0 g, 0.35 mol) in water (160 mL) was added dropwise dimethyl sulfate (34 mL, 0.36 mol). After stirring at room temperature over night, the reaction solution was extracted with Et₂O. The ether phase was washed with 2 N NaOH solution, water, and brine and subsequently dried over MgSO₄. After removal of the solvent under reduced pressure, the product was yielded as a colorless oil.

Yield: 49.0 g (89%) ESI-MS: m/z=158 [M]⁺

### Example VIII

### 1-Bromomethyl-2,3-difluoro-4-methoxy-benzene

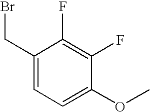

A solution of 2,3-difluoro-1-methoxy-4-methyl-benzene (39.5 g, 0.25 mol), N-bromo succinimide (44.5 g, 0.25 mol), and azobisisobutyronitrile (0.41 g, 2.5 mmol) in CCl₄ (300 mL) was stirred at reflux for 3.5 h. Then the formed succinimide was removed by filtration, and the filtrate was concentrated in vacuo. The residue was dissolved in Et₂O (200 mL) and concentrated to about 100 mL. After cooling in an ice-bath the formed precipitate was filtered off, washed with cold Et₂O, and dried in vacuo to give the product as a white solid.

Yield: 36.0 g (61%) R_{f} 0.3 (silica gel, petrol ether/EtOAc 20:1)

The following compounds can be obtained by analogy with the procedure described above:

### (1) 4-Bromomethyl-2-chloro-1-methoxy-benzene

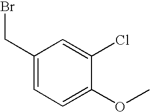

R_{f} 0.4 (silica gel, petrol ether/EtOAc 20:1)

### (2) 1-Bromomethyl-2-chloro-4-methoxy-benzene

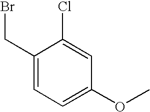

R_{f} 0.5 (silica gel, petrol ether/EtOAc 20:1)

### Example IX

### 2-(2,3-Difluoro-4-methoxy-benzyl)-3-oxo-buthylic acid ethyl ester

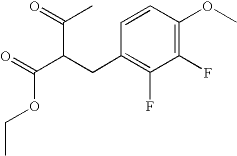

To an ice-cold suspension of sodium hydride (4.8 g, 120 mmol, 60% in mineral oil, freed from oil with pentane) in THF (140 mL) was added 3-oxo-butyric acid ethyl ester (17.2 g, 132 mmol) in THF (50 mL). After removing the ice-bath and stirring the solution at room temperature for 0.5 h, a solution of 1-methoxy-4-bromomethyl-2,3-difluoro-benzene (28.4 g, 120 mmol) in THF (60 mL) was added dropwise. After stirring the reaction mixture at reflux over night, the solvent was removed in vacuo and the residue was triturated with Et₂O (300 mL). The ether phase was washed with water and brine and dried over MgSO₄. The product was furnished as a yellow oil after evaporation of the solvent.

Yield: 35.5 g (ca. 80% pure) ESI-MS: m/z=285 [M−H]⁻

The following compounds can be obtained in an analogous manner:

### (1) 2-(4-Benzyloxy-3-fluoro-benzyl)-3-oxo-butyric acid ethyl ester

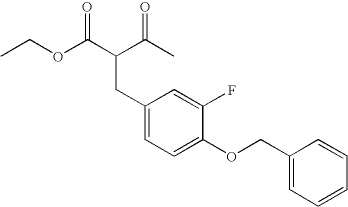

ESI-MS: m/z=345 [M+H]⁺

### (2) 2-(4-Iodo-benzyl)-3-oxo-butyric acid ethyl ester

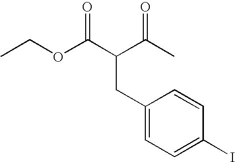

ESI-MS: m/z=345 [M−H]⁻

### (3) 2-(2,5-Difluoro-4-methoxy-benzyl)-3-oxo-butyric acid ethyl ester

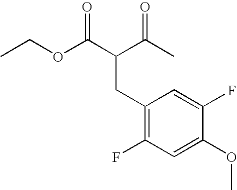

R_{f} 0.27 (silica gel, petrol ether/EtOAc 4:1)

### (4) 2-(4-Benzyloxy-2-fluoro-benzyl)-3-oxo-butyric acid ethyl ester

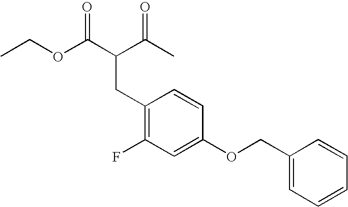

ESI-MS: m/z=343 [M−H]⁻

### (5) 2-(2,6-Difluoro-4-methoxy-benzyl)-3-oxo-butyric acid ethyl ester

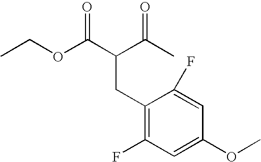

ESI-MS: m/z=287 [M+H]⁺

### (6) 2-(3,5-Difluoro-4-methoxy-benzyl)-3-oxo-butyric acid ethyl ester

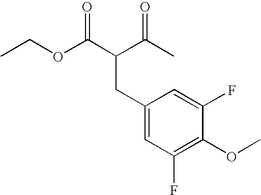

ESI-MS: m/z=287 [M+H]⁺

### (7) 2-(3-Fluoro-4-methyl-benzyl)-3-oxo-butyric acid ethyl ester

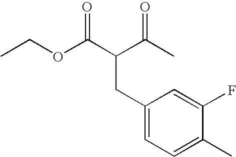

ESI-MS: m/z=253 [M+H]⁺

### (8) 2-(2-Fluoro-4-methoxy-benzyl)-3-oxo-butyric acid ethyl ester

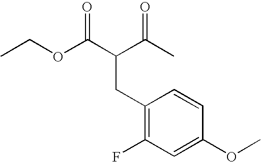

ESI-MS: m/z=269 [M+H]⁺

### (9) 2-(3-Chloro-4-methoxy-benzyl)-3-oxo-butyric acid ethyl ester

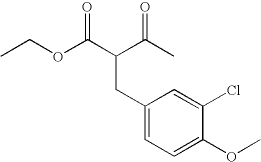

ESI-MS: m/z=283/285 [M−H]⁻(chlorine)

### (10) 2-(2-Chloro-4-methoxy-benzyl)-3-oxo-butyric acid ethyl ester

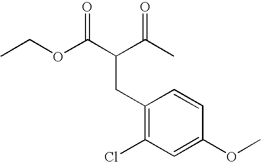

ESI-MS: m/z=285/287 [M+H]⁺ (chlorine)

### (11) 4,4,4-Trifluoro-2-(2-fluoro-4-methoxy-benzyl)-3-oxo-butyric acid ethyl ester

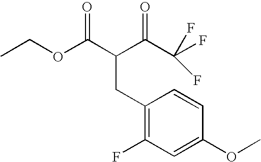

ESI-MS: m/z=321 [M−H]⁻

### Example X

### 2-(2,3-Difluoro-4-methyl-benzyl)-3-oxo-buthylic acid ethyl ester

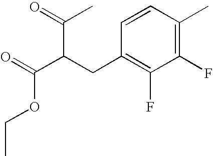

To an ice-cold solution of 3-oxo-butyric acid ethyl ester (4.17 g, 32.1 mmol) and sodium iodide (23.9 g, 160 mmol) under Ar in acetonitrile (220 mL) was added over 3 min trimethylsilyl chloride (20.2 mL, 160 mmol) followed by 2,3-difluoro-4-methyl-benzaldehyde (5.0 g, 32.1 mmol). The ice bath was removed, and the reaction mixture was stirred at room temperature for 8 h and subsequently at 60° C. for 15 h. After cooling to room temperature the reaction mixture was poured into a mixture of EtOAc (300 mL) and water (200 mL). The organic phase was separated and washed with aqueous Na₂S_{2O}₃ solution and brine and dried over Na₂SO₄. The solvent was removed under reduced pressure, and the residue was purified by silica gel chromatography (hexane/EtOAc 1:6) to give the product as a colorless oil.

Yield: 8.4 g (97%) R_{f} 0.35 (silica gel, hexane/EtOAc 5:1)

The following compounds can be obtained in an analogous manner:

### (1) 2-(4-Bromo-3-fluoro-benzyl)-3-oxo-butyric acid ethyl ester

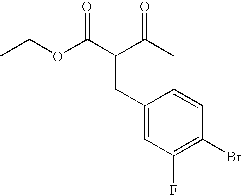

### (2) 2-(4-Bromo-2-fluoro-benzyl)-3-oxo-butyric acid ethyl ester

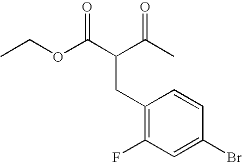

R_{f} 0.42 (silica gel, hexane/EtOAc 4:1)

### (3) 2-(2-fluoro-4-methyl-benzyl)-3-oxo-butyric acid ethyl ester

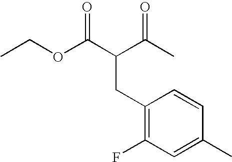

### Example Xl

### 4,4,4-Trifluoro-2-(2-fluoro-4-methoxy-benzyl)-3-methoxy-but-2-enoic acid ethyl ester

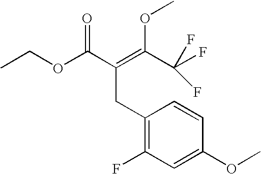

To a 20° C. mixture of 4,4,4-trifluoro-2-(2-fluoro-4-methoxy-benzyl)-3-oxo-butyric acid ethyl ester (6.35 g, 19.7 mmol) and cesium carbonate (9.5 g, 28.9 mmol) in DMF (50 mL) was dropped a solution of toluene-4-sulfonic acid methyl ester (4.5 g, 23.7 mmol) in DMF (20 mL). The reaction mixture was stirred at room temperature over night and subsequently at 60° C. for 1.5 h. After cooling to room temperature diluted phosphoric acid was added, and the resultant solution was extracted with Et₂O. The combined organic phases were washed with brine and dried over Na₂SO₄. After removal of the solvent the residue was purified by chromatography on aluminum oxide (cyclohexane/EtOAc 99:1->70:30).

Yield: 6.6 g (100%) ESI-MS: m/z=337 [M+H]⁺

### Example XII

### 4-(2,3-Difluoro-4-methoxy-benzyl)-5-methyl-1H-pyrazol-3-ol

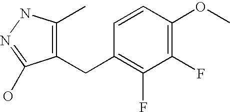

A solution of 2-(2,3-difluoro-4-methoxy-benzyl)-3-oxo-butyric acid ethyl ester (33.0 g, 0.115 mol) and hydrazine hydrate (80%, 8.0 g, 128 mmol) in EtOH (300 mL) was stirred at reflux for 2 h. After cooling in an ice-bath the precipitate was collected, washed with cold EtOH, and dried in vacuo to give the product as a white solid.

Yield: 22.5 g (70%) ESI-MS: m/z=255 [M+H]⁺

The following compounds can be obtained accordingly:

### (1) 4-(4-Benzyloxy-3-fluoro-benzyl)-5-methyl-1H-pyrazol-3-ol

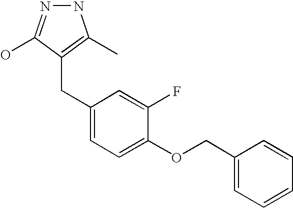

ESI-MS: m/z=313 [M+H]⁺

### (2) 4-(4-Iodo-benzyl)-5-methyl-1H-pyrazol-3-ol

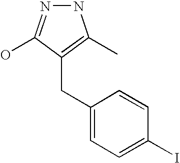

ESI-MS: m/z=315 [M+H]⁺

### (3) 4-(2,5-Difluoro-4-methoxy-benzyl)-5-methyl-1H-pyrazol-3-ol

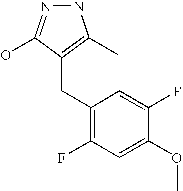

ESI-MS: m/z=255 [M+H]⁺

### (4) 4-(4-Benzyloxy-2-fluoro-benzyl)-5-methyl-1H-pyrazol-3-ol

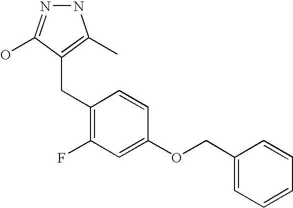

ESI-MS: m/z=313 [M+H]⁺

### (5) 4-(2,6-Difluoro-4-methoxy-benzyl)-5-methyl-1H-pyrazol-3-ol

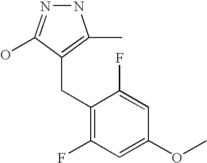

ESI-MS: m/z=255 [M+H]⁺

### (6) 4-(3,5-Difluoro-4-methoxy-benzyl)-5-methyl-1H-pyrazol-3-ol

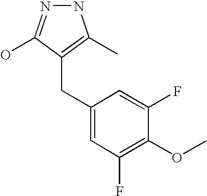

ESI-MS: m/z=255 [M+H]⁺

### (7) 4-(3-Fluoro-4-methyl-benzyl)-5-methyl-1H-pyrazol-3-ol

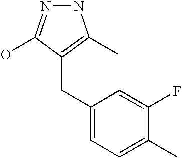

ESI-MS: m/z=221 [M+H]⁺

### (8) 4-(2-Fluoro-4-methoxy-benzyl)-5-methyl-1H-pyrazol-3-ol

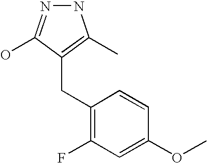

ESI-MS: m/z=237 [M+H]⁺

### (9) 4-(3-Chloro-4-methoxy-benzyl)-5-methyl-1H-pyrazol-3-ol

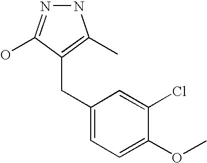

ESI-MS: m/z=253/255 [M+H]⁺ (chlorine)

### (10) 4-(2-Chloro-4-methoxy-benzyl)-5-methyl-1H-pyrazol-3-ol

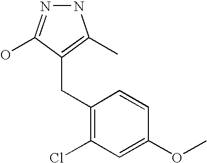

ESI-MS: m/z=253/255 [M+H]⁺ (chlorine)

### (11) 4-(2-Fluoro-4-methoxy-benzyl)-5-trifluoromethyl-1H-pyrazol-3-ol

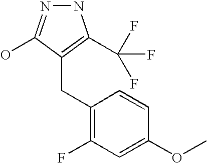

The product was prepared following the procedure above starting from 4,4,4-trifluoro-2-(2-fluoro-4-methoxy-benzyl)-3-methoxy-but-2-enoic acid ethyl ester

ESI-MS: m/z=289 [M−H]⁻

### (12) 4-(4-Bromo-3-fluoro-benzyl)-5-methyl-1H-pyrazol-3-ol

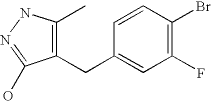

### (13) 4-(2,3-Difluoro-4-methyl-benzyl)-5-methyl-1H-pyrazol-3-ol

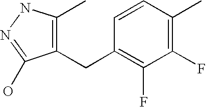

R_{f} 0.05 (silica gel, hexane/EtOAc 5:1)

### (14) 4-(4-Bromo-2-fluoro-benzyl)-5-methyl-1H-pyrazol-3-ol

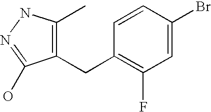

R_{f} 0.15 (silica gel, hexane/EtOAc 1:1)

### (15) 4-(2-Fluoro-4-methyl-benzyl)-5-methyl-1H-pyrazol-3-ol

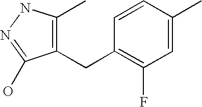

R_{f} 0.11 (silica gel, hexane/EtOAc 1:1)

### Example XIII

### 3-(tert-Butyl-dimethyl-silyloxy)-4-(2-fluoro-4-methoxy-benzyl)-5-trifluoromethyl-1H-pyrazole

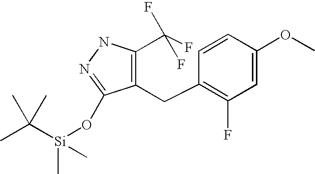

To a solution of 4-(2-fluoro-4-methoxy-benzyl)-5-trifluoromethyl-1H-pyrazol-3-ol (0.21 g, 0.72 mmol) and imidazole (8.0 g, 128 mmol) in DMF (2 mL) was added tert-butyldimethylsilylchloride (0.13 g, 0.86 mmol). After stirring at room temperature for 4 h, the solution was diluted with EtOAc and washed with water and brine. The organic phase was dried and the solvent removed.

Yield: 0.34 g (ca. 80% pure) ESI-MS: m/z=405 [M+H]⁺

### Example XIV

### 3-(tert-Butyl-dimethyl-silyloxy)-4-(2-fluoro-4-methoxy-benzyl)-1-isopropyl-5-trifluoromethyl-1H-pyrazole

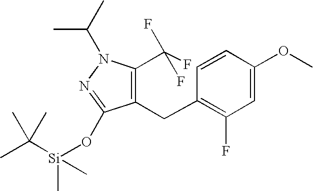

To a suspension of 3-(tert-butyl-dimethyl-silyloxy)-4-(2-fluoro-4-methoxy-benzyl)-5-trifluoromethyl-1H-pyrazole (0.27 g, 0.67 mmol) and Ph₃P (0.20 g, 0.76 mmol) in isopropanol (2 mL) was added diethyl azodicarboxylate in toluene (40%, 0.35 mL, 0.76 mmol). The solution was stirred at room temperature for 1 h and then diluted with Et₂O. The resultant solution was washed with water and aqueous NaOH solution (2 N), dried over Na₂SO₄, and the solvent was removed. The residue was purified by chromatography on silica gel (cyclohexane/EtOAc 99:1->4:1) to give the product as a colorless oil.

Yield: 0.14 g (47%) ESI-MS: m/z=447 [M+H]⁺

### Example XV

### 4-(2-Fluoro-4-methoxy-benzyl)-1-isopropyl-5-trifluoromethyl-1H-pyrazol-3-ol

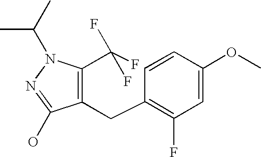

A solution of 3-(tert-butyl-dimethyl-silyloxy)-4-(2-fluoro-4-methoxy-benzyl)-1-isopropyl-5-trifluoromethyl-1H-pyrazole (0.27 g, 0.67 mmol), aqueous HCl (1 N, 1 mL, 1 mmol), MeOH (0.5 mL), and THF (12 mL) was stirred at 60° C. for 2 h. After cooling to room temperature the solution was diluted with EtOAc and washed with water and brine. The product was yielded as a white solid after drying over Na₂SO₄ and removal of the solvent in vacuo.

Yield: 0.10 g (100%) ESI-MS: m/z=333 [M+H]⁺

### Example XVI

### 4-(2,3-Difluoro-4-methoxy-benzyl)-5-methyl-3-(2,3,4,6-tetra-O-benzyl-β-D-glucopyranos-1-yloxy)-1H-pyrazole

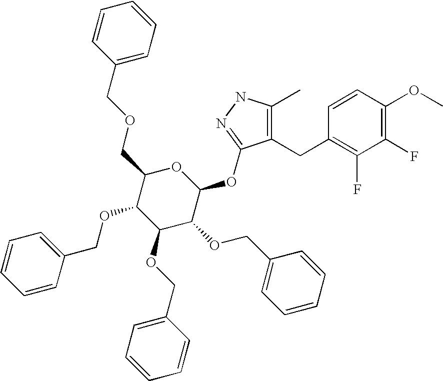

To a 0° C. solution of 4-(2,3-Difluoro-4-methoxy-benzyl)-5-methyl-1H-pyrazol-3-ol (2.14 g, 8.4 mmol), 2,3,4,6-tetra-O-benzyl-α-D-gluco-pyranose (4.54 g, 8.4 mmol), and PPh₃ (2.20 g, 8.4 mmol) in dry THF (80 mL) was added diethyl azodicarboxylate in toluene (40%, 3.85 mL, 8.4 mmol) at a rate such that the solution maintained 2-6° C. After 10 min the cooling bath was removed, and the reaction solution was stirred at room temperature over night. Then the solution was concentrated at 40° C. under reduced pressure, and the remainder was treated with Et₂O (50 mL). The ether solution was cooled to −18° C., and the forming precipitate was separated and washed with cold Et₂O. The filtrate was diluted with Et₂O and washed with aqueous NaOH solution (2 N), water, and brine. After drying over MgSO₄ and evaporation of the solvent, the residue was purified by chromatography on silica gel (cyclohexane/EtOAc 2:1->1:6). The purified product was recrystallized from iPr₂O to give the product as a white solid (<5% a anomer).

Yield: 3.10 g (48%) ESI-MS: m/z=777 [M+H]⁺

The following compounds can be obtained accordingly:

### (1) 4-(2,5-Difluoro-4-methoxy-benzyl)-5-methyl-3-(2,3,4,6-tetra-O-benzyl-β-D-glucopyranos-1-yloxy)-1H-pyrazole

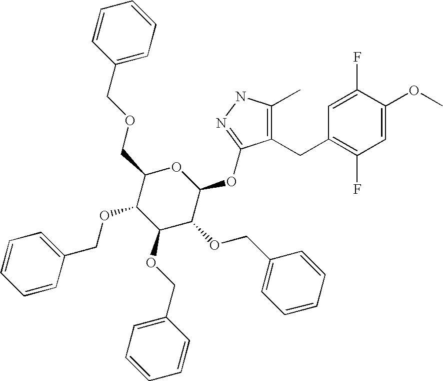

ESI-MS: m/z=777 [M+H]⁺

### (2) 4-(2-Fluoro-4-benzyloxy-benzyl)-5-methyl-3-(2,3,4,6-tetra-O-benzyl-β-D-glucopyranos-1-yloxy)-1H-pyrazole

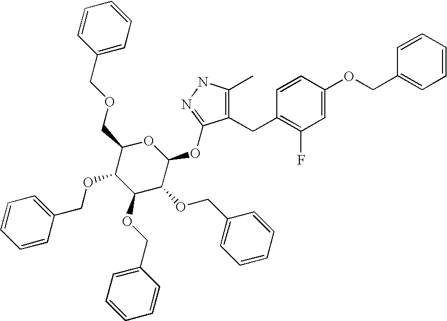

ESI-MS: m/z=835 [M+H]⁺

### (3) 4-(2,6-Difluoro-4-methoxy-benzyl)-5-methyl-3-(2,3,4,6-tetra-O-benzyl-β-D-glucopyranos-1-yloxy)-1H-pyrazole

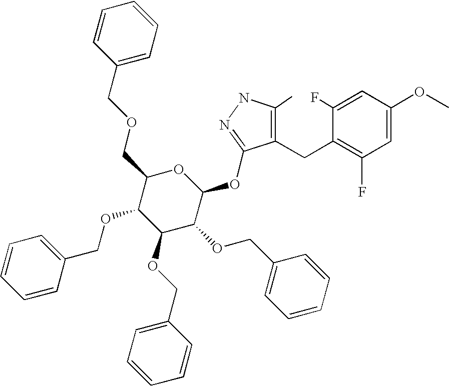

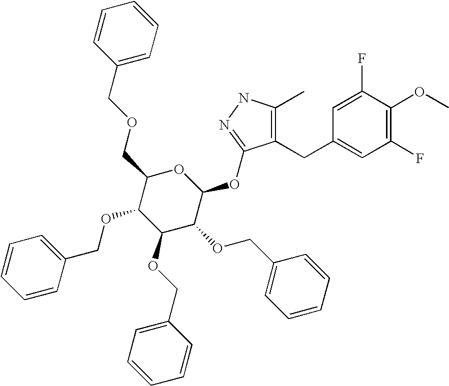

ESI-MS: m/z=777 [M+H]⁺

### (4) 4-(3,5-Difluoro-4-methoxy-benzyl)-5-methyl-3-(2,3,4,6-tetra-O-benzyl-β-D-glucopyranos-1-yloxy)-1H-pyrazole

ESI-MS: m/z=777 [M+H]⁺

### (5) 4-(3-Fluoro-4-methyl-benzyl)-5-methyl-3-(2,3,4,6-tetra-O-benzyl-β-D-glucopyranos-1-yloxy)-1H-pyrazole

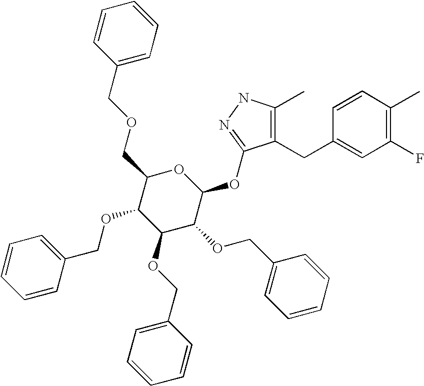

Bu₃P and 1,1′-(azodicarbonyl)-dipiperidine were used instead of Ph₃P and diethyl azodicarboxylate

ESI-MS: m/z=743 [M+H]⁺

### (6) 4-(2-Fluoro-4-methoxy-benzyl)-5-methyl-3-(2,3,4,6-tetra-O-benzyl-β-D-glucopyranos-1-yloxy)-1H-pyrazole

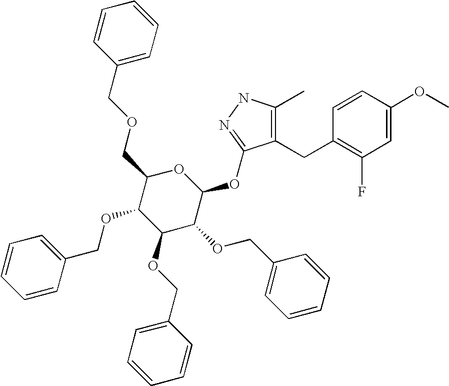

Bu₃P and 1,1′-(azodicarbonyl)-dipiperidine were used instead of Ph₃P and diethyl azodicarboxylate

ESI-MS: m/z=759 [M+H]⁺

### (7) 4-(3-Chloro-4-methoxy-benzyl)-5-methyl-3-(2,3,4,6-tetra-O-benzyl-β-D-glucopyranos-1-yloxy)-1H-pyrazole

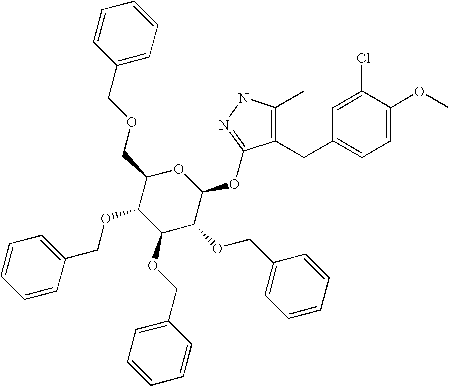

BU₃P and 1,1′-(azodicarbonyl)-dipiperidine were used instead of Ph₃P and diethyl azodicarboxylate

ESI-MS: m/z=775/777 [M+H]⁺ (chlorine)

### (8) 4-(2-Chloro-4-methoxy-benzyl)-5-methyl-3-(2,3,4,6-tetra-O-benzyl-β-D-glucopyranos-1-yloxy)-1H-pyrazole

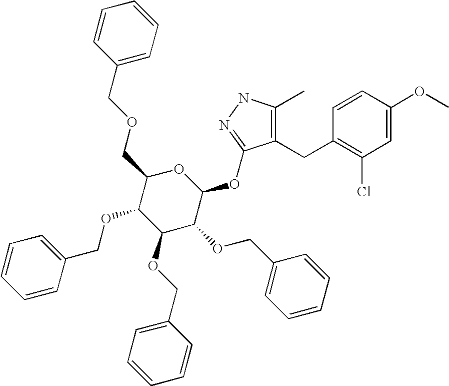

Bu₃P and 1,1′-(azodicarbonyl)-dipiperidine were used instead of Ph₃P and diethyl azodicarboxylate

ESI-MS: m/z=775/777 [M+H]⁺ (chlorine)

### (9) 4-(4-Bromo-3-fluoro-benzyl)-5-methyl-3-(2,3,4,6-tetra-O-benzyl-β-D-glucopyranos-1-yloxy)-1H-pyrazole

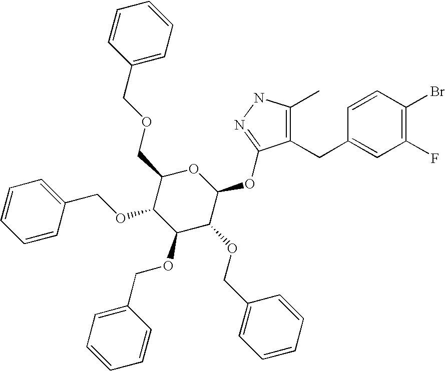

### (10) 4-(2,3-Difluoro-4-methyl-benzyl)-5-methyl-3-(2,3,4,6-tetra-O-benzyl-β-D-glucopyranos-1-yloxy)-1H-pyrazole

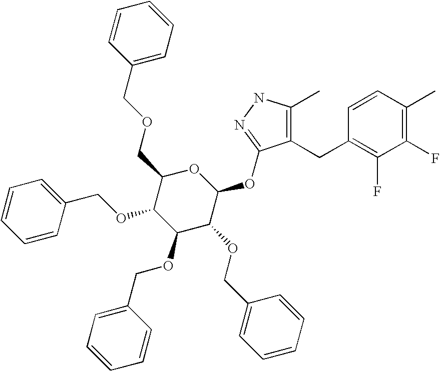

R_{f} 0.24 (silica gel, hexane/EtOAc 1:1)

### (11) 4-(2-Fluoro-4-methyl-benzyl)-5-methyl-3-(2,3,4,6-tetra-O-benzyl-β-D-glucopyranos-1-yloxy)-1H-pyrazole

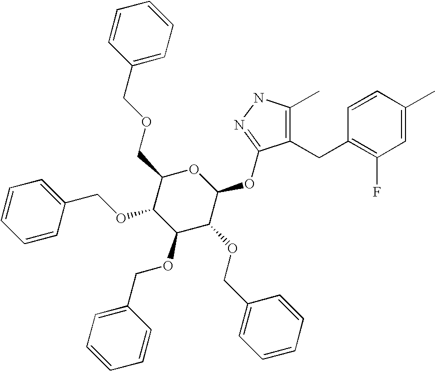

R_{f} 0.48 (silica gel, hexane/EtOAc 1:1)

### Example XVII

### 4-(4-iodo-benzyl)-5-methyl-3-(2,3,4,6-tetra-O-acetyl-β-D-glucopyranos-1-yloxy)-1H-pyrazole

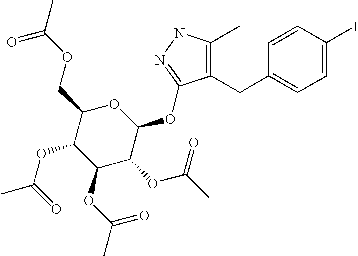

To a solution of 4-(4-iodo-benzyl)-5-methyl-1H-pyrazol-3-ol (0.70 g, 2.23 mmol) in dry THF (80 mL) was added Ag₂CO₃ (0.65 g, 2.36 mmol) followed by 2,3,4,6-tetra-O-acetyl-β-D-glucopyranos-1-ylbromide (1.00 g, 2.43 mmol). The reaction mixture was stirred at reflux in the dark over night prior to the addition of another portion of Ag₂CO₃ (0.75 g, 2.72 mmol) and 2,3,4,6-tetra-O-acetyl-β-D-glucopyranos-1-ylbromide (1.10 g, 2.68 mmol). The reaction mixture was stirred at reflux for another night and then cooled to room temperature. The mixture was filtrated, and the filtrate was concentrated in vacuo. The residue was purified by chromatography on silica gel (CH₂Cl₂/MeOH 1:0->10:1) to give the product as a white solid.

Yield: 0.40 g (28%) ESI-MS: m/z=645 [M+H]⁺

The following compounds can be obtained accordingly:

### (1) 4-(4-benzyloxy-3-fluoro-benzyl)-5-methyl-3-(2,3,4,6-tetra-O-acetyl-β-D-glucopyranos-1-yloxy)-1H-pyrazole

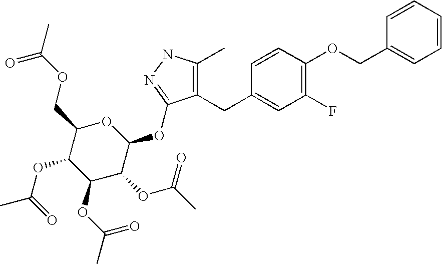

ESI-MS: m/z=643 [M+H]⁺

### (2) 4-(4-Bromo-2-fluoro -benzyl)-5-methyl-3-(2,3,4,6-tetra-O-acetyl-β-D-glucopyranos-1-yloxy)-1H-pyrazole

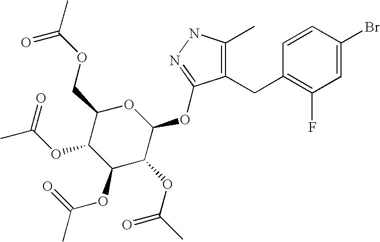

R_{f} 0.46 (silica gel, hexane/EtOAc 1:1)

### Example XVIII

### 4-(2-fluoro-4-methoxy-benzyl)-5-trifluoromethyl-3-(2,3,4,6-tetra-O-acetyl-β-D-glucopyranos-1-yloxy)-1H-pyrazole

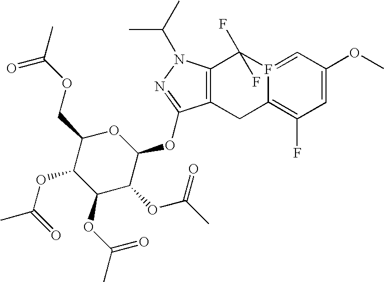

To a solution of 4-(2-Fluoro-4-methoxy-benzyl)-1-isopropyl-5-trifluoromethyl-1H-pyrazol-3-ol (1.84 g, 5.54 mmol), K₂CO₃ (7.5 g, 54.3 mmol), and nBu₃BnNCl (0.25 g, 0.8 mmol) in water (5 mL) and CH₂Cl₂ (25 mL) was added 2,3,4,6-tetra-O-acetyl-β-D-glucopyranos-1-ylbromide (3.80 g, 8.78 mmol). The reaction mixture was stirred vigorously at room temperature in the dark over night. Then CH₂Cl₂ was added and the organic layer was separated. After washing with water and 1 M phosphoric acid, the organic phase was dried over Na₂SO₄, and the solvent was removed. The residue was purified by chromatography on silica gel (cyclohexane/EtOAc 3:2->0:1).

Yield: 2.42 g (ca. 50% pure) ESI-MS: m/z=663 [M+H]⁺

### Example XIX

### 4-(2,3-Difluoro-4-methoxy-benzyl)-1-isopropyl-5-methyl-3-(2,3,4,6-tetra-O-benzyl-β-D-glucopyranos-1-yloxy)-1H-pyrazole

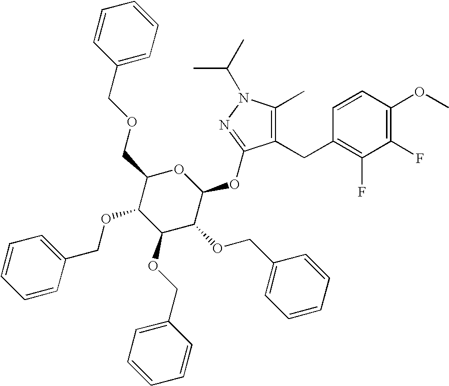

To a 20° C. mixture of 4-(2,3-difluoro-4-methoxy-benzyl)-5-methyl-3-(2,3,4,6-tetra-O-benzyl-β-D-glucopyranos-1-yloxy)-1H-pyrazole (2.90 g, 3.7 mmol) and Cs₂CO₃ (12.30 g, 37.8 mmol) in DMF (56 mL) was added isopropyl iodide (1.90 mL, 18.9 mmol). The reaction mixture was stirred at room temperature for 2.5 h. Then the reaction mixture was poured into water (300 mL), and the resultant solution was extracted with EtOAc. The combined organic extracts were washed with water and brine and dried over MgSO₄. The organic solution was concentrated at 40° C. under reduced pressure, and the residue was purified by chromatography on silica gel (cyclohexane/EtOAc 6:1->1:1).

Yield: 2.10 g (69%) ESI-MS: m/z=459 [M+H]⁺

The following compounds can be obtained accordingly:

### (1) 4-(4-benzyloxy-3-fluoro-benzyl)-1-isopropyl-5-methyl-3-(2,3,4,6-tetra-O-acetyl-β-D-glucopyranos-1-yloxy)-1H-pyrazole

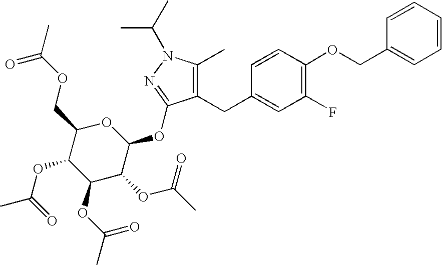

ESI-MS: m/z=685 [M+H]⁺

### (2) 4-(4-iodo-benzyl)-1-isopropyl-5-methyl-3-(2,3,4,6-tetra-O-acetyl-β-D-glucopyranos-1-yloxy)-1H-pyrazole

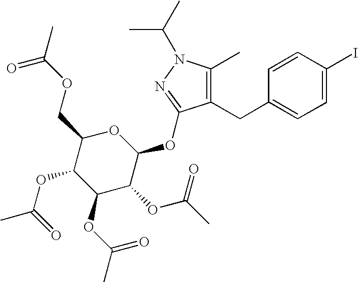

ESI-MS: m/z=687 [M+H]⁺

### (3) 4-(2,5-Difluoro-4-methoxy-benzyl)-1-isopropyl-5-methyl-3-(2,3,4,6-tetra-O-benzyl-β-D-glucopyranos-1-yloxy)-1H-pyrazole

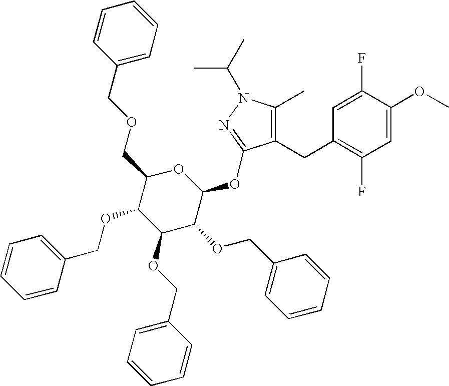

ESI-MS: m/z=819 [M+H]⁺

### (4) 4-(2-Fluoro-4-benzyloxy-benzyl)-1-isopropyl-5-methyl-3-(2,3,4,6-tetra-O-benzyl-β-D-glucopyranos-1-yloxy)-1H-pyrazole

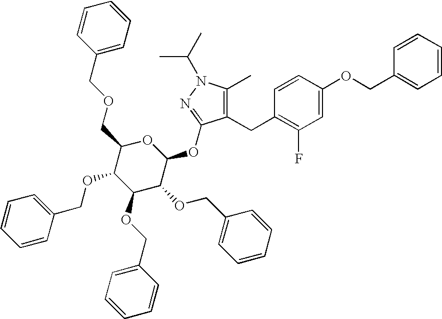

ESI-MS: m/z=877 [M+H]⁺

### (5) 4-(2,6-Difluoro-4-methoxy-benzyl)-1-isopropyl-5-methyl-3-(2,3,4,6-tetra-O-benzyl-β-D-glucopyranos-1-yloxy)-1H-pyrazole

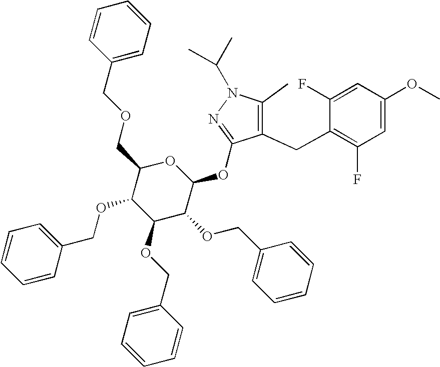

ESI-MS: m/z=819 [M+H]⁺

### (6) 4-(3,5-Difluoro-4-methoxy-benzyl)-1-isopropyl-5-methyl-3-(2,3,4,6-tetra-O-benzyl-β-D-glucopyranos-1-yloxy)-1H-pyrazole

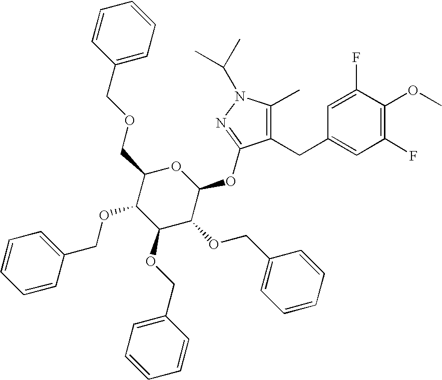

ESI-MS: m/z=819 [M+H]⁺

### (7) 1-Cyclobutyl-4-(3-fluoro-4-methyl-benzyl)-5-methyl-3-(2,3,4,6-tetra-O-benzyl-β-D-glucopyranos-1-yloxy)-1H-pyrazole

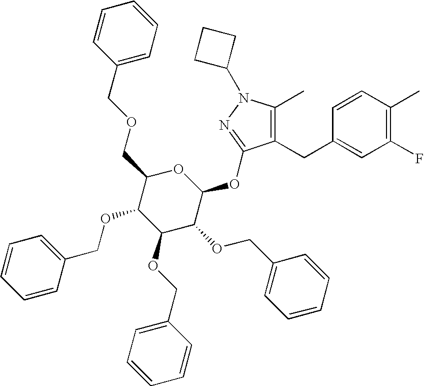

Bromo-cyclobutane was used as the electrophile instead of isopropyl iodide

ESI-MS: m/z=797 [M+H]⁺

### (8) 1-Cyclopropylmethyl-4-(3-fluoro-4-methyl-benzyl)-5-methyl-3-(2,3,4,6-tetra-O-benzyl-β-D-glucopyranos-1-yloxy)-1H-pyrazole

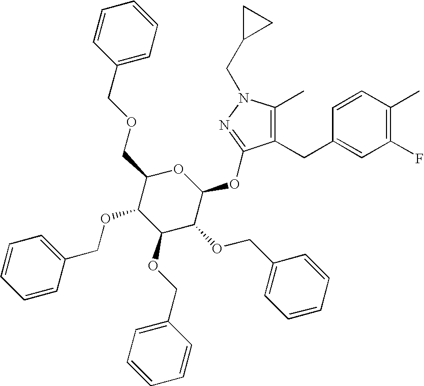

Yielded as a side product in the preparation of example XVIII (7)

ESI-MS: m/z=797 [M+H]⁺

### (9) 1-Cyclobutyl-4-(2-fluoro-4-methoxy-benzyl)-5-methyl-3-(2,3,4,6-tetra-O-benzyl-β-D-glucopyranos-1-yloxy)-1H-pyrazole

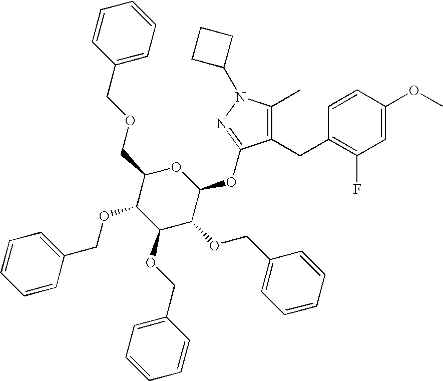

Bromo-cyclobutane was used as the electrophile instead of isopropyl iodide

ESI-MS: m/z=813 [M+H]⁺

### (10) 4-(3-Chloro-4-methoxy-benzyl)-1-isopropyl-5-methyl-3-(2,3,4,6-tetra-O-benzyl-β-D-glucopyranos-1-yloxy)-1H-pyrazole

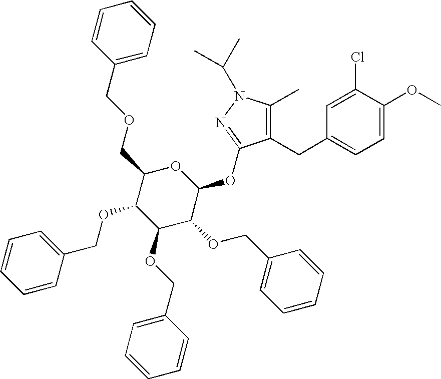

The reaction is preferably carried out with potassium hexamethyldisilazide as the base in toluene and THF

ESI-MS: m/z=817/819 [M+H]⁺ (chlorine)

### (11) 4-(2-Chloro-4-methoxy-benzyl)-1-isopropyl-5-methyl-3-(2,3,4,6-tetra-O-benzyl-β-D-glucopyranos-1-yloxy)-1H-pyrazole

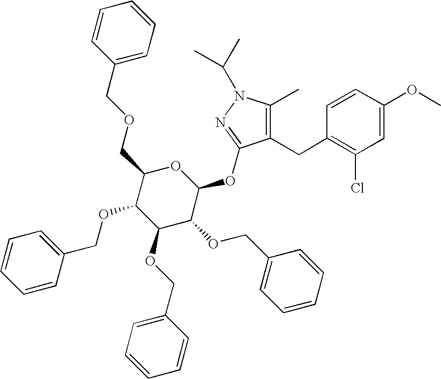

The reaction is preferably carried out with potassium hexamethyldisilazide as the base in toluene and THF

ESI-MS: m/z=817/819 [M+H]⁺ (chlorine)

### (12) 4-(4-Bromo-3-fluoro-benzyl)-1-isopropyl-5-methyl-3-(2,3,4,6-tetra-O-benzyl-β-D-glucopyranos-1-yloxy)-1H-pyrazole

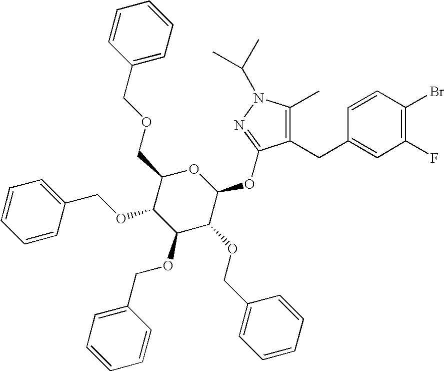

### (13) 4-(2,3-Difluoro-4-methyl-benzyl)-1-isopropyl-5-methyl-3-(2,3,4,6-tetra-O-benzyl-β-D-glucopyranos-1-yloxy)-1H-pyrazole

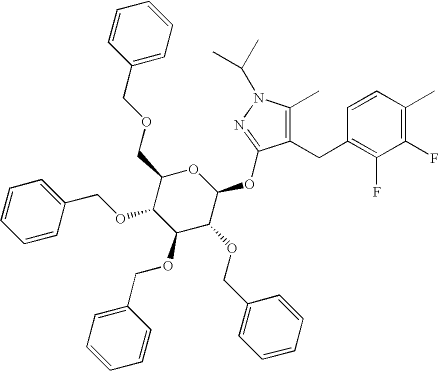

R_{f} 0.65 (silica gel, hexane/EtOAc 1:1)

### (14) 4-(4-Bromo-2-fluoro-benzyl)-1-isopropyl-5-methyl-3-(2,3,4,6-tetra-O-acetyl-β-D-glucopyranos-1-yloxy)-1H-pyrazole

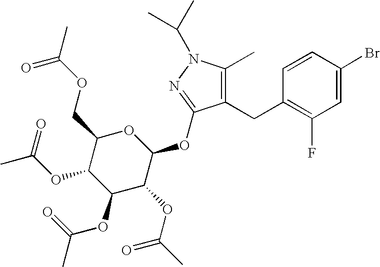

R_{f} 0.50 (silica gel, hexane/EtOAc 1:1)

### (15) 4-(2-Fluoro-4-methyl-benzyl)-1-isopropyl-5-methyl-3-(2,3,4,6-tetra-O-benzyl-β-D-glucopyranos-1-yloxy)-1H-pyrazole

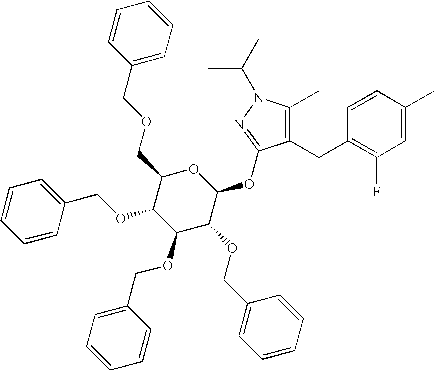

R_{f} 0.53 (silica gel, hexane/EtOAc 4:1)

### Example XX

### 4-(3-fluoro-4-hydroxy-benzyl)-1-isopropyl-5-methyl-3-(2,3,4,6-tetra-O-acetyl-β-D-glucopyranos-1-yloxy)-1H-pyrazole

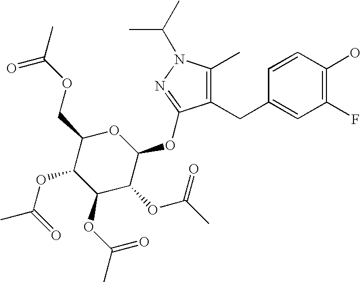

A mixture of 4-(4-benzyloxy-3-fluoro-benzyl)-1-isopropyl-5-methyl-3-(2,3,4,6-tetra-O-acetyl-β-D-glucopyranos-1-yloxy)-1H-pyrazole (0.26 g, 0.38 mmol) and 10% Pd on carbon (0.05 g) in EtOAc (10 mL) was stirred at room temperature under hydrogen atmosphere (3 bar). After 3 h the catalyst was separated by filtration, and the solvent was removed under reduced pressure. The residue was dissolved in Et₂O, filtered over Celite®, and concentrated in vacuo.

Yield: 0.22 g (97%) ESI-MS: m/z=595 [M+H]⁺

### Example XXI

### 4-(3-Fluoro-4-ethoxy-benzyl)-1-isopropryl-5-methyl-3-(2,3,4,6-tetra-O-acetyl-β-D-glucopyranos-1-yloxy)-1H-pyrazole

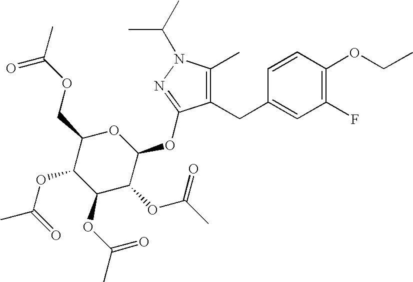

To a suspension of 4-(3-fluoro-4-hydroxy-benzyl)-1-isopropyl-5-methyl-3-(2,3,4,6-tetra-O-acetyl-β-D-glucopyranos-1-yloxy)-1H-pyrazole (0.22 g, 0.37 mmol) and cesium carbonate (0.31 g, 0.40 mmol) in DMF (3 mL) was added ethyl bromide (30 μL, 0.40 mmol). After stirring at ambient temperature for 5 h, the mixture was poured into a mixture of EtOAc and phosphoric acid (0.1 M). The organic phase was separated, washed with aqueous NaHCO₃ solution and brine, and dried over Na₂SO₄. The organic solution was concentrated, and the residue was purified by silica gel chromatography (petrol ether/EtOAc 1:1).

Yield: 0.18 g (78%) ESI-MS: m/z=623 [M+H]⁺

The following compound can be obtained accordingly:

### (1) 4-(3-Fluoro-4-isopropoxy-benzyl)-1-isopropyl-5-methyl-3-(2,3,4,6-tetra-O-acetyl-β-D-glucopyranos-1-yloxy)-1H-pyrazole

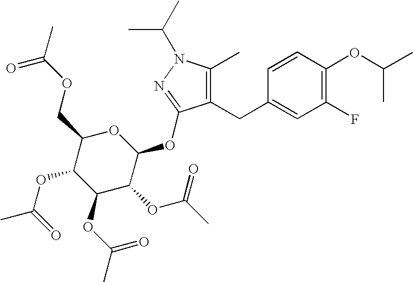

ESI-MS: m/z=637 [M+H]⁺

### Example XXII

### 4-(2-fluoro-4-hydroxy-benzyl)-1-isopropyl-5-methyl-3-β-D-glucopyranos-1-yloxy-1H-pyrazole

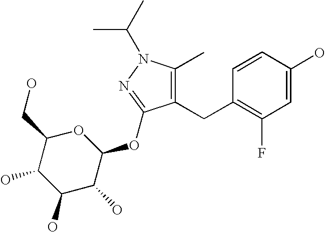

A mixture of 4-(2-fluoro-4-benzyloxy-benzyl)-1-isopropyl-5-methyl-3-(2,3,4,6-tetra-O-benzyl-β-D-glucopyranos-1-yloxy)-1H-pyrazole (2.0 g, 2.3 mmol) and 20% Pd on carbon (1.0 g) in EtOH (70 mL) was stirred at room temperature under hydrogen atmosphere (50 psi). After 2 h the catalyst was separated by filtration, and the solvent was removed under reduced pressure. The residue was purified by silica gel chromatography (CH₂Cl₂/MeOH 10:1->3:1).

Yield: 0.69 g (71%) ESI-MS: m/z=427 [M+H]⁺

### Example XXIII

### 4-(4-Trimethylsilylethinyl-benzyl)-1-isopropyl-5-methyl-3-(2,3,4,6-tetra-O-acetyl-β-D-glucopyranos-1-yloxy)-1H-pyrazole

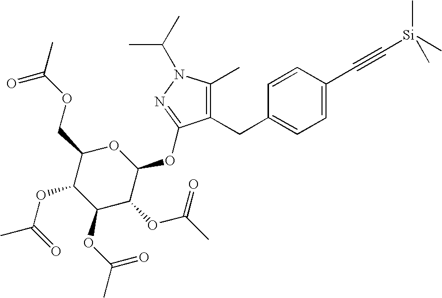

To a degassed solution of 4-(4-iodo-benzyl)-1-isopropyl-5-methyl-3-(2,3,4,6-tetra-O-acetyl-β-D-glucopyranos-1-yloxy)-1H-pyrazole (0.31 g, 0.45 mmol) in DMF (5 mL) under Ar was added in the given order NEt₃ (0.2 mL, 1.43 mmol), Cul (0.02 g, 0.11 mmol), (Ph₃P)₂PdCl₂ (0.05 g, 0.07 mmol), and trimethylsilylacetylen (0.10 mL, 0.69 mmol). The reaction mixture was stirred at 90° C. for 3.5 h. After cooling to room temperature EtOAc was added, and the resultant solution was washed with aqueous NaHCO₃ solution and dried over Na₂SO₄. The solvent was evaporated, and the residue was purified by chromatography on silica gel (cyclohexane/EtOAc 9:1->1:1) to give the product as a yellow oil.

Yield: 0.18 g (64%) ESI-MS: m/z=657 [M+H]⁺

Preparation of Products

### Example 1

### (1) 4-(2,3-Difluoro-4-methoxy-benzyl)-1-isopropyl-5-methyl-3-β-D-qlucopyranos-1-yloxy-1H-pyrazole

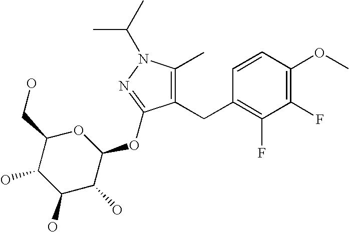

A mixture of 4-(2,3-difluoro-4-methoxy-benzyl)-1-isopropyl-5-methyl-3-(2,3,4,6-tetra-O-benzyl-β-D-glucopyranos-1-yloxy)-1H-pyrazole (1.80 g, 2.2 mmol) and 20% Pd on carbon (1 g) in EtOH (50 mL) was stirred at room temperature under hydrogen atmosphere (50 psi). After 2.5 h the catalyst was separated by filtration, and the solvent was removed under reduced pressure. The residue was purified by chromatography on silica gel (DCM/MeOH 1:0->4:1) to afford the product as a white solid.

Yield: 0.48 g (48%) ESI-MS: m/z=459 [M+H]⁺

The following compounds can be obtained accordingly:

### (2) 4-(2,5-Difluoro-4-methoxy-benzyl)-1-isopropyl-5-methyl-3-β-D-glucopyranos-1-yloxy-1H-pyrazole

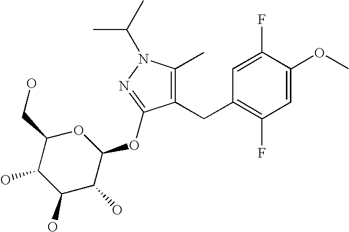

ESI-MS: m/z=459 [M+H]⁺

### (3) 4-(2,6-Difluoro-4-methoxy-benzyl)-1-isopropyl-5-methyl-3-β-D-glucopyranos-1-yloxy-1H-pyrazole

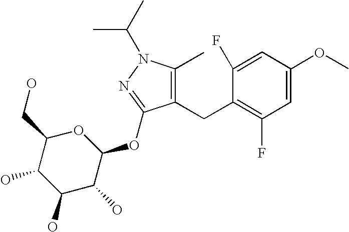

ESI-MS: m/z=459 [M+H]⁺

### (4) 4-(3,5-Difluoro-4-methoxy-benzyl)-1-isopropyl-5-methyl-3-β-D-glucopyranos-1-yloxy-1H-pyrazole

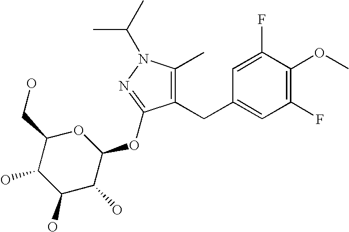

ESI-MS: m/z=459 [M+H]⁺

### (5) 1-Cyclobutyl-4-(3-fluoro-4-methyl-benzyl)-5-methyl-3-β-D-glucopyranos-1-yloxy-1H-pyrazole

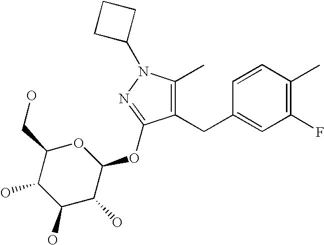

ESI-MS: m/z=437 [M+H]⁺

### (6) 1-Cyclopropylmethyl-4-(3-fluoro-4-methyl-benzyl)-5-methyl-3-β-D-glucopyranos-1-yloxy-1H-pyrazole

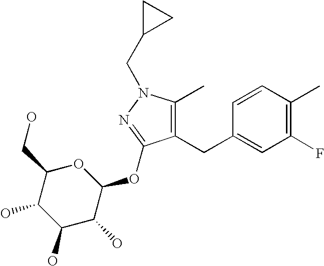

ESI-MS: m/z=437 [M+H]⁺

### (7) 1-Cyclobutyl-4-(2-fluoro-4-methoxy-benzyl)-5-methyl-3-β-D-glucopyranos-1-yloxy-1H-pyrazole

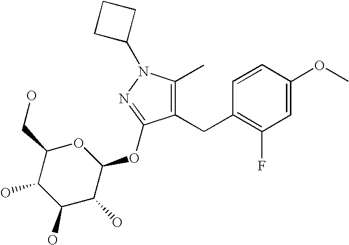

ESI-MS: m/z=453 [M+H]⁺

### (8) 4-(3-Chloro-4-methoxy-benzyl)-1-isopropyl-5-methyl-3-β-D-glucopyranos-1-yloxy-1H-pyrazole

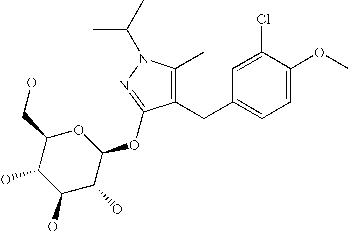

ESI-MS: m/z=457/459 [M+H]⁺ (chlorine)

### (9) 4-(2-Chloro-4-methoxy-benzyl)-1-isopropyl-5-methyl-3-β-D-glucopyranos-1-yloxy-1H-pyrazole

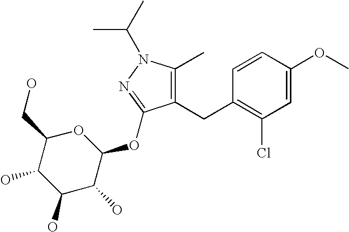

ESI-MS: m/z=457/459 [M+H]⁺ (chlorine)

### (10) 4-(4-Bromo-3-fluoro-benzyl)-1-isopropyl-5-methyl-3-β-D-glucopyranos-1-yloxy-1H-pyrazole

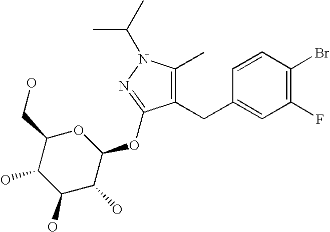

### (11) 4-(2,3-Difluoro-4-methyl-benzyl)-1-isopropyl-5-methyl-3-β-D-glucopyranos-1-yloxy-1H-pyrazole

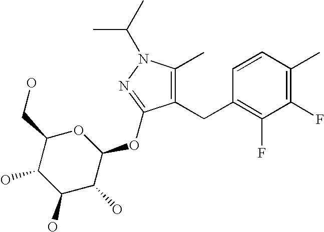

R_{f} 0.24 (silica gel, CHCl₃/MeOH 9:1)

### (12) 4-(2-Fluoro-4-methyl-benzyl)-1-isopropyl-5-methyl-3-β-D-glucopyranos-1-yloxy-1H-pyrazole

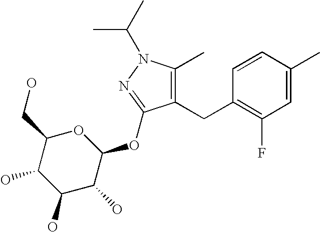

R_{f} 0.24 (silica gel, CH₂Cl₂/MeOH 9:1)

### Example 2

### (13) 4-(3-Fluoro-4-ethoxy-benzyl)-1-isopropyl-5-methyl-3-β-D-glucopyranos-1-yloxy-1H-pyrazole

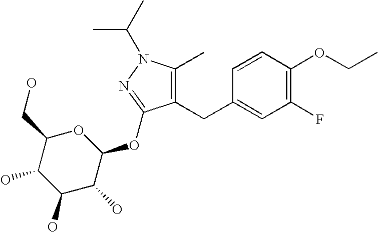

To an ice-cold solution of 4-(4-ethoxy-3-fluoro-benzyl)-1-isopropyl-5-methyl-3-(2,3,4,6-tetra-O-acetyl-β-D-glucopyranos-1-yloxy)-1H-pyrazole (0.17 g, 0.27 mmol) in MeOH (1 mL) and THF (1.5 mL) was added aqueous LiOH solution (1 M, 1.25 mL). The solution was stirred in the ice-bath for 1 h and then diluted with EtOAc and water. The organic phase was separated, washed with water and brine, and dried over Na₂SO₄. The solvent was removed, and the residue was dried in vacuo to give the product as a white foam.

Yield: 0.12 g (95%) ESI-MS: m/z=455 [M+H]⁺

The following compounds can be obtained accordingly:

### (14) 4-(4-ethinyl-benzyl)-1-isopropyl-5-methyl-3-β-D-glucopyranos-1-yloxy-1H-pyrazole

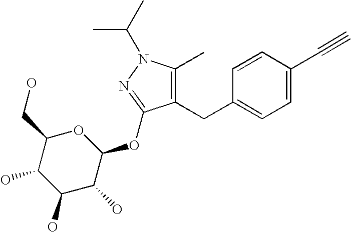

4-(4-Trimethylsilyl-ethinyl-benzyl)-1-isopropyl-5-methyl-3-(2,3,4,6-tetra-O-acetyl-β-D-glucopyranos-1-yloxy)-1H-pyrazole was subjected to the reaction conditions described above.

ESI-MS: m/z=417 [M+H]⁺

### (15) 4-(3-Fluoro-4-isopropxy-benzyl)-1-isopropyl-5-methyl-3-β-D-glucopyranos-1-yloxy-1H-pyrazole

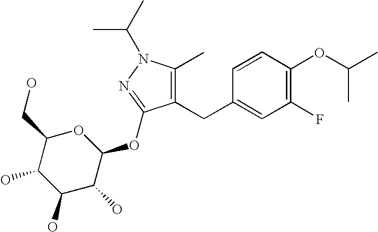

ESI-MS: m/z=469 [M+H]⁺

### (16) 4-(2-fluoro-4-methoxy-benzyl)-1-isopropyl-5-methyl-3-β-D-glucopyranos-1-yloxy-1H-pyrazole

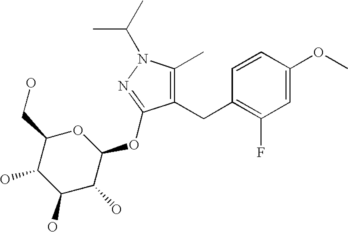

### (17) 4-(2-fluoro-4-methoxy-benzyl)-1-isopropyl-5-trifluoromethyl-3-β-D-glucopyranos-1-yloxy-1H-pyrazole

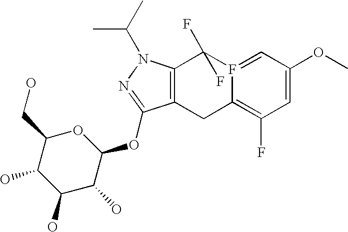 ESI-MS: m/z=495 [M₊H]⁺

### (18) 4-(4-Bromo-2-fluoro-benzyl)-1-isopropyl-5-methyl-3-β-D-glucopyranos-1-yloxy-1H-pyrazole

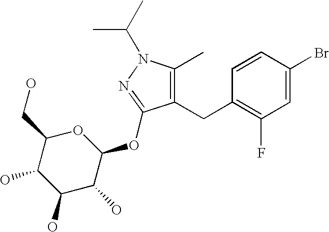 R_{f} 0.29 (silica gel, CH₂Cl₂/MeOh 9:1)

### Example 3

### (19) 4-(2-Fluoro-4-isopropoxy-benzyl)-1-isopropyl-5-methyl-3-β-D-glucopyranos-1-yloxy-1H-pyrazole

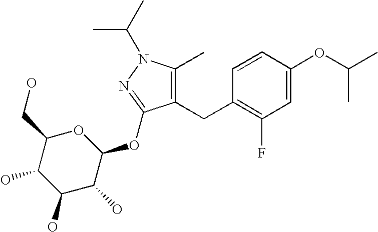

To a suspension of 4-(2-fluoro-4-hydroxy-benzyl)-1-isopropyl-5-methyl-3-β-D-glucopyranos-1-yloxy-1H-pyrazole (0.20 g, 0.47 mmol) and cesium carbonate (0.16 g, 0.50 mmol) in DMF (3.5 mL) was added isopropyl iodide (52 μL, 0.50 mmol). After stirring the mixture at ambient temperature over night, another portion of cesium carbonate (0.10 g) and isopropyl iodide (30 μL) were added. After stirring another 24 h at room temperature, the mixture was diluted with EtOAc, phosphoric acid (0.1 M), and brine. The organic phase was separated, washed with brine, and dried over Na₂SO₄. The organic solution was concentrated, and the residue was purified by silica gel chromatography (DCM/MeOH 10:1) to deliver the product as a white foam.

Yield: 0.16 g (73%) ESI-MS: m/z=469 [M+H]⁺

The following compound can be obtained accordingly:

### (20) 4-(2-Fluoro-4-ethoxy-benzyl)-1-isopropyl-5-methyl-3-β-D-glucopyranos-1-yloxy-1H-pyrazole

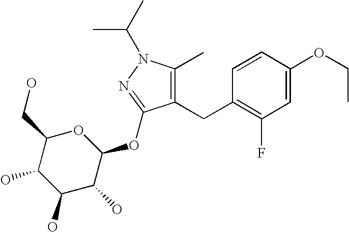

ESI-MS: m/z=455 [M+H]⁺

The compounds (21) to (29) can be obtained by methods as described in this application or in the literature.

### Example 4

### (30a) 4-(2,3-Difluoro-4-methoxy-benzyl)-1-isopronyl-5-methyl-3-(6-O-methoxycarbonyl-β-D-glucopyranos-1-yloxy)-1H-pyrazole

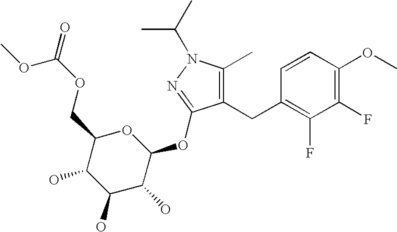

To an ice-cold solution of 4-(2,3-difluoro-4-methoxy-benzyl)-1-isopropyl-5-methyl-3-β-D-glucopyranos-1-yloxy-1H-pyrazole (0.23 g, 0.50 mmol) in 2,4,6-trimethylpyridine (0.7 mL) was added methyl chloroformate (42 μL, 0.55 mmol). The reaction solution was warmed up in the ice-bath to room temperature and stirred over night. Then the solution was diluted with Et₂O, washed with aqueous HCl (1 M) and brine, and dried over MgSO₄. The solvent was evaporated, and the residue was purified by chromatography on silica gel (DCM/MeOH 25:1->3:1) to afford the product as a white solid.

Yield: 0.15 g (56%) ESI-MS: m/z=517 [M+H]⁺

The following compounds can be obtained accordingly:

### (31a) 4-(3-Fluoro-4-ethoxy-benzyl)-1-isopropyl-5-methyl-3-(6-O-methoxycarbonyl-β-D-glucopyranos-1-yloxy)-1H-pyrazole

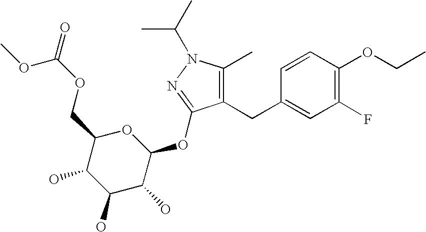

ESI-MS: m/z=513 [M+H]⁺

### (32a) 4-(3-Fluoro-4-isopropxy-benzyl)-1-isopropyl-5-methyl-3-(6-O-methoxycarbonyl-β-D-glucopyranos-1-yloxy)-1H-pyrazole

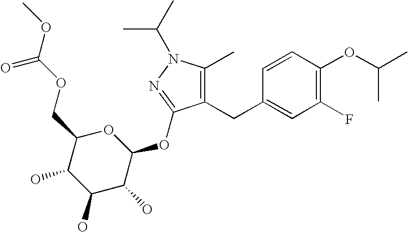

ESI-MS: m/z=527 [M+H]⁺

### (33a) 4-(2,5-Difluoro-4-methoxy-benzyl)-1-isopropyl-5-methyl-3-(6-O-methoxycarbonyl-β-D-glucopyranos-1-yloxy)-1H-pyrazole

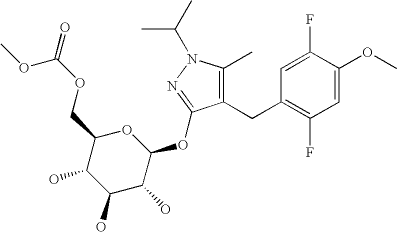

ESI-MS: m/z=517 [M+H]⁺

### (34a) 4-(2-Fluoro-4-isopropoxy-benzyl)-1-isopropyl-5-methyl-3-(6-O-methoxycarbonyl-β-D-glucopyranos-1-yloxy)-1H-pyrazole

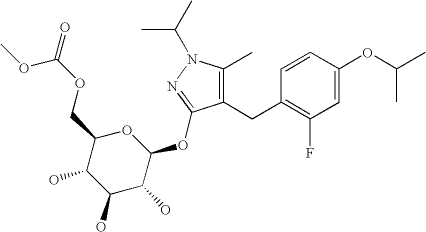

ESI-MS: m/z=527 [M+H]⁺

### (35a) 4-(2-Fluoro-4-ethoxy-benzyl)-1-isopropyl-5-methyl-3-(6-O-methoxycarbonyl-β-D-glucopyranos-1-yloxy)-1H-pyrazole

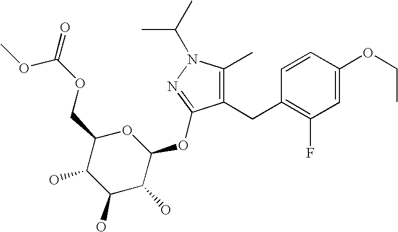

ESI-MS: m/z=513 [M+H]⁺

### (36a) 4-(2-fluoro-4-methoxy-benzyl)-1-isopropyl-5-trifluoromethyl-3-(6-O-methoxycarbonyl-β-D-glucopyranos-1-yloxy)-1H-pyrazole

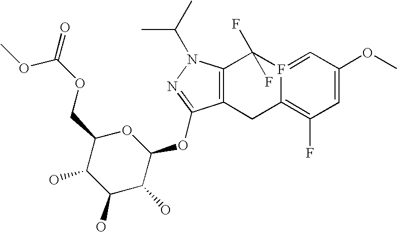

ESI-MS: m/z=553 [M+H]⁺

### (37a) 4-(2,6-Difluoro-4-methoxy-benzyl)-1-isopropyl-5-methyl-3-(6-O-methoxycarbonyl-β-D-glucopyranos-1-yloxy)-1H-pyrazole

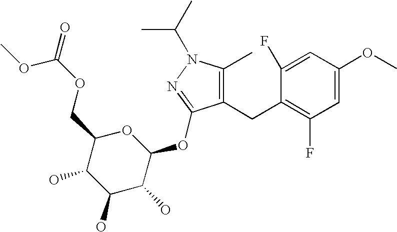

ESI-MS: m/z=517 [M+H]⁺

### (38a) 4-(3,5-Difluoro-4-methoxy-benzyl)-1-isopropyl-5-methyl-3-(6-O-methoxycarbonyl-β-D-glucopyranos-1-yloxy)-1H-pyrazole

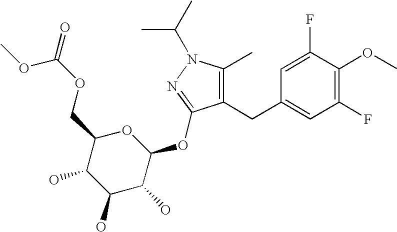

ESI-MS: m/z=517 [M+H]⁺

### (39a) 1-Cyclobutyl-4-(3-fluoro-4-methyl-benzyl)-5-methyl-3-(6-O-methoxycarbonyl-β-D-glucopyranos-1-yloxy)-1H-pyrazole

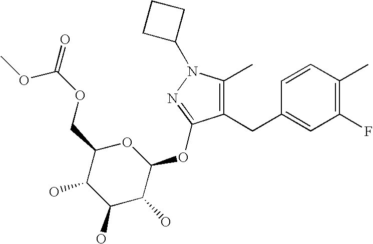

ESI-MS: m/z=495 [M+H]⁺

### (40a) 1-Cyclopropylmethyl-4-(3-fluoro-4-methyl-benzyl)-5-methyl-3-(6-methoxycarbonyl-β-D-glucopyranos-1-yloxy)-1H-pyrazole

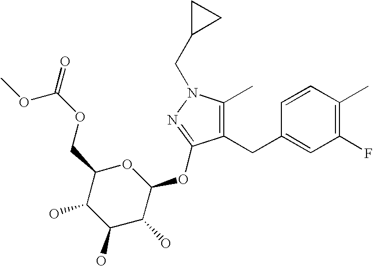

ESI-MS: m/z=495 [M+H]⁺

### (41 a) 1-Cyclobutyl-4-(2-fluoro-4-methoxy-benzyl)-5-methyl-3-(6-O-methoxycarbonyl-β-D-glucopyranos-1-yloxy)-1H-pyrazole

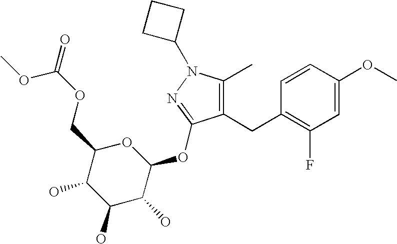

ESI-MS: m/z=511 [M+H]⁺

### (42a) 4-(4-Bromo-3-fluoro-benzyl)-1-isopropyl-5-methyl-3-(6-O-methoxycarbonyl-β-D-glucopyranos-1-yloxy)-1H-pyrazole

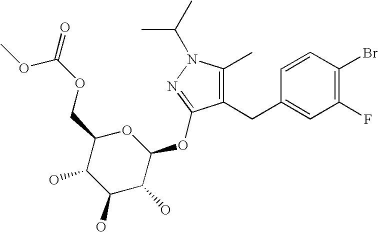

### (43a) 4-(2,3-Difluoro-4-methyl-benzyl)-1-isopropyl-5-methyl-3-(6-O-methoxycarbonyl-β-D-glucopyranos-1-yloxy)-1H-pyrazole

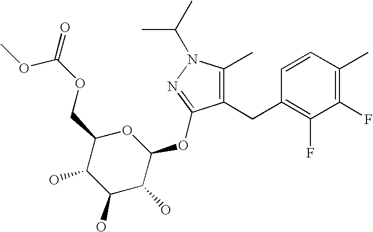

R_{f} 0.49 (silica gel, CHCl₃/MeOH 10:1)

### (44a) 4-(4-Bromo-2-fluoro-benzyl)-1-isopropyl-5-methyl-3-(6-O-methoxycarbonyl-β-D-glucopyranos-1-yloxy)-1H-pyrazole

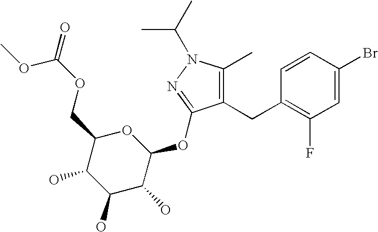

R_{f} 0.39 (silica gel, CH₂Cl₂/MeOH 19:1)

### (45a) 4-(2-Fluoro-4-methyl-benzyl)-1-isopropyl-5-methyl-3-(6-O-methoxycarbonyl-β-D-glucopyranos-1-yloxy)-1H-pyrazole

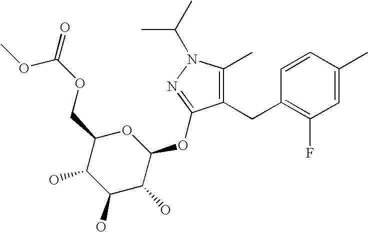

R_{f} 0.62 (silica gel, CH₂Cl₂/MeOH 9:1)

The compounds (30b), (31b), (32b), (33b), (34b), (35b), (36b), (37b), (38b), (39b), (40b), (41b), (42b), (43b), (44b) and (45b) are obtained analogously.

### Example 5

### (46) 4-(3-Fluoro-4-methyl-benzyl)-1-isopropyl-5-methyl-3-(6-O-ethoxycarbonyl-β-D-glucopyranos-1-yloxy)-1H-pyrazole

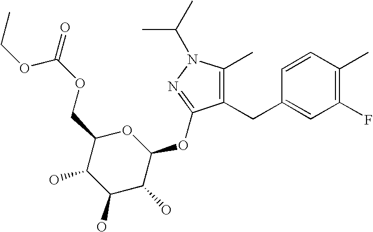

To an ice-cold solution of 4-(3-fluoro-4-methyl-benzyl)-1-isopropyl-5-methyl-3-β-D-glucopyranos-1-yloxy-1H-pyrazole (0.30 g, 0.70 mmol) in 2,4,6-trimethylpyridine (1 mL) was added methyl chloroformate (76 μL, 0.80 mmol). The reaction solution was warmed up in the ice-bath to room temperature and stirred over night. Then the solution was diluted with Et₂O, washed with aqueous HCl (1 M) and brine, and dried over MgSO₄. The solvent was evaporated and the residue was purified by chromatography on silica gel (DCM/MeOH 25:1->3:1) to afford the product as a white solid.

Yield: 0.23 g (66%) ESI-MS: m/z=497 [M+H]⁺

The following compounds can be obtained by analogy with the procedure described above:

### (47) 4-(2-Fluoro-4-methoxy-benzyl)-1-isopropyl-5-methyl-3-(6-O-ethoxycarbonyl-β-D-glucopyranos-1-yloxy)-1H-pyrazole

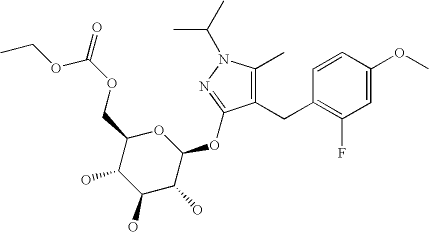

ESI-MS: m/z=513 [M+H]⁺

### (48) 4-(2-Fluoro-4-methoxy-benzyl)-1-isopropyl-5-methyl-3-(6-O-isobutyloxycarbonyl-β-D-glucopyranos-1-yloxy)-1H-pyrazole

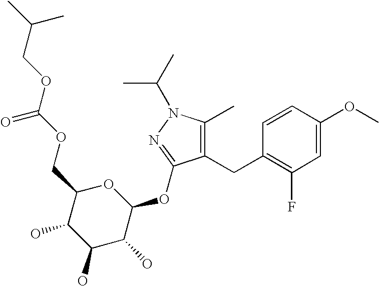

ESI-MS: m/z=541 [M+H]⁺

### (49) 4-(2-Fluoro-4-methoxy-benzyl)-1-isopropyl-5-methyl-3-(6-O-hex-1-yloxycarbonyl-β-D-glucopyranos-1-yloxy)-1H-pyrazole

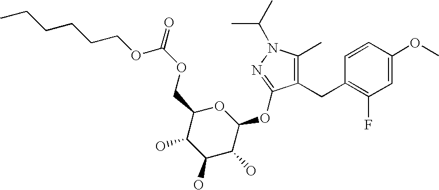

ESI-MS: m/z=569 [M+H]⁺

### (50) 4-(2-Fluoro-4-methoxy-benzyl)-1-isopropyl-5-methyl-3-(6-O-phenoxycarbonyl-β-D-glucopyranos-1-yloxy)-1H-pyrazole

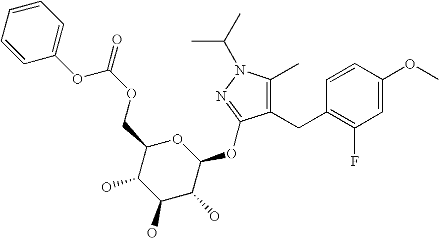

ESI-MS: m/z=561 [M+H]⁺

### (51) 4-(2-Fluoro-4-methoxy-benzyl)-1-isopropyl-5-methyl-3-(6-O-benzyloxycarbonyl-β-D-glucopyranos-1-yloxy)-1H-pyrazole

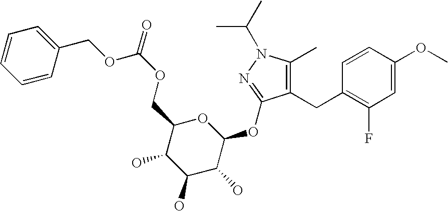

ESI-MS: m/z=575 [M+H]⁺

### (52) 4-(2-Fluoro-4-methoxy-benzyl)-1-isopropyl-5-methyl-3-(6-O-acetyl-β-D-glucopyranos-1-yloxy)-1H-pyrazole

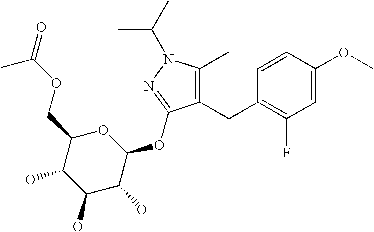

ESI-MS: m/z=483 [M+H]⁺

### (53) 4-(2-Fluoro-4-methoxy-benzyl)-1-isopropyl-5-methyl-3-(6-O-propylcarbonyl-β-D-glucopyranos-1-yloxy)-1H-pyrazole

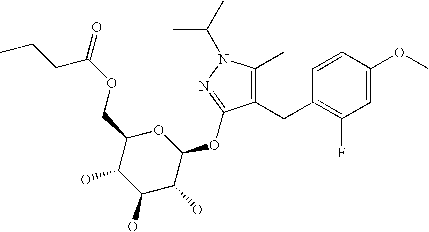

ESI-MS: m/z=511 [M+H]⁺

### (54) 4-(2-Fluoro-4-methoxy-benzyl)-1-isopropyl-5-methyl-3-(6-O-isopropylcarbonyl-β-D-glucopyranos-1-yloxy)-1H-pyrazole

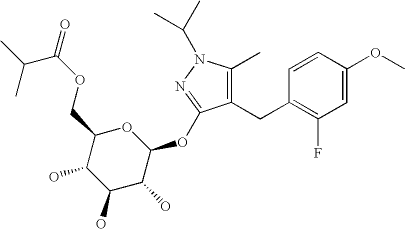

ESI-MS: m/z=511 [M+H]⁺

### (55) 4-(2-Fluoro-4-methoxy-benzyl)-1-isopropyl-5-methyl-3-(6-O-benzylcarbonyl-β-D-glucopyranos-1-yloxy)-1H-pyrazole

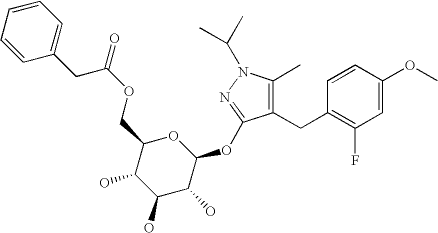

ESI-MS: m/z=559 [M+H]⁺

The compounds (56) to (63) can be obtained by analogy with the procedure described above.

1. A method for preventing, slowing the progression of, delaying or treating a metabolic disorder selected from the group consisting of type 1 diabetes mellitus, type 2 diabetes mellitus, impaired glucose tolerance, hyperglycemia, postprandial hyperglycemia, overweight, obesity and metabolic syndrome, said method comprised of the steps of administering to a patient in need thereof a pharmaceutical composition comprising a pyrazole-O-glucoside derivative selected from the group of compounds (1) to (29) consisting of
(1) 4-(2,3-difluoro-4-methoxy-benzyl)-1-isopropyl-5-methyl-3-β-D-glucopyranos-1-yloxy-1H-pyrazole;
(2) 4-(2,5-difluoro-4-methoxy-benzyl)-1-isopropyl-5-methyl-3-β-D-glucopyranos-1-yloxy-1H-pyrazole;
(3) 4-(2,6-difluoro-4-methoxy-benzyl)-1-isopropyl-5-methyl-3-β-D-glucopyranos-1-yloxy-1H-pyrazole;
(4) 4-(3,5-difluoro-4-methoxy-benzyl)-1-isopropyl-5-methyl-3-β-D-glucopyranos-1-yloxy-1H-pyrazole;
(5) 1-cyclobutyl-4-(3-fluoro-4-methyl-benzyl)-5-methyl-3-β-D-glucopyranos-1-yloxy-1H-pyrazole;
(6) 1-cyclopropylmethyl-4-(3-fluoro-4-methyl-benzyl)-5-methyl-3-β-D-glucopyranos-1-yloxy-1H-pyrazole;
(7) 1-cyclobutyl-4-(2-fluoro-4-methoxy-benzyl)-5-methyl-3-β-D-glucopyranos-1-yloxy-1H-pyrazole;
(8) 4-(3-chloro-4-methoxy-benzyl)-1-isopropyl-5-methyl-3-β-D-glucopyranos-1-yloxy-1H-pyrazole;
(9) 4-(2-chloro-4-methoxy-benzyl)-1-isopropyl-5-methyl-3-β-D-glucopyranos-1-yloxy-1H-pyrazole;
(10) 4-(4-bromo-3-fluoro-benzyl)-1-isopropyl-5-methyl-3-β-D-glucopyranos-1-yloxy-1H-pyrazole;
(11) 4-(2,3-difluoro-4-methyl-benzyl)-1-isopropyl-5-methyl-3-β-D-glucopyranos-1-yloxy-1H-pyrazole;
(12) 4-(2-fluoro-4-methyl-benzyl)-1-isopropyl-5-methyl-3-β-D-glucopyranos-1-yloxy-1H-pyrazole;
(13) 4-(3-fluoro-4-ethoxy-benzyl)-1-isopropyl-5-methyl-3-β-D-glucopyranos-1-yloxy-1H-pyrazole;
(14) 4-(4-ethinyl-benzyl)-1-isopropyl-5-methyl-3-β-D-glucopyranos-1-yloxy-1H-pyrazole;
(15) 4-(3-fluoro-4-isopropoxy-benzyl)-1-isopropyl-5-methyl-3-β-D-glucopyranos-1-yloxy-1H-pyrazole;
(16) 4-(2-fluoro-4-methoxy-benzyl)-1-isopropyl-5-methyl-3-β-D-glucopyranos-1-yloxy-1H-pyrazole;
(17) 4-(2-fluoro-4-methoxy-benzyl)-1-isopropyl-5-trifluoromethyl-3-β-D-glucopyranos-1-yloxy-1H-pyrazole;
(18) 4-(4-bromo-2-fluoro-benzyl)-1-isopropyl-5-methyl-3-β-D-glucopyranos-1-yloxy-1H-pyrazole;
(19) 4-(2-fluoro-4-isopropoxy-benzyl)-1-isopropyl-5-methyl-3-β-D-glucopyranos-1-yloxy-1H-pyrazole;
(20) 4-(2-fluoro-4-ethoxy-benzyl)-1-isopropyl-5-methyl-3-β-D-glucopyranos-1-yloxy-1H-pyrazole;
(21) 4-(4-ethyl-benzyl)-1-isopropyl-5-trifluoromethyl-3-β-D-glucopyranos-1-yloxy-1H-pyrazole;
(22) 4-(4-bromo-benzyl)-1-isopropyl-5-trifluoromethyl-3-β-D-glucopyranos-1-yloxy-1H-pyrazole;
(23) 4-(4-ethyl-benzyl)-1-cyclobutyl-5-trifluoromethyl-3-β-D-glucopyranos-1-yloxy-1H-pyrazole;
(24) 4-(4-ethyl-benzyl)-1-(2-fluoro-1-fluoromethyl-ethyl)-5-trifluoromethyl-3-β-D-glucopyranos-1-yloxy-1H-pyrazole;
(25) 4-(3-fluoro-4-methoxy-benzyl)-1-isopropyl-5-trifluoromethyl-3-β-D-glucopyranos-1-yloxy-1H-pyrazole;
(26) 4-(3-fluoro-4-methyl-benzyl)-1-isopropyl-5-methyl-3-β-D-glucopyranos-1-yloxy-1H-pyrazole;
(27) 4-(2,3-difluoro-4-isopropoxy-benzyl)-1-isopropyl-5-methyl-3-β-D-glucopyranos-1-yloxy-1H-pyrazole;
(28) 4-(3-fluoro-4-methoxy-benzyl)-1-isopropyl-5-methyl-3-β-D-glucopyranos-1-yloxy-1H-pyrazole;
(29) 4-(4-ethyl-benzyl)-1-isopropyl-5-methyl-3-β-D-glucopyranos-1-yloxy-1H-pyrazole;
or a prodrug thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl, phenyl-(C₁₋₃-alkyl)-carbonyl, phenyloxycarbonyl and phenyl-(C₁₋₃-alkyl)-oxycarbonyl, or a pharmaceutically acceptable salt thereof.

2. A method for improving glycemic control and/or for reducing of fasting plasma glucose, of postprandial plasma glucose and/or of glycosylated hemoglobin HbA1c, said method comprised of the steps of administering to a patient in need thereof a a pharmaceutical composition comprising a pyrazole-O-glucoside derivative selected from the group of compounds (1) to (29):
4-(2,3-difluoro-4-methoxy-benzyl)-1-isopropyl-5-methyl-3-β-D-glucopyranos-1-yloxy-1H-pyrazole;
4-(2,5-difluoro-4-methoxy-benzyl)-1-isopropyl-5-methyl-3-β-D-glucopyranos-1-yloxy-1H-pyrazole;
4-(2,6-difluoro-4-methoxy-benzyl)-1-isopropyl-5-methyl-3-β-D-glucopyranos-1-yloxy-1H-pyrazole;
4-(3,5-difluoro-4-methoxy-benzyl)-1-isopropyl-5-methyl-3-β-D-glucopyranos-1-yloxy-1H-pyrazole;
1-cyclobutyl-4-(3-fluoro-4-methyl-benzyl)-5-methyl-3-β-D-glucopyranos-1-yloxy-1H-pyrazole;
1-cyclopropylmethyl-4-(3-fluoro-4-methyl-benzyl)-5-methyl-3-β-D-glucopyranos-1-yloxy-1H-pyrazole;
1-cyclobutyl-4-(2-fluoro-4-methoxy-benzyl)-5-methyl-3-β-D-glucopyranos-1-yloxy-1H-pyrazole;
4-(3-chloro-4-methoxy-benzyl)-1-isopropyl-5-methyl-3-β-D-glucopyranos-1-yloxy-1H-pyrazole;
4-(2-chloro-4-methoxy-benzyl)-1-isopropyl-5-methyl-3-β-D-glucopyranos-1-yloxy-1H-pyrazole;
4-(4-bromo-3-fluoro-benzyl)-1-isopropyl-5-methyl-3-β-D-glucopyranos-1-yloxy-1H-pyrazole;
4-(2,3-difluoro-4-methyl-benzyl)-1-isopropyl-5-methyl-3-β-D-glucopyranos-1-yloxy-1H-pyrazole;
4-(2-fluoro-4-methyl-benzyl)-1-isopropyl-5-methyl-3-β-D-glucopyranos-1-yloxy-1H-pyrazole;
4-(3-fluoro-4-ethoxy-benzyl)-1-isopropyl-5-methyl-3-β-D-glucopyranos-1-yloxy-1H-pyrazole;
4-(4-ethinyl-benzyl)-1-isopropyl-5-methyl-3-β-D-glucopyranos-1-yloxy-1H-pyrazole;
4-(3-fluoro-4-isopropoxy-benzyl)-1-isopropyl-5-methyl-3-β-D-glucopyranos-1-yloxy-1H-pyrazole;
4-(2-fluoro-4-methoxy-benzyl)-1-isopropyl-5-methyl-3-β-D-glucopyranos-1-yloxy-1H-pyrazole;
4-(2-fluoro-4-methoxy-benzyl)-1-isopropyl-5-trifluoromethyl-3-β-D-glucopyranos-1-yloxy-1H-pyrazole;
4-(4-bromo-2-fluoro-benzyl)-1-isopropyl-5-methyl-3-β-D-glucopyranos-1-yloxy-1H-pyrazole;
4-(2-fluoro-4-isopropoxy-benzyl)-1-isopropyl-5-methyl-3-β-D-glucopyranos-1-yloxy-1H-pyrazole;
4-(2-fluoro-4-ethoxy-benzyl)-1-isopropyl-5-methyl-3-β-D-glucopyranos-1-yloxy-1H-pyrazole;
4-(4-ethyl-benzyl)-1-isopropyl-5-trifluoromethyl-3-β-D-glucopyranos-1-yloxy-1H-pyrazole;
4-(4-bromo-benzyl)-1-isopropyl-5-trifluoromethyl-3-β-D-glucopyranos-1-yloxy-1H-pyrazole;
4-(4-ethyl-benzyl)-1-cyclobutyl-5-trifluoromethyl-3-β-D-glucopyranos-1-yloxy-1H-pyrazole;
4-(4-ethyl-benzyl)-1-(2-fluoro-1-fluoromethyl-ethyl)-5-trifluoromethyl-3-β-D-glucopyranos-1-yloxy-1H-pyrazole;
4-(3-fluoro-4-methoxy-benzyl)-1-isopropyl-5-trifluoromethyl-3-β-D-glucopyranos-1-yloxy-1H-pyrazole;
4-(3-fluoro-4-methyl-benzyl)-1-isopropyl-5-methyl-3-β-D-glucopyranos-1-yloxy-1H-pyrazole;
4-(2,3-difluoro-4-isopropoxy-benzyl)-1-isopropyl-5-methyl-3-β-D-glucopyranos-1-yloxy-1H-pyrazole;
4-(3-fluoro-4-methoxy-benzyl)-1-isopropyl-5-methyl-3-β-D-glucopyranos-1-yloxy-1H-pyrazole;
4-(4-ethyl-benzyl)-1-isopropyl-5-methyl-3-β-D-glucopyranos-1-yloxy-1H-pyrazole;
or a prodrug thereof, or a pharmaceutically acceptable salt thereof, defined as in claim 1.

3. A method for preventing, slowing, delaying or reversing progression from impaired glucose tolerance, insulin resistance and/or from metabolic syndrome to type 2 diabetes mellitus to a patient in need thereof a pharmaceutical composition comprising a pyrazole-O-glucoside derivative selected from the group of compounds (1) to (29):
4-(2,5-difluoro-4-methoxy-benzyl)-1-isopropyl-5-methyl-3-β-D-glucopyranos-1-yloxy-1H-pyrazole;
4-(2,6-difluoro-4-methoxy-benzyl)-1-isopropyl-5-methyl-3-β-D-glucopyranos-1-yloxy-1H-pyrazole;
4-(3,5-difluoro-4-methoxy-benzyl)-1-isopropyl-5-methyl-3-β-D-glucopyranos-1-yloxy-1H-pyrazole;
1-cyclobutyl-4-(3-fluoro-4-methyl-benzyl)-5-methyl-3-β-D-glucopyranos-1-yloxy-1H-pyrazole;
1-cyclopropylmethyl-4-(3-fluoro-4-methyl-benzyl)-5-methyl-3-β-D-glucopyranos-1-yloxy-1H-pyrazole;
1-cyclobutyl-4-(2-fluoro-4-methoxy-benzyl)-5-methyl-3-β-D-glucopyranos-1-yloxy-1H-pyrazole;
4-(3-chloro-4-methoxy-benzyl)-1-isopropyl-5-methyl-3-β-D-glucopyranos-1-yloxy-1H-pyrazole;
4-(2-chloro-4-methoxy-benzyl)-1-isopropyl-5-methyl-3-β-D-glucopyranos-1-yloxy-1H-pyrazole;
4-(4-bromo-3-fluoro-benzyl)-1-isopropyl-5-methyl-3-β-D-glucopyranos-1-yloxy-1H-pyrazole;
4-(2,3-difluoro-4-methyl-benzyl)-1-isopropyl-5-methyl-3-β-D-glucopyranos-1-yloxy-1H-pyrazole;
4-(2-fluoro-4-methyl-benzyl)-1-isopropyl-5-methyl-3-β-D-glucopyranos-1-yloxy-1H-pyrazole;
4-(3-fluoro-4-ethoxy-benzyl)-1-isopropyl-5-methyl-3-β-D-glucopyranos-1-yloxy-1H-pyrazole;
4-(4-ethinyl-benzyl)-1-isopropyl-5-methyl-3-β-D-glucopyranos-1-yloxy-1H-pyrazole;
4-(3-fluoro-4-isopropoxy-benzyl)-1-isopropyl-5-methyl-3-β-D-glucopyranos-1-yloxy-1H-pyrazole;
4-(2-fluoro-4-methoxy-benzyl)-1-isopropyl-5-methyl-3-β-D-glucopyranos-1-yloxy-1H-pyrazole;
4-(2-fluoro-4-methoxy-benzyl)-1-isopropyl-5-trifluoromethyl-3-β-D-glucopyranos-1-yloxy-1H-pyrazole;
4-(4-bromo-2-fluoro-benzyl)-1-isopropyl-5-methyl-3-β-D-glucopyranos-1-yloxy-1H-pyrazole;
4-(2-fluoro-4-isopropoxy-benzyl)-1-isopropyl-5-methyl-3-β-D-glucopyranos-1-yloxy-1H-pyrazole;
4-(2-fluoro-4-ethoxy-benzyl)-1-isopropyl-5-methyl-3-β-D-glucopyranos-1-yloxy-1H-pyrazole;
4-(4-ethyl-benzyl)-1-isopropyl-5-trifluoromethyl-3-β-D-glucopyranos-1-yloxy-1H-pyrazole;
4-(4-bromo-benzyl)-1-isopropyl-5-trifluoromethyl-3-β-D-glucopyranos-1-yloxy-1H-pyrazole;
4-(4-ethyl-benzyl)-1-cyclobutyl-5-trifluoromethyl-3-β-D-glucopyranos-1-yloxy-1H-pyrazole;
4-(4-ethyl-benzyl)-1-(2-fluoro-1-fluoromethyl-ethyl)-5-trifluoromethyl-3-β-D-glucopyranos-1-yloxy-1H-pyrazole;
4-(3-fluoro-4-methoxy-benzyl)-1-isopropyl-5-trifluoromethyl-3-β-D-glucopyranos-1-yloxy-1H-pyrazole;
4-(3-fluoro-4-methyl-benzyl)-1-isopropyl-5-methyl-3-β-D-glucopyranos-1-yloxy-1H-pyrazole;
4-(2,3-difluoro-4-isopropoxy-benzyl)-1-isopropyl-5-methyl-3-β-D-glucopyranos-1-yloxy-1H-pyrazole;
4-(3-fluoro-4-methoxy-benzyl)-1-isopropyl-5-methyl-3-β-D-glucopyranos-1-yloxy-1H-pyrazole;
4-(4-ethyl-benzyl)-1-isopropyl-5-methyl-3-β-D-glucopyranos-1-yloxy-1H-pyrazole;
or a prodrug thereof, or a pharmaceutically acceptable salt thereof as defined in claim 1.

4. A method for preventing, slowing the progression of, delaying or treating of a condition or disorder selected from the group consisting of complications of diabetes mellitus such as cataracts and micro- and macrovascular diseases, such as nephropathy, retinopathy, neuropathy, tissue ischaemia, arteriosclerosis, myocardial infarction, stroke and peripheral arterial occlusive disease, said method comprised of the steps of administering to a patient in need thereof a pharmaceutical composition comprising a pyrazole-O-glucoside derivative selected from the group of compounds (1) to (29),
4-(2,3-difluoro-4-methoxy-benzyl)-1-isopropyl-5-methyl-3-β-D-glucopyranos-1-yloxy-1H-pyrazole;
4-(2,5-difluoro-4-methoxy-benzyl)-1-isopropyl-5-methyl-3-β-D-glucopyranos-1-yloxy-1H-pyrazole;
4-(2,6-difluoro-4-methoxy-benzyl)-1-isopropyl-5-methyl-3-β-D-glucopyranos-1-yloxy-1H-pyrazole;
4-(3,5-difluoro-4-methoxy-benzyl)-1-isopropyl-5-methyl-3-β-D-glucopyranos-1-yloxy-1H-pyrazole;
1-cyclobutyl-4-(3-fluoro-4-methyl-benzyl)-5-methyl-3-β-D-glucopyranos-1-yloxy-1H-pyrazole;
1-cyclopropylmethyl-4-(3-fluoro-4-methyl-benzyl)-5-methyl-3-β-D-glucopyranos-1-yloxy-1H-pyrazole;
1-cyclobutyl-4-(2-fluoro-4-methoxy-benzyl)-5-methyl-3-β-D-glucopyranos-1-yloxy-1H-pyrazole;
4-(3-chloro-4-methoxy-benzyl)-1-isopropyl-5-methyl-3-β-D-glucopyranos-1-yloxy-1H-pyrazole;
4-(2-chloro-4-methoxy-benzyl)-1-isopropyl-5-methyl-3-β-D-glucopyranos-1-yloxy-1H-pyrazole;
4-(4-bromo-3-fluoro-benzyl)-1-isopropyl-5-methyl-3-β-D-glucopyranos-1-yloxy-1H-pyrazole;
4-(2,3-difluoro-4-methyl-benzyl)-1-isopropyl-5-methyl-3-β-D-glucopyranos-1-yloxy-1H-pyrazole;
4-(2-fluoro-4-methyl-benzyl)-1-isopropyl-5-methyl-3-β-D-glucopyranos-1-yloxy-1H-pyrazole;
4-(3-fluoro-4-ethoxy-benzyl)-1-isopropyl-5-methyl-3-β-D-glucopyranos-1-yloxy-1H-pyrazole;
4-(4-ethinyl-benzyl)-1-isopropyl-5-methyl-3-β-D-glucopyranos-1-yloxy-1H-pyrazole;
4-(3-fluoro-4-isopropoxy-benzyl)-1-isopropyl-5-methyl-3-β-D-glucopyranos-1-yloxy-1H-pyrazole;
4-(2-fluoro-4-methoxy-benzyl)-1-isopropyl-5-methyl-3-β-D-glucopyranos-1-yloxy-1H-pyrazole;
4-(2-fluoro-4-methoxy-benzyl)-1-isopropyl-5-trifluoromethyl-3-β-D-glucopyranos-1-yloxy-1H-pyrazole;
4-(4-bromo-2-fluoro-benzyl)-1-isopropyl-5-methyl-3-β-D-glucopyranos-1-yloxy-1H-pyrazole;
4-(2-fluoro-4-isopropoxy-benzyl)-1-isopropyl-5-methyl-3-β-D-glucopyranos-1-yloxy-1H-pyrazole;
4-(2-fluoro-4-ethoxy-benzyl)-1-isopropyl-5-methyl-3-β-D-glucopyranos-1-yloxy-1H-pyrazole;
4-(4-ethyl-benzyl)-1-isopropyl-5-trifluoromethyl-3-β-D-glucopyranos-1-yloxy-1H-pyrazole;
4-(4-bromo-benzyl)-1-isopropyl-5-trifluoromethyl-3-β-D-glucopyranos-1-yloxy-1H-pyrazole;
4-(4-ethyl-benzyl)-1-cyclobutyl-5-trifluoromethyl-3-β-D-glucopyranos-1-yloxy-1H-pyrazole;
4-(4-ethyl-benzyl)-1-(2-fluoro-1-fluoromethyl-ethyl)-5-trifluoromethyl-3-β-D-glucopyranos-1-yloxy-1H-pyrazole;
4-(3-fluoro-4-methoxy-benzyl)-1-isopropyl-5-trifluoromethyl-3-β-D-glucopyranos-1-yloxy-1H-pyrazole;
4-(3-fluoro-4-methyl-benzyl)-1-isopropyl-5-methyl-3-β-D-glucopyranos-1-yloxy-1H-pyrazole;
4-(2,3-difluoro-4-isopropoxy-benzyl)-1-isopropyl-5-methyl-3-β-D-glucopyranos-1-yloxy-1H-pyrazole;
4-(3-fluoro-4-methoxy-benzyl)-1-isopropyl-5-methyl-3-β-D-glucopyranos-1-yloxy-1H-pyrazole;
4-(4-ethyl-benzyl)-1-isopropyl-5-methyl-3-β-D-glucopyranos-1-yloxy-1H-pyrazole;
or a prodrug thereof, or a pharmaceutically acceptable salt thereof.

5. A method for reducing the weight or preventing an increase of the weight or facilitating a reduction of the weight said method comprising the steps of administering to a patient in need thereof a pharmaceutical composition comprising a pyrazole-O-glucoside derivative selected from the group of compounds (1) to (29):
4-(2,3-difluoro-4-methoxy-benzyl)-1-isopropyl-5-methyl-3-β-D-glucopyranos-1-yloxy-1H-pyrazole;
4-(2,5-difluoro-4-methoxy-benzyl)-1-isopropyl-5-methyl-3-β-D-glucopyranos-1-yloxy-1H-pyrazole;
4-(2,6-difluoro-4-methoxy-benzyl)-1-isopropyl-5-methyl-3-β-D-glucopyranos-1-yloxy-1H-pyrazole;
4-(3,5-difluoro-4-methoxy-benzyl)-1-isopropyl-5-methyl-3-β-D-glucopyranos-1-yloxy-1H-pyrazole;
1-cyclobutyl-4-(3-fluoro-4-methyl-benzyl)-5-methyl-3-β-D-glucopyranos-1-yloxy-1H-pyrazole;
1-cyclopropylmethyl-4-(3-fluoro-4-methyl-benzyl)-5-methyl-3-β-D-glucopyranos-1-yloxy-1H-pyrazole;
1-cyclobutyl-4-(2-fluoro-4-methoxy-benzyl)-5-methyl-3-β-D-glucopyranos-1-yloxy-1H-pyrazole;
4-(3-chloro-4-methoxy-benzyl)-1-isopropyl-5-methyl-3-β-D-glucopyranos-1-yloxy-1H-pyrazole;
4-(2-chloro-4-methoxy-benzyl)-1-isopropyl-5-methyl-3-β-D-glucopyranos-1-yloxy-1H-pyrazole;
4-(4-bromo-3-fluoro-benzyl)-1-isopropyl-5-methyl-3-β-D-glucopyranos-1-yloxy-1H-pyrazole;
4-(2,3-difluoro-4-methyl-benzyl)-1-isopropyl-5-methyl-3-β-D-glucopyranos-1-yloxy-1H-pyrazole;
4-(2-fluoro-4-methyl-benzyl)-1-isopropyl-5-methyl-3-β-D-glucopyranos-1-yloxy-1H-pyrazole;
4-(3-fluoro-4-ethoxy-benzyl)-1-isopropyl-5-methyl-3-β-D-glucopyranos-1-yloxy-1H-pyrazole;
4-(4-ethinyl-benzyl)-1-isopropyl-5-methyl-3-β-D-glucopyranos-1-yloxy-1H-pyrazole;
4-(3-fluoro-4-isopropoxy-benzyl)-1-isopropyl-5-methyl-3-β-D-glucopyranos-1-yloxy-1H-pyrazole;
4-(2-fluoro-4-methoxy-benzyl)-1-isopropyl-5-methyl-3-β-D-glucopyranos-1-yloxy-1H-pyrazole;
4-(2-fluoro-4-methoxy-benzyl)-1-isopropyl-5-trifluoromethyl-3-β-D-glucopyranos-1-yloxy-1H-pyrazole;
4-(4-bromo-2-fluoro-benzyl)-1-isopropyl-5-methyl-3-β-D-glucopyranos-1-yloxy-1H-pyrazole;
4-(2-fluoro-4-isopropoxy-benzyl)-1-isopropyl-5-methyl-3-β-D-glucopyranos-1-yloxy-1H-pyrazole;
4-(2-fluoro-4-ethoxy-benzyl)-1-isopropyl-5-methyl-3-β-D-glucopyranos-1-yloxy-1H-pyrazole;
4-(4-ethyl-benzyl)-1-isopropyl-5-trifluoromethyl-3-β-D-glucopyranos-1-yloxy-1H-pyrazole;
4-(4-bromo-benzyl)-1-isopropyl-5-trifluoromethyl-3-β-D-glucopyranos-1-yloxy-1H-pyrazole;
4-(4-ethyl-benzyl)-1-cyclobutyl-5-trifluoromethyl-3-β-D-glucopyranos-1-yloxy-1H-pyrazole;
4-(4-ethyl-benzyl)-1-(2-fluoro-1-fluoromethyl-ethyl)-5-trifluoromethyl-3-β-D-glucopyranos-1-yloxy-1H-pyrazole;
4-(3-fluoro-4-methoxy-benzyl)-1-isopropyl-5-trifluoromethyl-3-β-D-glucopyranos-1-yloxy-1H-pyrazole;
4-(3-fluoro-4-methyl-benzyl)-1-isopropyl-5-methyl-3-β-D-glucopyranos-1-yloxy-1H-pyrazole;
4-(2,3-difluoro-4-isopropoxy-benzyl)-1-isopropyl-5-methyl-3-β-D-glucopyranos-1-yloxy-1H-pyrazole;
4-(3-fluoro-4-methoxy-benzyl)-1-isopropyl-5-methyl-3-β-D-glucopyranos-1-yloxy-1H-pyrazole;
4-(4-ethyl-benzyl)-1-isopropyl-5-methyl-3-β-D-glucopyranos-1-yloxy-1H-pyrazole;
or a prodrug thereof, or a pharmaceutically acceptable salt thereof as defined as in claim 1.

6. A method for preventing, slowing, delaying or treating the degeneration of pancreatic beta cells and/or the decline of the functionality of pancreatic beta cells and/or for improving and/or restoring the functionality of pancreatic beta cells and/or restoring the functionality of pancreatic insulin secretion in a patient in need thereof, said method comprised of the steps of administering to a patient in need thereof a pharmaceutical composition comprising a pyrazole-O-glucoside derivative selected from the group of compounds (1) to (29),
2,3-difluoro-4-methoxy-benzyl)-1-isopropyl-5-methyl-3-β-D-glucopyranos-1-yloxy-1H-pyrazole;
4-(2,5-difluoro-4-methoxy-benzyl)-1-isopropyl-5-methyl-3-β-D-glucopyranos-1-yloxy-1H-pyrazole;
4-(2,6-difluoro-4-methoxy-benzyl)-1-isopropyl-5-methyl-3-β-D-glucopyranos-1-yloxy-1H-pyrazole;
4-(3,5-difluoro-4-methoxy-benzyl)-1-isopropyl-5-methyl-3-β-D-glucopyranos-1-yloxy-1H-pyrazole;
1-cyclobutyl-4-(3-fluoro-4-methyl-benzyl)-5-methyl-3-β-D-glucopyranos-1-yloxy-1H-pyrazole;
1-cyclopropymethyl-4-(3-fluoro-4-methyl-benzyl)-5-methyl-3-β-D-glucopyranos-1-yloxy-1H-pyrazole;
1-cyclobutyl-4-(2-fluoro-4-methoxy-benzyl)-5-methyl-3-β-D-glucopyranos-1-yloxy-1H-pyrazole;
4-(3-chloro-4-methoxy-benzyl)-1-isopropyl-5-methyl-3-β-D-glucopyranos-1-yloxy-1H-pyrazole;
4-(2-chloro-4-methoxy-benzyl)-1-isopropyl-5-methyl-3-β-D-glucopyranos-1-yloxy-1H-pyrazole;
4-(4-bromo-3-fluoro-benzyl)-1-isopropyl-5-methyl-3-β-D-glucopyranos-1-yloxy-1H-pyrazole;
4-(2,3-difluoro-4-methyl-benzyl)-1-isopropyl-5-methyl-3-β-D-glucopyranos-1-yloxy-1H-pyrazole;
4-(2-fluoro-4-methyl-benzyl)-1-isopropyl-5-methyl-3-β-D-glucopyranos-1-yloxy-1H-pyrazole;
4-(3-fluoro-4-ethoxy-benzyl)-1-isopropyl-5-methyl-3-β-D-glucopyranos-1-yloxy-1H-pyrazole;
4-(4-ethinyl-benzyl)-1-isopropyl-5-methyl-3-β-D-glucopyranos-1-yloxy-1H-pyrazole;
4-(3-fluoro-4-isopropoxy-benzyl)-1-isopropyl-5-methyl-3-β-D-glucopyranos-1-yloxy-1H-pyrazole;
4-(2-fluoro-4-methoxy-benzyl)-1-isopropyl-5-methyl-3-β-D-glucopyranos-1-yloxy-1H-pyrazole;
4-(2-fluoro-4-methoxy-benzyl)-1-isopropyl-5-trifluoromethyl-3-β-D-glucopyranos-1-yloxy-1H-pyrazole;
4-(4-bromo-2-fluoro-benzyl)-1-isopropyl-5-methyl-3-β-D-glucopyranos-1-yloxy-1H-pyrazole;
4-(2-fluoro-4-isopropoxy-benzyl)-1-isopropyl-5-methyl-3-β-D-glucopyranos-1-yloxy-1H-pyrazole;
4-(2-fluoro-4-ethoxy-benzyl)-1-isopropyl-5-methyl-3-β-D-glucopyranos-1-yloxy-1H-pyrazole;
4-(4-ethyl-benzyl)-1-isopropyl-5-trifluoromethyl-3-β-D-glucopyranos-1-yloxy-1H-pyrazole;
4-(4-bromo-benzyl)-1-isopropyl-5-trifluoromethyl-3-β-D-glucopyranos-1-yloxy-1H-pyrazole;
4-(4-ethyl-benzyl)-1-cyclobutyl-5-trifluoromethyl-3-β-D-glucopyranos-1-yloxy-1H-pyrazole;
4-(4-ethyl-benzyl)-1-(2-fluoro-1-fluoromethyl-ethyl)-5-trifluoromethyl-3-β-D-glucopyranos-1-yloxy-1H-pyrazole;
4-(3-fluoro-4-methoxy-benzyl)-1-isopropyl-5-trifluoromethyl-3-β-D-glucopyranos-1-yloxy-1H-pyrazole;
4-(3-fluoro-4-methyl-benzyl)-1-isopropyl-5-methyl-3-β-D-glucopyranos-1-yloxy-1H-pyrazole;
4-(2,3-difluoro-4-isopropoxy-benzyl)-1-isopropyl-5-methyl-3-β-D-glucopyranos-1-yloxy-1H-pyrazole;
4-(3-fluoro-4-methoxy-benzyl)-1-isopropyl-5-methyl-3-β-D-glucopyranos-1-yloxy-1H-pyrazole;
4-(4-ethyl-benzyl)-1-isopropyl-5-methyl-3-β-D-glucopyranos-1-yloxy-1H-pyrazole;
or a prodrug thereof, or a pharmaceutically acceptable salt thereof.

7. A method for maintaining and/or improving the insulin sensitivity and/or for treating or preventing hyperinsulinemia and/or insulin resistance in a patient, said method comprised of the steps of administering to a patient in need thereof a pharmaceutical composition comprising a pyrazole-O-glucoside derivative selected from the group of compounds (1) to (29):
3-difluoro-4-methoxy-benzyl)-1-isopropyl-5-methyl-3-β-D-glucopyranos-1-yloxy-1H-pyrazole;
5-difluoro-4-methoxy-benzyl)-1-isopropyl-5-methyl-3-β-D-glucopyranos-1-yloxy-1H-pyrazole;
6-difluoro-4-methoxy-benzyl)-1-isopropyl-5-methyl-3-β-D-glucopyranos-1-yloxy-1H-pyrazole;
5-difluoro-4-methoxy-benzyl)-1-isopropyl-5-methyl-3-β-D-glucopyranos-1-yloxy-1H-pyrazole;
clobutyl-4-(3-fluoro-4-methyl-benzyl)-5-methyl-3-β-D-glucopyranos-1-yloxy-1H-pyrazole;
clopropylmethyl-4-(3-fluoro-4-methyl-benzyl)-5-methyl-3-β-D-glucopyranos-1-yloxy-1H-pyrazole;
clobutyl-4-(2-fluoro-4-methoxy-benzyl)-5-methyl-3-β-D-glucopyranos-1-yloxy-1H-pyrazole;
chloro-4-methoxy-benzyl)-1-isopropyl-5-methyl-3-β-D-glucopyranos-1-yloxy-1H-pyrazole;
chloro-4-methoxy-benzyl)-1-isopropyl-5-methyl-3-β-D-glucopyranos-1-yloxy-1H-pyrazole;
bromo-3-fluoro-benzyl)-1-isopropyl-5-methyl-3-β-D-glucopyranos-1-yloxy-1H-pyrazole;
3-difluoro-4-methyl-benzyl)-1-isopropyl-5-methyl-3-β-D-glucopyranos-1-yloxy-1H-pyrazole;
fluoro-4-methyl-benzyl)-1-isopropyl-5-methyl-3-β-D-glucopyranos-1-yloxy-1H-pyrazole;
fluoro-4-ethoxy-benzyl)-1-isopropyl-5-methyl-3-β-D-glucopyranos-1-yloxy-1H-pyrazole;
ethinyl-benzyl)-1-isopropyl-5-methyl-3-β-D-glucopyranos-1-yloxy-1H-pyrazole;
fluoro-4-isopropoxy-benzyl)-1-isopropyl-5-methyl-3-β-D-glucopyranos-1-yloxy-1H-pyrazole;
fluoro-4-methoxy-benzyl)-1-isopropyl-5-methyl-3-β-D-glucopyranos-1-yloxy-1H-pyrazole;
fluoro-4-methoxy-benzyl)-1-isopropyl-5-trifluoromethyl-3-β-D-glucopyranos-1-yloxy-1H-pyrazole;
bromo-2-fluoro-benzyl)-1-isopropyl-5-methyl-3-β-D-glucopyranos-1-yloxy-1H-pyrazole;
fluoro-4-isopropoxy-benzyl)-1-isopropyl-5-methyl-3-β-D-glucopyranos-1-yloxy-1H-pyrazole;
fluoro-4-ethoxy-benzyl)-1-isopropyl-5-methyl-3-β-D-glucopyranos-1-yloxy-1H-pyrazole;
ethyl-benzyl)-1-isopropyl-5-trifluoromethyl-3-β-D-glucopyranos-1-yloxy-1H-pyrazole;
bromo-benzyl)-1-isopropyl-5-trifluoromethyl-3-β-D-glucopyranos-1-yloxy-1H-pyrazole;
ethyl-benzyl)-1-cyclobutyl-5-trifluoromethyl-3-β-D-glucopyranos-1-yloxy-1H-pyrazole;
ethyl-benzyl)-1-(2-fluoro-1-fluoromethyl-ethyl)-5-trifluoromethyl-3-β-D-glucopyranos-1-yloxy-1H-pyrazole;
fluoro-4-methoxy-benzyl)-1-isopropyl-5-trifluoromethyl-3-β-D-glucopyranos-1-yloxy-1H-pyrazole;
fluoro-4-methyl-benzyl)-1-isopropyl-5-methyl-3-β-D-glucopyranos-1-yloxy-1H-pyrazole;
3-difluoro-4-isopropoxy-benzyl)-1-isopropyl-5-methyl-3-β-D-glucopyranos-1-yloxy-1H-pyrazole;
fluoro-4-methoxy-benzyl)-1-isopropyl-5-methyl-3-β-D-glucopyranos-1-yloxy-1H-pyrazole;
ethyl-benzyl)-1-isopropyl-5-methyl-3-β-D-glucopyranos-1-yloxy-1H-pyrazole;
or a prodrug thereof, or a pharmaceutically acceptable salt thereof, defined as in claim 1 is administered.

8. A method according to claim 1 wherein the said pharmaceutical composition is comprised of 1 mg to 1000 mg of said pyrazole-O-glucoside derivative

9. A method according to one claim 1 wherein said patient is an individual diagnosed of one or more of the conditions selected from the group consisting of overweight, obesity, visceral obesity and abdominal obesity.

10. A method according to claim 1 wherein the patient is an individual who shows one, two or more of the following conditions:
(a) a fasting blood glucose or serum glucose concentration greater than 110 mg/dL, in particular greater than 125 mg/dL;
(b) a postprandial plasma glucose equal to or greater than 140 mg/dL;
(c) an HbA1c value equal to or greater than 6.5%, in particular equal to or greater than 8.0%.

11. A method according to claim 1 wherein the patient is an individual wherein one, two, three or more of the following conditions are present:
(a) obesity, visceral obesity and/or abdominal obesity,
(b) triglyceride blood level ≧150 mg/dL,
(c) HDL-cholesterol blood level <40 mg/dL in female patients and <50 mg/dL in male patients,
(d) a systolic blood pressure ≧130 mm Hg and a diastolic blood pressure ≧85 mm Hg,
(e) a fasting blood glucose level ≧110 mg/dL.

12. A method according to claim 1 wherein the patient is an individual for whom the treatment with metformin is contraindicated and/or who has an intolerance against metformin at therapeutic doses.

13. A method according to claim 1 wherein the patient is an individual with insufficient glycemic control despite treatment with one or more antidiabetic drugs.

14. A method according to claim 1 wherein the pyrazole-O-glucoside derivative is a prodrug selected from the group of compounds (1) to (29)
4-(2,3-difluoro-4-methoxy-benzyl)-1-isopropyl-5-methyl-3-β-D-glucopyranos-1-yloxy-1H-pyrazole;
4-(2,5-difluoro-4-methoxy-benzyl)-1-isopropyl-5-methyl-3-β-D-glucopyranos-1-yloxy-1H-pyrazole;
4-(2,6-difluoro-4-methoxy-benzyl)-1-isopropyl-5-methyl-3-β-D-glucopyranos-1-yloxy-1H-pyrazole;
4-(3,5-difluoro-4-methoxy-benzyl)-1-isopropyl-5-methyl-3-β-D-glucopyranos-1-yloxy-1H-pyrazole;
1-cyclobutyl-4-(3-fluoro-4-methyl-benzyl)-5-methyl-3-β-D-glucopyranos-1-yloxy-1H-pyrazole;
1-cyclopropylmethyl-4-(3-fluoro-4-methyl-benzyl)-5-methyl-3-β-D-glucopyranos-1-yloxy-1H-pyrazole;
1-cyclobutyl-4-(2-fluoro-4-methoxy-benzyl)-5-methyl-3-β-D-glucopyranos-1-yloxy-1H-pyrazole;
4-(3-chloro-4-methoxy-benzyl)-1-isopropyl-5-methyl-3-β-D-glucopyranos-1-yloxy-1H-pyrazole;
4-(2-chloro-4-methoxy-benzyl)-1-isopropyl-5-methyl-3-β-D-glucopyranos-1-yloxy-1H-pyrazole;
4-(4-bromo-3-fluoro-benzyl)-1-isopropyl-5-methyl-3-β-D-glucopyranos-1-yloxy-1H-pyrazole;
4-(2,3-difluoro-4-methyl-benzyl)-1-isopropyl-5-methyl-3-β-D-glucopyranos-1-yloxy-1H-pyrazole;
4-(2-fluoro-4-methyl-benzyl)-1-isopropyl-5-methyl-3-β-D-glucopyranos-1-yloxy-1H-pyrazole;
4-(3-fluoro-4-ethoxy-benzyl)-1-isopropyl-5-methyl-3-β-D-glucopyranos-1-yloxy-1H-pyrazole;
4-(4-ethinyl-benzyl)-1-isopropyl-5-methyl-3-β-D-glucopyranos-1-yloxy-1H-pyrazole;
4-(3-fluoro-4-isopropoxy-benzyl)-1-isopropyl-5-methyl-3-β-D-glucopyranos-1-yloxy-1H-pyrazole;
4-(2-fluoro-4-methoxy-benzyl)-1-isopropyl-5-methyl-3-β-D-glucopyranos-1-yloxy-1H-pyrazole;
4-(2-fluoro-4-methoxy-benzyl)-1-isopropyl-5-trifluoromethyl-3-β-D-glucopyranos-1-yloxy-1H-pyrazole;
4-(4-bromo-2-fluoro-benzyl)-1-isopropyl-5-methyl-3-β-D-glucopyranos-1-yloxy-1H-pyrazole;
4-(2-fluoro-4-isopropoxy-benzyl)-1-isopropyl-5-methyl-3-β-D-glucopyranos-1-yloxy-1H-pyrazole;
4-(2-fluoro-4-ethoxy-benzyl)-1-isopropyl-5-methyl-3-β-D-glucopyranos-1-yloxy-1H-pyrazole;
4-(4-ethyl-benzyl)-1-isopropyl-5-trifluoromethyl-3-β-D-glucopyranos-1-yloxy-1H-pyrazole;
4-(4-bromo-benzyl)-1-isopropyl-5-trifluoromethyl-3-β-D-glucopyranos-1-yloxy-1H-pyrazole;
4-(4-ethyl-benzyl)-1-cyclobutyl-5-trifluoromethyl-3-β-D-glucopyranos-1-yloxy-1H-pyrazole;
4-(4-ethyl-benzyl)-1-(2-fluoro-1-fluoromethyl-ethyl)-5-trifluoromethyl-3-β-D-glucopyranos-1-yloxy-1H-pyrazole;
4-(3-fluoro-4-methoxy-benzyl)-1-isopropyl-5-trifluoromethyl-3-β-D-glucopyranos-1-yloxy-1H-pyrazole;
4-(3-fluoro-4-methyl-benzyl)-1-isopropyl-5-methyl-3-β-D-glucopyranos-1-yloxy-1H-pyrazole;
4-(2,3-difluoro-4-isopropoxy-benzyl)-1-isopropyl-5-methyl-3-β-D-glucopyranos-1-yloxy-1H-pyrazole;
4-(3-fluoro-4-methoxy-benzyl)-1-isopropyl-5-methyl-3-β-D-glucopyranos-1-yloxy-1H-pyrazole;
4-(4-ethyl-benzyl)-1-isopropyl-5-methyl-3-β-D-glucopyranos-1-yloxy-1H-pyrazole as defined as in claim 1, wherein the hydrogen atom of the hydroxyl group in 6-position of the β-D-glucopyranosyl-group is replaced by a group selected from among (C₁₋₃-alkyl)carbonyl, (C₁₋₆-alkyl)oxycarbonyl, phenyloxycarbonyl, benzyloxycarbonyl and benzylcarbonyl, or a pharmaceutically acceptable salt thereof.

15. A method according to claim 14 wherein the prodrug of a pyrazole-O-glucoside derivative is selected from the group of compounds selected from,
(30a) 4-(2,3-difluoro-4-methoxy-benzyl)-1-isopropyl-5-methyl-3-(6-O-methoxycarbonyl-β-D-glucopyranos-1-yloxy)-1H-pyrazole;
(30b) 4-(2,3-difluoro-4-methoxy-benzyl)-1-isopropyl-5-methyl-3-(6-O-ethoxycarbonyl-β-D-glucopyranos-1-yloxy)-1H-pyrazole;
(31a) 4-(3-fluoro-4-ethoxy-benzyl)-1-isopropyl-5-methyl-3-(6-O-methoxycarbonyl-β-D-glucopyranos-1-yloxy)-1H-pyrazole;
(31b) 4-(3-fluoro-4-ethoxy-benzyl)-1-isopropyl-5-methyl-3-(6-O-ethoxycarbonyl-β-D-glucopyranos-1-yloxy)-1H-pyrazole;
(32a) 4-(3-fluoro-4-isopropoxy-benzyl)-1-isopropyl-5-methyl-3-(6-O-methoxycarbonyl-β-D-glucopyranos-1-yloxy)-1H-pyrazole;
(32b) 4-(3-fluoro-4-isopropoxy-benzyl)-1-isopropyl-5-methyl-3-(6-O-ethoxycarbonyl-β-D-glucopyranos-1-yloxy)-1H-pyrazole;
(33a) 4-(2,5-difluoro-4-methoxy-benzyl)-1-isopropyl-5-methyl-3-(6-O-methoxycarbonyl-β-D-glucopyranos-1-yloxy)-1H-pyrazole;
(33b) 4-(2,5-difluoro-4-methoxy-benzyl)-1-isopropyl-5-methyl-3-(6-O-ethoxycarbonyl-β-D-glucopyranos-1-yloxy)-1H-pyrazole;
(34a) 4-(2-fluoro-4-isopropoxy-benzyl)-1-isopropyl-5-methyl-3-(6-O-methoxycarbonyl-β-D-glucopyranos-1-yloxy)-1H-pyrazole;
(34b) 4-(2-fluoro-4-isopropoxy-benzyl)-1-isopropyl-5-methyl-3-(6-O-ethoxycarbonyl-β-D-glucopyranos-1-yloxy)-1H-pyrazole;
(35a) 4-(2-fluoro-4-ethoxy-benzyl)-1-isopropyl-5-methyl-3-(6-O-methoxycarbonyl-β-D-glucopyranos-1-yloxy)-1H-pyrazole;
(35b) 4-(2-fluoro-4-ethoxy-benzyl)-1-isopropyl-5-methyl-3-(6-O-ethoxycarbonyl-β-D-glucopyranos-1-yloxy)-1H-pyrazole;
(36a) 4-(2-fluoro-4-methoxy-benzyl)-1-isopropyl-5-trifluoromethyl-3-(6-O-methoxycarbonyl-β-D-glucopyranos-1-yloxy)-1H-pyrazole;
(36b) 4-(2-fluoro-4-methoxy-benzyl)-1-isopropyl-5-trifluoromethyl-3-(6-O-ethoxycarbonyl-β-D-glucopyranos-1-yloxy)-1H-pyrazole;
(37a) 4-(2,6-difluoro-4-methoxy-benzyl)-1-isopropyl-5-methyl-3-(6-O-methoxycarbonyl-β-D-glucopyranos-1-yloxy)-1H-pyrazole;
(37b) 4-(2,6-difluoro-4-methoxy-benzyl)-1-isopropyl-5-methyl-3-(6-O-ethoxycarbonyl-β-D-glucopyranos-1-yloxy)-1H-pyrazole;
(38a) 4-(3,5-difluoro-4-methoxy-benzyl)-1-isopropyl-5-methyl-3-(6-O-methoxycarbonyl-β-D-glucopyranos-1-yloxy)-1H-pyrazole;
(38b) 4-(3,5-difluoro-4-methoxy-benzyl)-1-isopropyl-5-methyl-3-(6-O-ethoxycarbonyl-β-D-glucopyranos-1-yloxy)-1H-pyrazole;
(39a) 1-cyclobutyl-4-(3-fluoro-4-methyl-benzyl)-5-methyl-3-(6-O-methoxycarbonyl-β-D-glucopyranos-1-yloxy)-1H-pyrazole;
(39b) 1-cyclobutyl-4-(3-fluoro-4-methyl-benzyl)-5-methyl-3-(6-O-ethoxycarbonyl-β-D-glucopyranos-1-yloxy)-1H-pyrazole;
(40a) 1-cyclopropylmethyl-4-(3-fluoro-4-methyl-benzyl)-5-methyl-3-(6-methoxycarbonyl-β-D-glucopyranos-1-yloxy)-1H-pyrazole;
(40b) 1-cyclopropylmethyl-4-(3-fluoro-4-methyl-benzyl)-5-methyl-3-(6-ethoxycarbonyl-β-D-glucopyranos-1-yloxy)-1H-pyrazole;
(41a) 1-cyclobutyl-4-(2-fluoro-4-methoxy-benzyl)-5-methyl-3-(6-O-methoxycarbonyl-β-D-glucopyranos-1-yloxy)-1H-pyrazole;
(41b) 1-cyclobutyl-4-(2-fluoro-4-methoxy-benzyl)-5-methyl-3-(6-O-ethoxycarbonyl-β-D-glucopyranos-1-yloxy)-1H-pyrazole;
(42a) 4-(4-bromo-3-fluoro-benzyl)-1-isopropyl-5-methyl-3-(6-O-methoxycarbonyl-β-D-glucopyranos-1-yloxy)-1H-pyrazole;
(42b) 4-(4-bromo-3-fluoro-benzyl)-1-isopropyl-5-methyl-3-(6-O-ethoxycarbonyl-β-D-glucopyranos-1-yloxy)-1H-pyrazole;
(43a) 4-(2,3-difluoro-4-methyl-benzyl)-1-isopropyl-5-methyl-3-(6-O-methoxycarbonyl-β-D-glucopyranos-1-yloxy)-1H-pyrazole;
(43b) 4-(2,3-difluoro-4-methyl-benzyl)-1-isopropyl-5-methyl-3-(6-O-ethoxycarbonyl-β-D-glucopyranos-1-yloxy)-1H-pyrazole;
(44a) 4-(4-bromo-2-fluoro-benzyl)-1-isopropyl-5-methyl-3-(6-O-methoxycarbonyl-β-D-glucopyranos-1-yloxy)-1H-pyrazole;
(44b) 4-(4-bromo-2-fluoro-benzyl)-1-isopropyl-5-methyl-3-(6-O-ethoxycarbonyl-β-D-glucopyranos-1-yloxy)-1H-pyrazole;
(45a) 4-(2-fluoro-4-methyl-benzyl)-1-isopropyl-5-methyl-3-(6-O-methoxycarbonyl-β-D-glucopyranos-1-yloxy)-1H-pyrazole;
(46) 4-(3-Fluoro-4-methyl-benzyl)-1-isopropyl-5-methyl-3-(6-O-ethoxycarbonyl-β-D-glucopyranos-1-yloxy)-1H-pyrazole;
(47) 4-(2-Fluoro-4-methoxy-benzyl)-1-isopropyl-5-methyl-3-(6-O-ethoxycarbonyl-β-D-glucopyranos-1-yloxy)-1H-pyrazole;
(48) 4-(2-Fluoro-4-methoxy-benzyl)-1-isopropyl-5-methyl-3-(6-O-isobutyloxycarbonyl-β-D-glucopyranos-1-yloxy)-1H-pyrazole;
(49) 4-(2-Fluoro-4-methoxy-benzyl)-1-isopropyl-5-methyl-3-(6-O-hex-1-yloxycarbonyl-β-D-glucopyranos-1-yloxy)-1H-pyrazole;
(50) 4-(2-Fluoro-4-methoxy-benzyl)-1-isopropyl-5-methyl-3-(6-O-phenoxycarbonyl-β-D-glucopyranos-1-yloxy)-1H-pyrazole;
(51) 4-(2-Fluoro-4-methoxy-benzyl)-1-isopropyl-5-methyl-3-(6-O-benzyloxycarbonyl-β-D-glucopyranos-1-yloxy)-1H-pyrazole;
(52) 4-(2-Fluoro-4-methoxy-benzyl)-1-isopropyl-5-methyl-3-(6-O-acetyl-β-D-glucopyranos-1-yloxy)-1H-pyrazole;
(53) 4-(2-Fluoro-4-methoxy-benzyl)-1-isopropyl-5-methyl-3-(6-O-propylcarbonyl-β-D-glucopyranos-1-yloxy)-1H-pyrazole;
(54) 4-(2-Fluoro-4-methoxy-benzyl)-1-isopropyl-5-methyl-3-(6-O-isopropylcarbonyl-β-D-glucopyranos-1-yloxy)-1H-pyrazole;
(55) 4-(2-Fluoro-4-methoxy-benzyl)-1-isopropyl-5-methyl-3-(6-O-benzylcarbonyl-β-D-glucopyranos-1-yloxy)-1H-pyrazole;
(56) 4-(4-ethyl-benzyl)-1-isopropyl-5-trifluoromethyl-3-(6-O-ethoxycarbonyl-β-D-glucopyranos-1-yloxy)-1H-pyrazole;
(57) 4-(4-bromo-benzyl)-1-isopropyl-5-trifluoromethyl-3-(6-O-ethoxycarbonyl-β-D-glucopyranos-1-yloxy)-1H-pyrazole;
(58) 4-(4-ethyl-benzyl)-1-cyclobutyl-5-trifluoromethyl-3-(6-O-ethoxycarbonyl-β-D-glucopyranos-1-yloxy)-1H-pyrazole;
(59) 4-(4-ethyl-benzyl)-1-(2-fluoro-1-fluoromethyl-ethyl)-5-trifluoromethyl-3-(6-O-ethoxycarbonyl-β-D-glucopyranos-1-yloxy)-1H-pyrazole;
(60) 4-(3-fluoro-4-methoxy-benzyl)-1-isopropyl-5-trifluoromethyl-3-(6-O-ethoxycarbonyl-β-D-glucopyranos-1-yloxy)-1H-pyrazole;
(61) 4-(4-isopropoxy-benzyl)-1-isopropyl-5-methyl-3-(6-O-ethoxycarbonyl-β-D-glucopyranos-1-yloxy)-1H-pyrazole;
(62) 4-(3-fluoro-4-methoxy-benzyl)-1-isopropyl-5-methyl-3-(6-O-ethoxycarbonyl-β-D-glucopyranos-1-yloxy)-1H-pyrazole;
(63) 4-(4-ethyl-benzyl)-1-isopropyl-5-methyl-3-(6-O-ethoxycarbonyl-β-D-glucopyranos-1-yloxy)-1H-pyrazole;
(64) 4-(3-fluoro-4-methoxy-benzyl)-1-isopropyl-5-methyl-3-(6-O-methoxycarbonyl-β-D-glucopyranos-1-yloxy)-1H-pyrazole;
(65) 4-(4-ethyl-benzyl)-1-isopropyl-5-trifluoromethyl-3-(6-O-methoxycarbonyl-β-D-glucopyranos-1-yloxy)-1H-pyrazole;
(66) 4-(4-bromo-benzyl)-1-isopropyl-5-trifluoromethyl-3-(6-O-methoxycarbonyl-β-D-glucopyranos-1-yloxy)-1H-pyrazole;
(67) 4-(4-ethyl-benzyl)-1-cyclobutyl-5-trifluoromethyl-3-(6-O-methoxycarbonyl-β-D-glucopyranos-1-yloxy)-1H-pyrazole;
(68) 4-(4-ethyl-benzyl)-1-(2-fluoro-1-fluoromethyl-ethyl)-5-trifluoromethyl-3-(6-O-methoxycarbonyl-β-D-glucopyranos-1-yloxy)-1H-pyrazole;
(69) 4-(3-fluoro-4-methoxy-benzyl)-1-isopropyl-5-trifluoromethyl-3-(6-O-methoxycarbonyl-β-D-glucopyranos-1-yloxy)-1H-pyrazole;
(70) 4-(3-fluoro-4-methyl-benzyl)-1-isopropyl-5-methyl-3-(6-O-methoxycarbonyl-β-D-glucopyranos-1-yloxy)-1H-pyrazole;
(71) 4-(4-isopropoxy-benzyl)-1-isopropyl-5-methyl-3-(6-O-methoxycarbonyl-β-D-glucopyranos-1-yloxy)-1H-pyrazole;
(72) 4-(2-fluoro-4-methoxy-benzyl)-1-isopropyl-5-methyl-3-(6-O-methoxycarbonyl-β-D-glucopyranos-1-yloxy)-1H-pyrazole;
(73) 4-(4-ethyl-benzyl)-1-isopropyl-5-methyl-3-(6-O-methoxycarbonyl-β-D-glucopyranos-1-yloxy)-1H-pyrazole;
or a pharmaceutically acceptable salt thereof.

16. Use of a pyrazole-O-glucoside derivative selected from the group of compounds (1) to (29),
4-(2,3-difluoro-4-methoxy-benzyl)-1-isopropyl-5-methyl-3-β-D-glucopyranos-1-yloxy-1H-pyrazole;
4-(2,5-difluoro-4-methoxy-benzyl)-1-isopropyl-5-methyl-3-β-D-glucopyranos-1-yloxy-1H-pyrazole;
4-(2,6-difluoro-4-methoxy-benzyl)-1-isopropyl-5-methyl-3-β-D-glucopyranos-1-yloxy-1H-pyrazole;
4-(3,5-difluoro-4-methoxy-benzyl)-1-isopropyl-5-methyl-3-β-D-glucopyranos-1-yloxy-1H-pyrazole;
1-cyclobutyl-4-(3-fluoro-4-methyl-benzyl)-5-methyl-3-β-D-glucopyranos-1-yloxy-1H-pyrazole;
1-cyclopropylmethyl-4-(3-fluoro-4-methyl-benzyl)-5-methyl-3-β-D-glucopyranos-1-yloxy-1H-pyrazole;
1-cyclobutyl-4-(2-fluoro-4-methoxy-benzyl)-5-methyl-3-β-D-glucopyranos-1-yloxy-1H-pyrazole;
4-(3-chloro-4-methoxy-benzyl)-1-isopropyl-5-methyl-3-β-D-glucopyranos-1-yloxy-1H-pyrazole;
4-(2-chloro-4-methoxy-benzyl)-1-isopropyl-5-methyl-3-β-D-glucopyranos-1-yloxy-1H-pyrazole;
4-(4-bromo-3-fluoro-benzyl)-1-isopropyl-5-methyl-3-β-D-glucopyranos-1-yloxy-1H-pyrazole;
4-(2,3-difluoro-4-methyl-benzyl)-1-isopropyl-5-methyl-3-β-D-glucopyranos-1-yloxy-1H-pyrazole;
4-(2-fluoro-4-methyl-benzyl)-1-isopropyl-5-methyl-3-β-D-glucopyranos-1-yloxy-1H-pyrazole;
4-(3-fluoro-4-ethoxy-benzyl)-1-isopropyl-5-methyl-3-β-D-glucopyranos-1-yloxy-1H-pyrazole;
4-(4-ethinyl-benzyl)-1-isopropyl-5-methyl-3-β-D-glucopyranos-1-yloxy-1H-pyrazole;
4-(3-fluoro-4-isopropoxy-benzyl)-1-isopropyl-5-methyl-3-β-D-glucopyranos-1-yloxy-1H-pyrazole;
4-(2-fluoro-4-methoxy-benzyl)-1-isopropyl-5-methyl-3-β-D-glucopyranos-1-yloxy-1H-pyrazole;
4-(2-fluoro-4-methoxy-benzyl)-1-isopropyl-5-trifluoromethyl-3-β-D-glucopyranos-1-yloxy-1H-pyrazole;
4-(4-bromo-2-fluoro-benzyl)-1-isopropyl-5-methyl-3-β-D-glucopyranos-1-yloxy-1H-pyrazole;
4-(2-fluoro-4-isopropoxy-benzyl)-1-isopropyl-5-methyl-3-β-D-glucopyranos-1-yloxy-1H-pyrazole;
4-(2-fluoro-4-ethoxy-benzyl)-1-isopropyl-5-methyl-3-β-D-glucopyranos-1-yloxy-1H-pyrazole;
4-(4-ethyl-benzyl)-1-isopropyl-5-trifluoromethyl-3-β-D-glucopyranos-1-yloxy-1H-pyrazole;
4-(4-bromo-benzyl)-1-isopropyl-5-trifluoromethyl-3-β-D-glucopyranos-1-yloxy-1H-pyrazole;
4-(4-ethyl-benzyl)-1-cyclobutyl-5-trifluoromethyl-3-β-D-glucopyranos-1-yloxy-1H-pyrazole;
4-(4-ethyl-benzyl)-1-(2-fluoro-1-fluoromethyl-ethyl)-5-trifluoromethyl-3-β-D-glucopyranos-1-yloxy-1H-pyrazole;
4-(3-fluoro-4-methoxy-benzyl)-1-isopropyl-5-trifluoromethyl-3-β-D-glucopyranos-1-yloxy-1H-pyrazole;
4-(3-fluoro-4-methyl-benzyl)-1-isopropyl-5-methyl-3-β-D-glucopyranos-1-yloxy-1H-pyrazole;
4-(2,3-difluoro-4-isopropoxy-benzyl)-1-isopropyl-5-methyl-3-β-D-glucopyranos-1-yloxy-1H-pyrazole;
4-(3-fluoro-4-methoxy-benzyl)-1-isopropyl-5-methyl-3-β-D-glucopyranos-1-yloxy-1H-pyrazole;
4-(4-ethyl-benzyl)-1-isopropyl-5-methyl-3-β-D-glucopyranos-1-yloxy-1H-pyrazole;
or a prodrug thereof, or a pharmaceutically acceptable salt thereof, defined as in claim 1 for the manufacture of a medicament for a therapeutic method according to claim 1.

17. Medicament or pharmaceutical composition for use in a method according to claim 1 comprising a therapeutically or prophylactically effective amount of a pyrazole-O-glucoside derivative selected from the group of compounds 1 to 29:
4-(2,3-difluoro-4-methoxy-benzyl)-1-isopropyl-5-methyl-3-β-D-glucopyranos-1-yloxy-1H-pyrazole;
4-(2,5-difluoro-4-methoxy-benzyl)-1-isopropyl-5-methyl-3-β-D-glucopyranos-1-yloxy-1H-pyrazole;
4-(2,6-difluoro-4-methoxy-benzyl)-1-isopropyl-5-methyl-3-β-D-glucopyranos-1-yloxy-1H-pyrazole;
4-(3,5-difluoro-4-methoxy-benzyl)-1-isopropyl-5-methyl-3-β-D-glucopyranos-1-yloxy-1H-pyrazole;
1-cyclobutyl-4-(3-fluoro-4-methyl-benzyl)-5-methyl-3-β-D-glucopyranos-1-yloxy-1H-pyrazole;
1-cyclopropymethyl-4-(3-fluoro-4-methyl-benzyl)-5-methyl-3-β-D-glucopyranos-1-yloxy-1H-pyrazole;
1-cyclobutyl-4-(2-fluoro-4-methoxy-benzyl)-5-methyl-3-β-D-glucopyranos-1-yloxy-1H-pyrazole;
4-(3-chloro-4-methoxy-benzyl)-1-isopropyl-5-methyl-3-β-D-glucopyranos-1-yloxy-1H-pyrazole;
4-(2-chloro-4-methoxy-benzyl)-1-isopropyl-5-methyl-3-β-D-glucopyranos-1-yloxy-1H-pyrazole;
4-(4-bromo-3-fluoro-benzyl)-1-isopropyl-5-methyl-3-β-D-glucopyranos-1-yloxy-1H-pyrazole;
4-(2,3-difluoro-4-methyl-benzyl)-1-isopropyl-5-methyl-3-β-D-glucopyranos-1-yloxy-1H-pyrazole;
4-(2-fluoro-4-methyl-benzyl)-1-isopropyl-5-methyl-3-β-D-glucopyranos-1-yloxy-1H-pyrazole;
4-(3-fluoro-4-ethoxy-benzyl)-1-isopropyl-5-methyl-3-β-D-glucopyranos-1-yloxy-1H-pyrazole;
4-(4-ethinyl-benzyl)-1-isopropyl-5-methyl-3-β-D-glucopyranos-1-yloxy-1H-pyrazole;
4-(3-fluoro-4-isopropoxy-benzyl)-1-isopropyl-5-methyl-3-β-D-glucopyranos-1-yloxy-1H-pyrazole;
4-(2-fluoro-4-methoxy-benzyl)-1-isopropyl-5-methyl-3-β-D-glucopyranos-1-yloxy-1H-pyrazole;
4-(2-fluoro-4-methoxy-benzyl)-1-isopropyl-5-trifluoromethyl-3-β-D-glucopyranos-1-yloxy-1H-pyrazole;
4-(4-bromo-2-fluoro-benzyl)-1-isopropyl-5-methyl-3-β-D-glucopyranos-1-yloxy-1H-pyrazole;
4-(2-fluoro-4-isopropoxy-benzyl)-1-isopropyl-5-methyl-3-β-D-glucopyranos-1-yloxy-1H-pyrazole;
4-(2-fluoro-4-ethoxy-benzyl)-1-isopropyl-5-methyl-3-β-D-glucopyranos-1-yloxy-1H-pyrazole;
4-(4-ethyl-benzyl)-1-isopropyl-5-trifluoromethyl-3-β-D-glucopyranos-1-yloxy-1H-pyrazole;
4-(4-bromo-benzyl)-1-isopropyl-5-trifluoromethyl-3-β-D-glucopyranos-1-yloxy-1H-pyrazole;
4-(4-ethyl-benzyl)-1-cyclobutyl-5-trifluoromethyl-3-β-D-glucopyranos-1-yloxy-1H-pyrazole;
4-(4-ethyl-benzyl)-1-(2-fluoro-1-fluoromethyl-ethyl)-5-trifluoromethyl-3-β-D-glucopyranos-1-yloxy-1H-pyrazole;
4-(3-fluoro-4-methoxy-benzyl)-1-isopropyl-5-trifluoromethyl-3-β-D-glucopyranos-1-yloxy-1H-pyrazole;
4-(3-fluoro-4-methyl-benzyl)-1-isopropyl-5-methyl-3-β-D-glucopyranos-1-yloxy-1H-pyrazole;
4-(2,3-difluoro-4-isopropoxy-benzyl)-1-isopropyl-5-methyl-3-β-D-glucopyranos-1-yloxy-1H-pyrazole;
4-(3-fluoro-4-methoxy-benzyl)-1-isopropyl-5-methyl-3-β-D-glucopyranos-1-yloxy-1H-pyrazole;
4-(4-ethyl-benzyl)-1-isopropyl-5-methyl-3-β-D-glucopyranos-1-yloxy-1H-pyrazole;
or a prodrug thereof, or a pharmaceutically acceptable salt thereof, defined as in claim 1.

18. Pyrazole-O-glucoside derivative selected from the group consisting of:
(1) 4-(2,3-difluoro-4-methoxy-benzyl)-1-isopropyl-5-methyl-3-β-D-glucopyranos-1-yloxy-1H-pyrazole;
(2) 4-(2,5-difluoro-4-methoxy-benzyl)-1-isopropyl-5-methyl-3-β-D-glucopyranos-1-yloxy-1H-pyrazole;
(3) 4-(2,6-difluoro-4-methoxy-benzyl)-1-isopropyl-5-methyl-3-β-D-glucopyranos-1-yloxy-1H-pyrazole;
(4) 4-(3,5-difluoro-4-methoxy-benzyl)-1-isopropyl-5-methyl-3-β-D-glucopyranos-1-yloxy-1H-pyrazole;
(5) 1-cyclobutyl-4-(3-fluoro-4-methyl-benzyl)-5-methyl-3-β-D-glucopyranos-1-yloxy-1H-pyrazole;
(6) 1-cyclopropylmethyl-4-(3-fluoro-4-methyl-benzyl)-5-methyl-3-β-D-glucopyranos-1-yloxy-1H-pyrazole;
(7) 1-cyclobutyl-4-(2-fluoro-4-methoxy-benzyl)-5-methyl-3-β-D-glucopyranos-1-yloxy-1H-pyrazole;
(8) 4-(3-chloro-4-methoxy-benzyl)-1-isopropyl-5-methyl-3-β-D-glucopyranos-1-yloxy-1H-pyrazole;
(9) 4-(2-chloro-4-methoxy-benzyl)-1-isopropyl-5-methyl-3-β-D-glucopyranos-1-yloxy-1H-pyrazole;
(10) 4-(4-bromo-3-fluoro-benzyl)-1-isopropyl-5-methyl-3-β-D-glucopyranos-1-yloxy-1H-pyrazole;
(11) 4-(2,3-difluoro-4-methyl-benzyl)-1-isopropyl-5-methyl-3-β-D-glucopyranos-1-yloxy-1H-pyrazole;
(12) 4-(2-fluoro-4-methyl-benzyl)-1-isopropyl-5-methyl-3-β-D-glucopyranos-1-yloxy-1H-pyrazole;
(13) 4-(3-fluoro-4-ethoxy-benzyl)-1-isopropyl-5-methyl-3-β-D-glucopyranos-1-yloxy-1H-pyrazole;
(14) 4-(4-ethinyl-benzyl)-1-isopropyl-5-methyl-3-β-D-glucopyranos-1-yloxy-1H-pyrazole;
(15) 4-(3-fluoro-4-isopropoxy-benzyl)-1-isopropyl-5-methyl-3-β-D-glucopyranos-1-yloxy-1H-pyrazole;
(17) 4-(2-fluoro-4-methoxy-benzyl)-1-isopropyl-5-trifluoromethyl-3-β-D-glucopyranos-1-yloxy-1H-pyrazole;
(18) 4-(4-bromo-2-fluoro-benzyl)-1-isopropyl-5-methyl-3-β-D-glucopyranos-1-yloxy-1H-pyrazole;
(19) 4-(2-fluoro-4-isopropoxy-benzyl)-1-isopropyl-5-methyl-3-β-D-glucopyranos-1-yloxy-1H-pyrazole;
(20) 4-(2-fluoro-4-ethoxy-benzyl)-1-isopropyl-5-methyl-3-β-D-glucopyranos-1-yloxy-1H-pyrazole;
or a prodrug thereof wherein one or more hydroxyl groups of the β-D-glucopyranosyl group are acylated with groups selected from (C₁₋₁₈-alkyl)carbonyl, (C₁₋₁₈-alkyl)oxycarbonyl, phenylcarbonyl, phenyl-(C₁₋₃-alkyl)-carbonyl, phenyloxycarbonyl and phenyl-(C₁₋₃-alkyl)-oxycarbonyl, or a pharmaceutically acceptable salt thereof.

19. Pyrazole-O-glucoside derivative according to claim 18 characterized in that it is a prodrug, wherein the hydrogen atom of the hydroxyl group in 6-position of the β-D-glucopyranosyl-group is replaced by a group selected from among (C₁₋₃-alkyl)carbonyl, (C₁₋₆-alkyl)oxycarbonyl, phenyloxycarbonyl, benzyloxycarbonyl and benzylcarbonyl, or a pharmaceutically acceptable salt thereof.

20. Pyrazole-O-glucoside derivative according to claim 19 selected from the group consisting of
(30a) 4-(2,3-difluoro-4-methoxy-benzyl)-1-isopropyl-5-methyl-3-(6-O-methoxycarbonyl-β-D-glucopyranos-1-yloxy)-1H-pyrazole;
(30b) 4-(2,3-difluoro-4-methoxy-benzyl)-1-isopropyl-5-methyl-3-(6-O-ethoxycarbonyl-β-D-glucopyranos-1-yloxy)-1H-pyrazole;
(31a) 4-(3-fluoro-4-ethoxy-benzyl)-1-isopropyl-5-methyl-3-(6-O-methoxycarbonyl-β-D-glucopyranos-1-yloxy)-1H-pyrazole;
(31b) 4-(3-fluoro-4-ethoxy-benzyl)-1-isopropyl-5-methyl-3-(6-O-ethoxycarbonyl-β-D-glucopyranos-1-yloxy)-1H-pyrazole;
(32a) 4-(3-fluoro-4-isopropoxy-benzyl)-1-isopropyl-5-methyl-3-(6-O-methoxycarbonyl-β-D-glucopyranos-1-yloxy)-1H-pyrazole;
(32b) 4-(3-fluoro-4-isopropoxy-benzyl)-1-isopropyl-5-methyl-3-(6-O-ethoxycarbonyl-β-D-glucopyranos-1-yloxy)-1H-pyrazole;
(33a) 4-(2,5-difluoro-4-methoxy-benzyl)-1-isopropyl-5-methyl-3-(6-O-methoxycarbonyl-β-D-glucopyranos-1-yloxy)-1H-pyrazole;
(33b) 4-(2,5-difluoro-4-methoxy-benzyl)-1-isopropyl-5-methyl-3-(6-O-ethoxycarbonyl-β-D-glucopyranos-1-yloxy)-1H-pyrazole;
(34a) 4-(2-fluoro-4-isopropoxy-benzyl)-1-isopropyl-5-methyl-3-(6-O-methoxycarbonyl-β-D-glucopyranos-1-yloxy)-1H-pyrazole;
(34b) 4-(2-fluoro-4-isopropoxy-benzyl)-1-isopropyl-5-methyl-3-(6-O-ethoxycarbonyl-β-D-glucopyranos-1-yloxy)-1H-pyrazole;
(35a) 4-(2-fluoro-4-ethoxy-benzyl)-1-isopropyl-5-methyl-3-(6-O-methoxycarbonyl-β-D-glucopyranos-1-yloxy)-1H-pyrazole;
(35b) 4-(2-fluoro-4-ethoxy-benzyl)-1-isopropyl-5-methyl-3-(6-O-ethoxycarbonyl-β-D-glucopyranos-1-yloxy)-1H-pyrazole;
(36a) 4-(2-fluoro-4-methoxy-benzyl)-1-isopropyl-5-trifluoromethyl-3-(6-O-methoxycarbonyl-β-D-glucopyranos-1-yloxy)-1H-pyrazole;
(36b) 4-(2-fluoro-4-methoxy-benzyl)-1-isopropyl-5-trifluoromethyl-3-(6-O-ethoxycarbonyl-β-D-glucopyranos-1-yloxy)-1H-pyrazole;
(37a) 4-(2,6-difluoro-4-methoxy-benzyl)-1-isopropyl-5-methyl-3-(6-O-methoxycarbonyl-β-D-glucopyranos-1-yloxy)-1H-pyrazole;
(37b) 4-(2,6-difluoro-4-methoxy-benzyl)-1-isopropyl-5-methyl-3-(6-O-ethoxycarbonyl-β-D-glucopyranos-1-yloxy)-1H-pyrazole;
(38a) 4-(3,5-difluoro-4-methoxy-benzyl)-1-isopropyl-5-methyl-3-(6-O-methoxycarbonyl-β-D-glucopyranos-1-yloxy)-1H-pyrazole;
(38b) 4-(3,5-difluoro-4-methoxy-benzyl)-1-isopropyl-5-methyl-3-(6-O-ethoxycarbonyl-β-D-glucopyranos-1-yloxy)-1H-pyrazole;
(39a) 1-cyclobutyl-4-(3-fluoro-4-methyl-benzyl)-5-methyl-3-(6-O-methoxycarbonyl-β-D-glucopyranos-1-yloxy)-1H-pyrazole;
(39b) 1-cyclobutyl-4-(3-fluoro-4-methyl-benzyl)-5-methyl-3-(6-O-ethoxycarbonyl-β-D-glucopyranos-1-yloxy)-1H-pyrazole;
(40a) 1-cyclopropylmethyl-4-(3-fluoro-4-methyl-benzyl)-5-methyl-3-(6-methoxycarbonyl-β-D-glucopyranos-1-yloxy)-1H-pyrazole;
(40b) 1-cyclopropylmethyl-4-(3-fluoro-4-methyl-benzyl)-5-methyl-3-(6-ethoxycarbonyl-β-D-glucopyranos-1-yloxy)-1H-pyrazole;
(41a) 1-cyclobutyl-4-(2-fluoro-4-methoxy-benzyl)-5-methyl-3-(6-O-methoxycarbonyl-β-D-glucopyranos-1-yloxy)-1H-pyrazole;
(41b) 1-cyclobutyl-4-(2-fluoro-4-methoxy-benzyl)-5-methyl-3-(6-O-ethoxycarbonyl-β-D-glucopyranos-1-yloxy)-1H-pyrazole;
(42a) 4-(4-bromo-3-fluoro-benzyl)-1-isopropyl-5-methyl-3-(6-O-methoxycarbonyl-β-D-glucopyranos-1-yloxy)-1H-pyrazole;
(42b) 4-(4-bromo-3-fluoro-benzyl)-1-isopropyl-5-methyl-3-(6-O-ethoxycarbonyl-β-D-glucopyranos-1-yloxy)-1H-pyrazole;
(43a) 4-(2,3-difluoro-4-methyl-benzyl)-1-isopropyl-5-methyl-3-(6-O-methoxycarbonyl-β-D-glucopyranos-1-yloxy)-1H-pyrazole;
(43b) 4-(2,3-difluoro-4-methyl-benzyl)-1-isopropyl-5-methyl-3-(6-O-ethoxycarbonyl-β-D-glucopyranos-1-yloxy)-1H-pyrazole;
(44a) 4-(4-bromo-2-fluoro-benzyl)-1-isopropyl-5-methyl-3-(6-O-methoxycarbonyl-β-D-glucopyranos-1-yloxy)-1H-pyrazole;
(44b) 4-(4-bromo-2-fluoro-benzyl)-1-isopropyl-5-methyl-3-(6-O-ethoxycarbonyl-β-D-glucopyranos-1-yloxy)-1H-pyrazole;
(45a) 4-(2-fluoro-4-methyl-benzyl)-1-isopropyl-5-methyl-3-(6-O-methoxycarbonyl-β-D-glucopyranos-1-yloxy)-1H-pyrazole;
(45b) 4-(2-fluoro-4-methyl-benzyl)-1-isopropyl-5-methyl-3-(6-O-ethoxycarbonyl-β-D-glucopyranos-1-yloxy)-1H-pyrazole;
or pharmaceutically acceptable salts thereof.

21. Pyrazole-O-glucoside derivative selected from the group consisting of:
(46) 4-(3-Fluoro-4-methyl-benzyl)-1-isopropyl-5-methyl-3-(6-O-ethoxycarbonyl-β-D-glucopyranos-1-yloxy)-1H-pyrazole;
(47) 4-(2-Fluoro-4-methoxy-benzyl)-1-isopropyl-5-methyl-3-(6-O-ethoxycarbonyl-β-D-glucopyranos-1-yloxy)-1H-pyrazole;
(48) 4-(2-Fluoro-4-methoxy-benzyl)-1-isopropyl-5-methyl-3-(6-O-isobutyloxycarbonyl-β-D-glucopyranos-1-yloxy)-1H-pyrazole;
(49) 4-(2-Fluoro-4-methoxy-benzyl)-1-isopropyl-5-methyl-3-(6-O-hex-1-yloxycarbonyl-β-D-glucopyranos-1-yloxy)-1H-pyrazole;
(50) 4-(2-Fluoro-4-methoxy-benzyl)-1-isopropyl-5-methyl-3-(6-O-phenoxycarbonyl-β-D-glucopyranos-1-yloxy)-1H-pyrazole;
(51) 4-(2-Fluoro-4-methoxy-benzyl)-1-isopropyl-5-methyl-3-(6-O-benzyloxycarbonyl-β-D-glucopyranos-1-yloxy)-1H-pyrazole;
(52) 4-(2-Fluoro-4-methoxy-benzyl)-1-isopropyl-5-methyl-3-(6-O-acetyl-β-D-glucopyranos-1-yloxy)-1H-pyrazole;
(53) 4-(2-Fluoro-4-methoxy-benzyl)-1-isopropyl-5-methyl-3-(6-O-propylcarbonyl-β-D-glucopyranos-1-yloxy)-1H-pyrazole;
(54) 4-(2-Fluoro-4-methoxy-benzyl)-1-isopropyl-5-methyl-3-(6-O-isopropylcarbonyl-β-D-glucopyranos-1-yloxy)-1H-pyrazole;
(55) 4-(2-Fluoro-4-methoxy-benzyl)-1-isopropyl-5-methyl-3-(6-O-benzylcarbonyl-β-D-glucopyranos-1-yloxy)-1H-pyrazole;
(56) 4-(4-ethyl-benzyl)-1-isopropyl-5-trifluoromethyl-3-(6-O-ethoxycarbonyl-β-D-glucopyranos-1-yloxy)-1H-pyrazole;
(57) 4-(4-bromo-benzyl)-1-isopropyl-5-trifluoromethyl-3-(6-O-ethoxycarbonyl-β-D-glucopyranos-1-yloxy)-1H-pyrazole;
(58) 4-(4-ethyl-benzyl)-1-cyclobutyl-5-trifluoromethyl-3-(6-O-ethoxycarbonyl-β-D-glucopyranos-1-yloxy)-1H-pyrazole;
(59) 4-(4-ethyl-benzyl)-1-(2-fluoro-1-fluoromethyl-ethyl)-5-trifluoromethyl-3-(6-O-ethoxycarbonyl-β-D-glucopyranos-1-yloxy)-1H-pyrazole;
(60) 4-(3-fluoro-4-methoxy-benzyl)-1-isopropyl-5-trifluoromethyl-3-(6-O-ethoxycarbonyl-β-D-glucopyranos-1-yloxy)-1H-pyrazole;
(61) 4-(4-isopropoxy-benzyl)-1-isopropyl-5-methyl-3-(6-O-ethoxycarbonyl-β-D-glucopyranos-1-yloxy)-1H-pyrazole;
(62) 4-(3-fluoro-4-methoxy-benzyl)-1-isopropyl-5-methyl-3-(6-O-ethoxycarbonyl-β-D-glucopyranos-1-yloxy)-1H-pyrazole;
(63) 4-(4-ethyl-benzyl)-1-isopropyl-5-methyl-3-(6-O-ethoxycarbonyl-β-D-glucopyranos-1-yloxy)-1H-pyrazole;
or pharmaceutically acceptable salts thereof.

22. A pharmaceutical composition comprising at least one pyrazole-O-glucoside derivative of claim 18, 19, 20 or 21, or a pharmaceutically acceptable salt thereof.
